SEQUENCE LISTING (61 sequences)

SEQ ID NO: 1 - protein, 15 aa, Artificial Sequence
  Description of Artificial Sequence: Synthetic peptide
RASKGVSTSGYSYLH

SEQ ID NO: 2 - protein, 7 aa, Artificial Sequence
  Description of Artificial Sequence: Synthetic peptide
LASYLES

SEQ ID NO: 3 - protein, 9 aa, Artificial Sequence
  Description of Artificial Sequence: Synthetic peptide
QHSRDLPLT

SEQ ID NO: 4 - protein, 5 aa, Artificial Sequence
  Description of Artificial Sequence: Synthetic peptide
NYYMY

SEQ ID NO: 5 - protein, 17 aa, Artificial Sequence
  Description of Artificial Sequence: Synthetic peptide
GINPSNGGTNFNEKFKN

SEQ ID NO: 6 - protein, 11 aa, Artificial Sequence
  Description of Artificial Sequence: Synthetic peptide
RDYRFDMGFDY

SEQ ID NO: 7 - protein, 111 aa, Artificial Sequence [sequence omitted]

SEQ ID NO: 8 - protein, 120 aa, Artificial Sequence [sequence omitted]

SEQ ID NO: 9 - protein, 218 aa, Artificial Sequence
  Description of Artificial Sequence: Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 447 aa, Artificial Sequence
  Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 12 aa, Artificial Sequence
  Description of Artificial Sequence: Synthetic peptide
RASQSVSSYLAW

SEQ ID NO: 12 - protein, 7 aa, Artificial Sequence
  Description of Artificial Sequence: Synthetic peptide
DASNRAT

SEQ ID NO: 13 - protein, 9 aa, Artificial Sequence
  Description of Artificial Sequence: Synthetic peptide
QQSSNWPRT

SEQ ID NO: 14 - protein, 6 aa, Artificial Sequence
  Description of Artificial Sequence: Synthetic peptide
SNSGMH

SEQ ID NO: 15 - protein, 17 aa, Artificial Sequence
  Description of Artificial Sequence: Synthetic peptide
VIWYDGSKRYYADSVKG

SEQ ID NO: 16 - protein, 4 aa, Artificial Sequence
  Description of Artificial Sequence: Synthetic peptide
NDDY

SEQ ID NO: 17 - protein, 107 aa, Artificial Sequence [sequence omitted]

SEQ ID NO: 18 - protein, 113 aa, Artificial Sequence [sequence omitted]

SEQ ID NO: 19 - protein, 214 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 440 aa, Artificial Sequence
  Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 11 aa, Artificial Sequence
  Description of Artificial Sequence: Synthetic peptide
RASLSINTFLN

SEQ ID NO: 22 - protein, 7 aa, Artificial Sequence
  Description of Artificial Sequence: Synthetic peptide
AASSLHG

SEQ ID NO: 23 - protein, 9 aa, Artificial Sequence
  Description of Artificial Sequence: Synthetic peptide
QQSSNTPFT

SEQ ID NO: 24 - protein, 5 aa, Artificial Sequence
  Description of Artificial Sequence: Synthetic peptide
NFGMT

SEQ ID NO: 25 - protein, 17 aa, Artificial Sequence
  Description of Artificial Sequence: Synthetic peptide
GISGGGRDTYFADSVKG

SEQ ID NO: 26 - protein, 8 aa, Artificial Sequence
  Description of Artificial Sequence: Synthetic peptide
WGNIYFDY

SEQ ID NO: 27 - protein, 107 aa, Artificial Sequence [sequence omitted]

SEQ ID NO: 28 - protein, 117 aa, Artificial Sequence [sequence omitted]

SEQ ID NO: 29 - protein, 214 aa, Artificial Sequence
  Description of Artificial Sequence: Synthetic polypeptide
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 444 aa, Artificial Sequence
  Description of Artificial Sequence: Synthetic polypeptide
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 11 aa, Artificial Sequence
  Description of Artificial Sequence: Synthetic peptide
RASQDVSTAVA

SEQ ID NO: 32 - protein, 7 aa, Artificial Sequence
  Description of Artificial Sequence: Synthetic peptide
SASFLYS

SEQ ID NO: 33 - protein, 9 aa, Artificial Sequence
  Description of Artificial Sequence: Synthetic peptide
QQYLYHPAT

SEQ ID NO: 34 - protein, 5 aa, Artificial Sequence
  Description of Artificial Sequence: Synthetic peptide
DSWIH

SEQ ID NO: 35 - protein, 17 aa, Artificial Sequence
  Description of Artificial Sequence: Synthetic peptide
WISPYGGSTYYADSVKG

SEQ ID NO: 36 - protein, 9 aa, Artificial Sequence
  Description of Artificial Sequence: Synthetic peptide
RHWPGGFDY

SEQ ID NO: 37 - protein, 107 aa, Artificial Sequence [sequence omitted]

SEQ ID NO: 38 - protein, 118 aa, Artificial Sequence [sequence omitted]

SEQ ID NO: 39 - protein, 214 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 448 aa, Artificial Sequence
  Description of Artificial Sequence: Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 14 aa, Artificial Sequence
  Description of Artificial Sequence: Synthetic peptide
TGTSSDVGGYNYVS

SEQ ID NO: 42 - protein, 7 aa, Artificial Sequence
  Description of Artificial Sequence: Synthetic peptide
DVSNRPS

SEQ ID NO: 43 - protein, 10 aa, Artificial Sequence
  Description of Artificial Sequence: Synthetic peptide
SSYTSSSTRV

SEQ ID NO: 44 - protein, 5 aa, Artificial Sequence
  Description of Artificial Sequence: Synthetic peptide
SYIMM

SEQ ID NO: 45 - protein, 17 aa, Artificial Sequence
  Description of Artificial Sequence: Synthetic peptide
SIYPSGGITFYADTVKG

SEQ ID NO: 46 - protein, 11 aa, Artificial Sequence
  Description of Artificial Sequence: Synthetic peptide
IKLGTVTTVDY

SEQ ID NO: 47 - protein, 110 aa, Artificial Sequence
  Description of Artificial Sequence: Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 48 - protein, 120 aa, Artificial Sequence
  Description of Artificial Sequence: Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 49 - protein, 216 aa, Artificial Sequence
  Description of Artificial Sequence: Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 50 - protein, 450 aa, Artificial Sequence
  Description of Artificial Sequence: Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 12 aa, Artificial Sequence
  Description of Artificial Sequence: Synthetic peptide
RASQRVSSSYLA

SEQ ID NO: 52 - protein, 7 aa, Artificial Sequence
  Description of Artificial Sequence: Synthetic peptide
DASSRAT

SEQ ID NO: 53 - protein, 9 aa, Artificial Sequence
  Description of Artificial Sequence: Synthetic peptide
QQYGSLPWT

SEQ ID NO: 54 - protein, 5 aa, Artificial Sequence
  Description of Artificial Sequence: Synthetic peptide
RYWMS

SEQ ID NO: 55 - protein, 17 aa, Artificial Sequence
  Description of Artificial Sequence: Synthetic peptide
NIKQDGSEKYYVDSVKG

SEQ ID NO: 56 - protein, 12 aa, Artificial Sequence
  Description of Artificial Sequence: Synthetic peptide
EGGWFGELAFDY

SEQ ID NO: 57 - protein, 108 aa, Artificial Sequence
  Description of Artificial Sequence: Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 58 - protein, 121 aa, Artificial Sequence
  Description of Artificial Sequence: Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 59 - protein, 215 aa, Artificial Sequence
  Description of Artificial Sequence: Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 451 aa, Artificial Sequence
  Description of Artificial Sequence: Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 20 aa, Artificial Sequence
  Description of Artificial Sequence: Synthetic peptide
  SITE (1)..(10): This region may encompass 1-10 residues
  SITE (11)..(20): This region may encompass 1-10 residues
  See specification as filed for detailed description of substitutions and preferred embodiments
GGGGGGGGGGSSSSSSSSSS

(19) United States
(12) Patent Application Publication
(10) Pub. No.: US 2023/0201161 A1
(43) Pub. Date: Jun. 29, 2023

(54) Combination comprising HDAC inhibitor, LAG-3 inhibitor and a PD-1 inhibitor or PD-L1 inhibitor for cancer treatment

(71) Applicant: 4SC AG, Planegg-Martinsried (DE)

(72) Inventors: René BARTZ, Penzberg (DE); Frank HERMANN, Munich (DE)

(73) Assignee: 4SC AG, Planegg-Martinsried (DE)

(21) Appl. No.: 17/048,447

(22) PCT Filed: Apr. 17, 2019
(86) PCT No.: PCT/EP2019/059940

§ 371 Date: Oct. 16, 2020

(30) Foreign Application Priority Data
Apr. 17, 2018 (EP) 18167719.6

(51) Int. Cl. A61K 31/40; A61P 35/00

(52) U.S. Cl. CPC A61K 31/40; A61P 35/00

(57) ABSTRACT

The invention relates to medical uses of an HDAC inhibitor of the below general formula I, wherein R1 to R7 are as described herein, or a salt or solvate thereof in combination with LAG-3 inhibitor and a PD-1 inhibitor or PD-L1 inhibitor for the treatment of cancer.

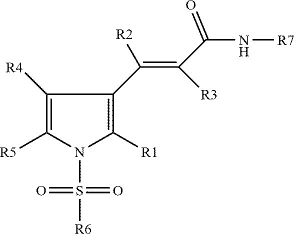

### FIELD OF APPLICATION OF THE INVENTION

The invention relates to medical applications of an HDAC inhibitor in combination with a LAG-3 inhibitor and a PD-1 inhibitor or PD-L1 inhibitor in the treatment of cancer.

### KNOWN TECHNICAL BACKGROUND

The role of the immune system in cancer has gained increasing interest and treatment options harnessing the body’s own immune system have either already received marketing approval or are currently being developed. Of particular interest are treatments that abrogate the tumors’ ability to effectively suppress immune responses by activating negative regulatory pathways (also called checkpoints) that are associated with immune homeostasis or allow the cancer to avoid destruction by the immune system over all. Two such checkpoints, cytotoxic T-lymphocyte protein 4 (CTLA4) and programmed cell death protein 1 (PD-1) and its ligand (PD-L1), have garnered the most attention so far. CTLA4 is a negative regulator of T cells that acts to control T-cell activation by competing with the co-stimulatory molecule CD28 for binding to shared ligands CD80 (also known as B7.1) and CD86 (also known as B7.2). The cell-surface receptor PD-1 is expressed by T cells on activation during priming or expansion and binds to one of two ligands, PD-L1 and PD-L2. Many types of cells can express PD-L1, including tumor cells and immune cells. Binding of PD-L1 or PD-L2 to PD-1 generates an inhibitory signal that attenuates the activity of T cells. Monoclonal antibodies that target either CTLA-4, PD-1 or PD-L1 can block this binding and boost the immune response against cancer cells.

In the field of immuno-oncology, the FDA granted approval for Ipilimumab “for the treatment of unresectable or metastatic melanoma” on 25 Mar. 2011. The EMA granted approval “for the treatment of advanced (unresectable or metastatic) melanoma in adults who have received prior therapy” on 13 Jul. 2011, and subsequently broadened the indication on 31 Oct. 2013 by granting approval for the first-line advanced melanoma. In 2015, the EMA approved with Nivolumab and Pembrolizumab two anti-PD1 antibodies for the treatment of advanced malignant melanoma. The approval for Pembrolizumab was mainly based on the results of the randomized Phase II study (Keynote-002) and a randomized Phase III (Keynote-006) study. The Keynote-002 study was an open-label, multi-center randomized Phase 2 trial to compare two dose levels of Pembrolizumab versus investigator-choice chemotherapy in patients with advanced melanoma. Patients were randomized to receive either Pembrolizumab 2 mg/kg every 3 weeks, Pembrolizumab 10 mg/kg every 3 weeks or investigator-choice chemotherapy.

LAG-3, also designated CD233, is an immune checkpoint receptor on T cells LAG-3’s main ligand is MHC (major histocompatibility complex) II, to which it binds with higher affinity than CD4 (Huard et al, 1995 European Journal of Immunology, 25, 2718-2721). The binding of MHC class II to LAG-3 results in reduced cellular proliferation, activation and homeostasis of T cells, LAG-3 also help to maintain CD8+ T cells in a tolerogenic state, and working with PD-1, helps maintain CD8 exhaustion during chronic viral infection (Workman & Vignali, 2003 European Journal of Immunology, 33, 970-979; Workman et al, 2004 The Journal of Immunology, 172, 5450-5455; Blackburn et al, 2009 Nature Immunology, 10, 29-37).

Histone deacetylases (HDACs) are enzymes that catalyze the removal of acetyl groups from specific histone sites in particular at promotor and enhancer regions, which is an essential part of regulation of cellular gene transcription. HDACs also regulate gene expression in an indirect fashion by mediating the acetylation of non-histone proteins such as DNA-binding proteins, transcription factors, signal transducers, DNA repair and chaperon proteins (Ververis K et al., Biologics: Targets and Therapy 7: 47-60, 2013; Vitt D et al., Targeting histone acetylation. In: RSC Drug Discovery Series No. 48: Epigenetics for Drug Discovery. Editor: Nessa Carey. The Royal Society of Chemistry, 2016).

HDAC inhibitors have been described to cause growth arrest with subsequent differentiation or apoptosis of tumor cells, whereas normal cells are not affected. As summarized in a review article by Marks et al. (Nature Reviews Cancer, 2001, Volume 1, page 194-202), HDAC inhibitors cause cell-cycle arrest in G1 and/or G2 phase. Growth-inhibitory effects have been documented in vitro in virtually all transformed cell types, including cell lines that arise from both hematological and epithelial tumors. The growth inhibitory cellular mechanism of the HDAC inhibitors has been described as a specific induction of expression of the cell cycle inhibitor CDKN1A (p21). Additionally, this review article summarizes the induction of growth arrest in tumor-bearing mice by HDAC inhibitors. Efficacy of HDAC inhibitors has been demonstrated in animal models of diverse cancer types such as breast, prostate, lung and stomach cancers, neuroblastoma and leukemia.

Treatments of many cancer types by HDAC inhibitors have been described in the available literature. HDAC inhibition has an effect on the expression of a number of proteins playing pivotal roles in tumor-relevant processes, such as HER2/neu, VEGF, raf-1, cyclin A and B, Bax, Bad, p53, c-myc, Caspase 3, p21 and ERα. According to a review by Villar-Garea et al. (Int. J. Cancer: 112, 171-178 (2004)) cancer is understood to be an epigenetic as well as a genetic disease and the main goal using HDAC inhibitors would be restoration of gene expression of those tumor-suppressor genes that have been transcriptionally silenced by promotor-associated histone deacetylation. Drummond et al. (Annu. Rev. Pharmacol. Toxicol. 2005. 45:495-528) review the molecular mechanism and outcome of histone and non-histone substrates in cancer cells, which are effectors of HDAC, while HDAC also facilitates the acetylation of several key proteins other than histones. According to said review, acetylation is a key posttranslational modification of many proteins responsible for regulating critical intracellular pathways, and many of these substrates are tissue/development specific (EKLF, GATA-1, ERα, MyoD), oncogenic (c-Myb), tumor-suppressing (p53), or even rather ubiquitous (TFIIE, TFIIF, TCF, HNF-4) transcription factors. Modulation of those proteins can lead to induction of cell cycle arrest, differentiation and apoptosis, all of which are desirable mechanisms for treatment of cancer. Kelly et al. (Expert Opin Invest Drugs, 11(12), 2002) provides a further review on HDAC inhibitors in general and their application in cancer therapy.

The official US NIH website http://clinicaltrials.gov lists (status: February 2016) 545 clinical trials for cancer indications treated with HDAC inhibitors, among others various forms of leukemia (e.g. CML, CLL, AML), myelodysplastic syndrome, lymphoma including non-hodgkin’s lymphoma, multiple myeloma, plasma cell neoplasm, solid tumors in general, small intestine cancer, mesothelioma, prostate, breast (male and female), lung cancer (including non-small and small cell), neuroendocrine, malignant epithelial neoplasms, pancreas, skin cancer (including melanoma), multiple myeloma, cervix, renal cell, head and neck, gastric, ovarian, liver cancer, colon, rectal, thymoma, fallopian tube, peritoneal, nasopharyngeal, vestibular schwannoma, meningioma, acoustic neuroma, neurofibromatosis type 2, thyroid, urothelial, gliomas, brain, esophagus, astrocytoma, anaplastic oligodendroglioma, giant cell glioblastoma, glioblastoma, gliosarcoma, mixed glioma and brain neoplasm.

4SC-202 (E)-N-(2-aminophenyl)-3-(1-((4-(1-methyl-1H-pyrazol-4-yl)phenyl)sulfonyl)-1H-pyrrol-3-yl)acrylamide is an orally available HDAC inhibitor histone-deacetylase (HDAC) inhibitor.

4SC-202 has been evaluated in a Phase I clinical trial (TOPAS) in 24 heavily pre-treated patients with different types of blood cancer. 4SC-202 was well tolerated with few and/or manageable adverse events. Positive signs of anti-tumor efficacy were observed with one complete remission for 28 months and one partial responder for 8 months. Findings also exhibited disease control in 83% of the patients and long-term stabilization in 50% of patients.

WO 2006/097474 A1 describes certain N-sulphonylpyrrole derivatives and their medical utility. WO 2009/112522 A1 describes salts of certain N-sulphonylpyrrole derivatives and their medical utility.

### BRIEF DESCRIPTION OF THE FIGURES

FIG. 1: C38 model as per example section, mean values over all animals of each group per time point, days on the x-axis: days, tumor volume (mm³) on the y-axis. Standard deviations are removed for better legibility; for an indication of standard deviations, see FIG. 2 with box plots for day 27 data point. A: vehicle, B. anti-LAG-3, C. 4SC-202, D. anti-PD1 + anti-LAG3, E. anti-PD-1, F. 4SC-202 + anti-LAG3, G. 4SC-202 + anti-PD-1, H. 4SC-202 + anti-PD-1 + anti-LAG3.

FIG. 2: Box plot for day 27 data point of C38 model as per example section, mean values over all animals of each group, tumor volume (mm³) on the y-axis. Box plots show 10-90 percentile, separate symbols represent values outside this range (outliers), + indicates the mean value. A: vehicle, B. anti-LAG-3, C. 4SC-202, D. anti-PD1 + anti-LAG3, E. anti-PD-1, F. 4SC-202 + anti-LAG3, G. 4SC-202 + anti-PD-1, H. 4SC-202 + anti-PD-1 + anti-LAG3.

FIG. 3: Individual tumor curves, days on the x-axes: days, tumor volume (mm³) on the y-axes. A: vehicle, B. anti-LAG-3, C. 4SC-202, D. anti-PD1 + anti-LAG3, E. anti-PD-1, F. 4SC-202 + anti-LAG3, G. 4SC-202 + anti-PD-1, H. 4SC-202 + anti-PD-1 + anti-LAG3.

### DESCRIPTION OF THE INVENTION

It has now been found unexpectedly that the combination therapy utilizing an HDAC inhibitor of the present invention with a LAG-3 inhibitor and a PD-1 inhibitor or PD-L1 inhibitor shows beneficial efficacy compared with monotherapy.

Certain embodiments of the present invention are listed in the following items:

1. Use of an HDAC inhibitor for the manufacture of a medicament for the treatment of cancer in combination with a LAG-3 inhibitor and a PD-1 inhibitor or PD-L1 inhibitor.

2. The use according to item 1, wherein the HDAC inhibitor is class I HDAC specific.

3. The use according to item 1, wherein the HDAC inhibitor is a molecule of the general formula I

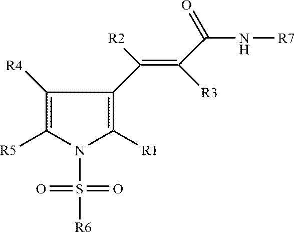

in which
R1, R4 and R5 are independently hydrogen, 1-4C-alkyl, halogen, or 1-4C-alkoxy,
R2 and R3 are independently hydrogen or 1-4C-alkyl,
R6 is -T1 -Q1, in which T1 is a bond or 1-4C-alkylene,
either Q1 is substituted by R61 and/or R62, and is Aa1, Hh1, Ha1, Ha2, Ha3, Ha4 or Ah1, or Q1 is unsubstituted, and is Ha2, Ha3 or Ha4,
in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, -U-T3-N(R613)R614, -T4-Het3, or -V-T5-Het4, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is -O- (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl, 1-4C-alkylcarbonyl, or 1-4C-alkylsulphonyl
R614 is hydrogen or 1-4C-alkyl,
or R613 and R614 together and with inclusion of the nitrogen atom, to which they are bonded, form a heterocyclic ring Het2, in which
Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
T4 is a bond or 1-4C-alkylene,
Het3 is 1N-(1-4C-alkyl)-piperidinyl or 1N-(1-4C-alkyl)-pyrrolidinyl,
V is —O— (oxygen) or —C(O)NH—,
T5 is a bond or 1-4C-alkylene,
Het4 is 1N-(1-4C-alkyl)-piperidinyl or 1N-(1-4C-alkyl)-pyrrolidinyl,
R62 is 1-4C-alkyl, 1-4C-alkoxy or halogen,
Aa1 is a bisaryl radical made up of two aryl groups, which are selected independently from a group consisting of phenyl and naphthyl, and which are linked together via a single bond,
Hh1 is a bisheteroaryl radical made up of two heteroaryl groups, which are selected independently from a group consisting of monocyclic 5- or 6-membered heteroaryl radicals comprising one or two heteroatoms, each of which is selected from the group consisting of nitrogen, oxygen and sulfur, and which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group,
R7 is hydroxyl, or Cyc1, in which Cyc1 is a ring system of formula Ia

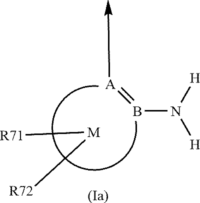

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring, Har2 is a monocyclic 5- or 6-membered unsaturated heteroaromatic ring comprising one to three heteroatoms, each of which is selected from the group consisting of nitrogen, oxygen and sulfur,
or a molecule of the general formula III:

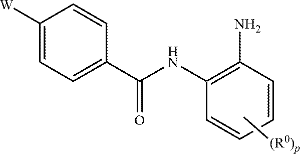

wherein:
R⁰ is halogen;
p is 0, 1, or 2;
W is -NHC(O)(C₁-C₆ alkyl) or —CH₂NH—V; and
V is -CO₂-CH₂-(heteroaryl), -C(O)-CH=CH-(heteroaryl), or heteroaryl optionally substituted by 1-3 heteroaryl groups
or a salt or solvate thereof.

4. The use according to item 3, wherein the HDAC inhibitor is a molecule of the general formula I as detailed in claim 3, or a salt or solvate thereof.

5. The use according to item 1, wherein the HDAC inhibitor is (E)-N-(2-aminophenyl)-3-(1 -((4-(1-methyl-1H-pyrazol-4-yl)phenyl)sulfonyl)-1H-pyrrol-3-yl)acrylamide (also known as 4SC-202) or a salt or solvate thereof.

6. The use according to item 1, wherein the HDAC inhibitor is 4SC-202 tosylate salt.

7. The use according to any one of items 1 to 6, wherein the LAG-3 inhibitor is selected from the group consisting of IMP761 (immutep, formerly prima biomed), IMP701 (immutep, formerly prima biomed), IMP731 (immutep, formerly prima biomed), Sym022 (Symphogen), YBL-011 (Y-Biologics), TJA3 (I-MAB Biopharma), relatlimab (Bristol-Myers Squibb Co), LAG-525 (Novartis AG), REGN-3767 (Regeneron Pharmaceuticals), BI-754111 (Boehringer Ingelheim GmbH), MK-4280 (Merck & Co Inc), TSR-033 (Tesaro Inc), MGD-013 (MacroGenics Inc), AM-0003 (ARMO Biosciences Inc), FS-118 (F-star Biotechnology Ltd), XmAb-22841 (Xencor Inc), and AVA-017 (Avacta Life Sciences Ltd), particularly Sym022 (Symphogen), relatlimab (Bristol-Myers Squibb Co), LAG-525 (Novartis AG), REGN-3767 (Regeneron Pharmaceuticals), BI-754111 (Boehringer Ingelheim GmbH), MK-4280 (Merck & Co Inc), TSR-033 (Tesaro Inc), MGD-013 (MacroGenics Inc), AM-0003 (ARMO Biosciences Inc), FS-118 (F-star Biotechnology Ltd), XmAb-22841 (Xencor Inc), and AVA-017 (Avacta Life Sciences Ltd).

8. The use according to any one of items 1 to 7, wherein the PD-1 inhibitor is selected from the group consisting of pidilizumab (CT-011, CureTech), AMP-224 (Medlmmune), AB122 (Harbin / Wuxi / Arcus), AMP-224 (Medlmmune), MEDI-5752 (Medimmune Oncology Inc), PD1-PIK (Huashan Hospital), PF-06936308 (Pfizer Inc), RG-7769 (Hoffmann-La Roche AG), F-520 (Shandong New Time Pharmaceutical Co Ltd), CAB PD-1 Abs (BioAtla Llc), AK-123 (Akeso Biopharma Inc), MEDI-3387 (Medlmmune LLC), MEDI-5771 (Medlmmune LLC), 4H1128Z-E27 (Eureka Therapeutics Inc; Memorial Sloan-Kettering Cancer Center), REMD-288 (Shandong Danhong Pharmaceutical Co Ltd), SG-001 (Hangzhou Sumgen Biotechnology Co Ltd), BY-24.3 (Beijing Beyond Biotechnology Co Ltd; Hangzhou Sumgen Biotechnology Co Ltd), CB-201 (Crescendo Biologics Ltd), IBI-319 (Adimab LLC; Eli Lilly & Co; Innovent Biologics Inc), ONCR-177 (Oncorus Inc), Max-1 (Maxinovel Pharmaceuticals Inc), CS-4100 (Shenzhen Chipscreen Biosciences Ltd), JBI-426 (Jubilant Biosys Ltd), CCC-0701 (Zenyaku Kogyo Co Ltd), CCX-4503 (ChemoCentryx Inc), nivolumab (BMS), pembrolizumab (Merck), BCD-100 (Biocad), cemiplimab (Regeneron Pharmaceuticals Inc), sintilimab (IBI-308 by Eli Lilly/ Innovent Biologics, Inc.), JNJ-3283 (Johnson & Johnson), spartalizumab (PDR-001 by Novartis AG), camrelizumab (SHR-1210 by Incyte Corp/Jiangsu Hengrui), tislelizumab (BeiGene Ltd), AGEN-2034 (Agenus Inc), MEDI-0680 (AMP-514; Amplimmune/ Medlmmune LLC), toripalimab (JS-001 by Shanghai Junshi Bioscience Co Ltd), dostarlimab (TSR-042 by Tesaro Inc), ABBV-181 (AbbVie Inc), AK-104 (Akeso Biopharma Inc), AK-105 (Akeso Biopharma Inc), BAT-1306 (Bio-Thera Solutions Ltd), Bl-754091 (Boehringer Ingelheim GmbH), CBT-501, Genolimzumab (CBT Pharmaceuticals Inc/Genor), GLS-010 (Harbin Gloria/WuXi/Arcus), LZM-009 (Livzon Pharmaceutical Group Inc), MGA-012 (Incyte Corp/MacroGenics), MGD-013 (MacroGenics Inc), PF-06801591 (Pfizer Inc), Sym-021 (Symphogen A/S), CS-1003 (CStone Pharmaceuticals Co Ltd), HLX-10 (Henlix Biotech / Shanghai Henlius Biotech Co Ltd), AK-103 (Akeso Biopharma Inc), AM-0001 (ARMO Biosciences Inc), TILT-123 (TILT Biotherapeutics Ltd), BH-2922 (Beijing Hanmi Pharmaceutical), BH-2941 (Beijing Hanmi Pharmaceutical), BH-2950 (Beijing Hanmi Pharmaceutical), CX-188 (CytomX Therapeutics Inc), ENUM-244C8 (Enumeral Biomedical Holdings), ENUM-388D4 (Enumeral Biomedical Holdings), HAB-21 (Suzhou Stainwei Biotech Inc), HEISCOIII-003 (Sichuan Haisco Pharmaceutical), IKT-202 (Icell Kealex Therapeutics), JS-003 (Shanghai Junshi Bioscience), JTX-4014 (Jounce Therapeutics Inc), MCLA-134 (Merus NV), MGD-019 (MacroGenics Inc), MT-17000 (Molecular Templates Inc), PEGMP-7 (D5Pharma Inc), PRS-332 (Pieris Pharmaceuticals Inc), RXI-762 (RXi Pharmaceuticals Corp), STI-1110 (Les Laboratoires Servier / Sorrento), VXM-10 (Vaximm AG), XmAb-20717 (Xencor Inc), XmAb-23104 (Xencor Inc), AK-112 (Akeso Biopharma Inc), HLX-20 (Henlix Biotech / Shanghai Henlius Biotech Co Ltd), SSI-361 (Lyvgen Biopharma Ltd), AT-16201 (AIMM Therapeutics BV), and SNA-01 (Fountain Biopharma Inc)..

9. The use according to any one of items 1 to 7, wherein the PD-L1 inhibitor is selected from the group consisting of BCD-135 (Biocad), APL-502 (CBT-402 or TQB2450, Apollomics), MDX-1 105 (BMS-936559, Bristol-Myers Squibb), IMC-001 (ImmuneOncia Therapeutics LLC; Sorrento Therapeutics Inc), KD-045 (Nanjing Kaedi Biotech Inc), INBRX-105 (Elpiscience Biopharmaceuticals Inc; INHIBRx LLC), KN-046 (Suzhou Alphamab Co Ltd), INCB-086550 (Incyte Corp), IMC-2102 (ImmuneOncia Therapeutics LLC), IMC-2101 (ImmuneOncia Therapeutics LLC), anti-PDL1-TGFbRllecd (Johns Hopkins University; Y-Trap Inc), KD-005 (Nanjing Kaedi Biotech Inc), PM-PDL-GEX (Glycotope GmbH), IMM-2502 (ImmuneOnco Biopharm Co Ltd), 89Zr-CX-072 (CytomX Therapeutics Inc; University Medical Center Groningen), KY-1055 (Kymab Ltd), MEDI-1109 (Medlmmune LLC), MT-5594 (Molecular Templates Inc), 89Zr-DFO-6E11 (Minerva Imaging; Thermo Fisher Scientific Inc), SL-279252 (Shattuck Labs Inc; Takeda Pharmaceutical Co Ltd), DSP-106 (KAHR Medical Ltd; University Medical Center Groningen), Gensci-047 (GeneScience Pharmaceuticals Co Ltd), REMD-290 (Shandong Danhong Pharmaceutical Co Ltd), N-809 (NantKwest Inc; National Cancer Institute), PRS-344 (Pieris Pharmaceuticals Inc; Servier), FS-222 (F-star Biotechnologische Forschungs-und Entwicklungs GmbH), GEN-1046 (BioNTech SE; Genmab Holding BV), BH-29xx (Beijing Hanmi Pharmaceutical Co Ltd), atezolizumab (Genentech Inc), avelumab (Merck KGaA/Pfizer), durvalumab (AstraZeneca Pharmaceuticals LP/Medlmmune), BGB-A333 (BeiGene Ltd), CX-072 (CytomX Therapeutics Inc), GNS-1480 (Yuhan Corp/Genosco), AMP-224 (Medlmmune LLC), CA-170 (Aurigene Discovery Technologies Ltd), CK-301 (Checkpoint Therapeutics/TG Therapeutics), CS-1001 (CStone Pharmaceuticals Co Ltd), FAZ-053 (Novartis AG), envafolimab (ASC22 or KN-035; Suzhou AlphaMab/3DMed), LY-3300054 (Eli Lilly and Co), M-7824 (Merck KGaA), HTI-1088 (HTI-131, SHR-1316; Atridia and Jiangsu Hengrui Medicine Co.), MSB-2311 (MabSpace Biosciences (Suzhou) Co Ltd), STI-A1014 (Lee’s Pharmaceutical/Sorrento), AK-106 (Akeso Biopharma Inc), AVA-004 (Avacta Life Sciences Ltd), BBI-801 (Boston Biomedical Inc), CA-327 (Aurigene/Curis), CBA-0710 (Sorrento Therapeutics Inc), CBT-502 (CBT Pharmaceuticals/Chia Tai Tianqing Pharmaceutical), FPT-155 (Five Prime Therapeutics Inc), FS-118 (F-star Biotechnology Ltd), IKT-201 (Icell Kealex Therapeutics), IKT-703 (Icell Kealex Therapeutics), IO-103 (IO Biotech ApS), JS-003 (Shanghai Junshi Bioscience), KD-033 (Kadmon Corp/Jinghua Pharmaceutical), KY-1003 (Kymab Ltd), MCLA-145 (Merus NV/Incyte), MT-5050 (Molecular Templates Inc), and SNA-02 (Fountain Biopharma Inc)..

10. The use according to any one of items 1 to 9, wherein said cancer is selected from the group consisting of melanoma (in particular ocular and uveal, but also including skin melanoma), head and neck, renal, Non-small cell lung cancer (NSCLC), microsatellite-instable carcinoma (lynch syndrome, in particular gastroesophageal and colorectal), urothelial carcinoma including bladder cancer, merkel cell carcinoma, hodgkin lymphoma, gastric, gastrointestinal cancers (microsatellite stable and instable) including colorectal cancer (CRC), hepatocellular carcinoma (HCC), renal cell carcinoma (RCC), nasopharyngeal, basal cell carcinoma, cervical cancer, anogenital cancers, Kaposi sarcoma, adult T-cell leukemia, primary effusion lymphoma, and Castlemann’s disease, or selected from the group consisting of breast cancer, in particular triple-negative breast cancer, oesophageal cancer, non-hodgkin lymphoma, small cell lung cancer (SCLC), sarkoma, mesothelioma, glioblastoma, microsatellite stable cancer (in particular gastroesophageal and colorectal), pancreas cancer, prostate cancer, cutaneous T-cell lymphoma (CTCL), and squamous cell carcinoma.

In certain embodiments (E)-N-(2-aminophenyl)-3-(1-((4-(1-methyl-1H-pyrazol-4-yl)phenyl)sulfonyl)-1H-pyrrol-3-yl)acrylamide is administered in a dose of 80 to 120, particularly 90 to 110, more particularly 95 to 105, even more particularly about 100 mg/day, or alternatively twice any of the aforementioned doses, or alternatively 4 times any of the aforementioned doses, wherein any of said daily doses are optionally administered in two portions (each half of the aforementioned amounts), twice daily, particularly one each in the morning and evening (wherein particularly the evening dose is administered 10-14, more particularly 11-13, even more particularly about 12 hours after the morning dose).

In certain embodiments, the LAG-3 inhibitor and/or the PD-1 inhibitor and/or the PD-L1 inhibitor may be selected from the group consisting of an antibody, e.g. a monoclonal or bispecific monoclonal antibody, a fusion protein, a recombinant protein, or an aptamer, an oncolytic virus, an antisense RNAi oligonucleotide, a vaccine; particularly an antibody, e.g. a monoclonal or bispecific monoclonal antibody, a fusion protein, a recombinant protein, or an aptamer; more particularly an antibody, e.g. a monoclonal or bispecific monoclonal antibody; and in certain embodiments, biosimilars of the aforementioned, in particular of antibodies are encompassed by the present invention.

The LAG-3 inhibitor and/or the PD-1 inhibitor and/or the PD-L1 inhibitor may be specific for these respective targets, or they may act on two or more targets, which may include acting on PD-1 and LAG-3 or PD-L1 and LAG-3 (in which cases a single molecule, e.g. a bispecific antibody, would suffice to the criteria of being a “LAG-3 inhibitor and a PD-1 inhibitor or PD-L1 inhibitor”), or may include, in addition to activity on PD-1, PD-L1 or LAG-3, activity on one or more further targets.

In particular embodiments, the LAG-3 inhibitor is selected from the group consisting of relatlimab (Bristol-Myers Squibb Co), LAG-525 (Novartis AG), REGN-3767 (Regeneron Pharmaceuticals), BI-754111 (Boehringer Ingelheim GmbH), MK-4280 (Merck & Co Inc), TSR-033 (Tesaro Inc), MGD-013 (MacroGenics Inc), AM-0003 (ARMO Biosciences Inc), FS-118 (F-star Biotechnology Ltd), XmAb-22841 (Xencor Inc), and AVA-017 (Avacta Life Sciences Ltd); more particularly relatlimab, LAG-525, REGN-3767, BI-754111, MK-4280, TSR-033, and MGD-013.

In other particular embodiments, the LAG-3 inhibitor is selected from the group consisting of IMP761(immutep, formerly prima biomed), IMP701(immutep, formerly prima biomed), IMP731 (immutep, formerly prima biomed), Sym022 (Symphogen), YBL-011 (Y-Biologics), TJA3 (I-MAB Biopharma), relatlimab (Bristol-Myers Squibb Co), LAG-525 (Novartis AG), REGN-3767 (Regeneron Pharmaceuticals), BI-754111 (Boehringer Ingelheim GmbH), MK-4280 (Merck & Co Inc), TSR-033 (Tesaro Inc), MGD-013 (MacroGenics Inc), AM-0003 (ARMO Biosciences Inc), FS-118 (F-star Biotechnology Ltd), XmAb-22841 (Xencor Inc), and AVA-017 (Avacta Life Sciences Ltd); more particularly Sym022 (Symphogen), relatlimab, LAG-525, REGN-3767, BI-754111, MK-4280, TSR-033, and MGD-013.

In particular embodiments the PD-1 inhibitor is selected from the group consisting of nivolumab (BMS), pembrolizumab (Merck), BCD-100 (Biocad), cemiplimab (Regeneron Pharmaceuticals Inc), sintilimab (IBI-308 by Eli Lilly / Innovent Biologics, Inc.), JNJ-3283 (Johnson & Johnson), spartalizumab (PDR-001 by Novartis AG), camrelizumab (SHR-1210 by Incyte Corp/Jiangsu Hengrui), tislelizumab (BeiGene Ltd), AGEN-2034 (Agenus Inc), MEDI-0680 (AMP-514; Amplimmune / Medlmmune LLC), toripalimab (JS-001 by Shanghai Junshi Bioscience Co Ltd), dostarlimab (TSR-042 by Tesaro Inc), ABBV-181 (AbbVie Inc), AK-104 (Akeso Biopharma Inc), AK-105 (Akeso Biopharma Inc), BAT-1306 (Bio-Thera Solutions Ltd), BI-754091 (Boehringer Ingelheim GmbH), CBT-501, Genolimzumab (CBT Pharmaceuticals Inc/Genor), GLS-010 (Harbin Gloria/WuXi/Arcus), LZM-009 (Livzon Pharmaceutical Group Inc), MGA-012 (Incyte Corp/MacroGenics), MGD-013 (MacroGenics Inc), PF-06801591 (Pfizer Inc), Sym-021 (Symphogen A/S), CS-1003 (CStone Pharmaceuticals Co Ltd), HLX-10 (Henlix Biotech/ Shanghai Henlius Biotech Co Ltd), AK-103 (Akeso Biopharma Inc), AM-0001 (ARMO Biosciences Inc), TILT-123 (TILT Biotherapeutics Ltd), BH-2922 (Beijing Hanmi Pharmaceutical), BH-2941 (Beijing Hanmi Pharmaceutical), BH-2950 (Beijing Hanmi Pharmaceutical), CX-188 (CytomX Therapeutics Inc), ENUM-244C8 (Enumeral Biomedical Holdings), ENUM-388D4 (Enumeral Biomedical Holdings), HAB-21 (Suzhou Stainwei Biotech Inc), HEISCOIII-003 (Sichuan Haisco Pharmaceutical), IKT-202 (Icell Kealex Therapeutics), JS-003 (Shanghai Junshi Bioscience), JTX-4014 (Jounce Therapeutics Inc), MCLA-134 (Merus NV), MGD-019 (MacroGenics Inc), MT-17000 (Molecular Templates Inc), PEGMP-7 (D5Pharma Inc), PRS-332 (Pieris Pharmaceuticals Inc), RXI-762 (RXi Pharmaceuticals Corp), STI-1110 (Les Laboratoires Servier / Sorrento), VXM-10 (Vaximm AG), XmAb-20717 (Xencor Inc), XmAb-23104 (Xencor Inc), AK-112 (Akeso Biopharma Inc), HLX-20 (Henlix Biotech/ Shanghai Henlius Biotech Co Ltd), SSI-361 (Lyvgen Biopharma Ltd), AT-16201 (AIMM Therapeutics BV), and SNA-01 (Fountain Biopharma Inc); more particularly nivolumab, pembrolizumab, BCD-100, cemiplimab, IBI-308, JNJ-3283, PDR-001, SHR-1210, tislelizumab, AGEN-2034, MEDI-0680, JS-001, TSR-042, ABBV-181, AK-104, AK-105, BAT-1306, Bl-754091, CBT-501, Genolimzumab, GLS-010, LZM-009, MGA-012, MGD-013, PF-06801591, Sym-021, CS-1003, and HLX-10; even more particularly nivolumab, pembrolizumab, BCD-100, cemiplimab, IBI-308, JNJ-3283, PDR-001, SHR-1210, tislelizumab, AGEN-2034, MEDI-0680, JS-001, and TSR-042, yet even more particularly nivolumab, pembrolizumab, BCD-100, cemiplimab, IBI-308, JNJ-3283, PDR-001, SHR-1210, and tislelizumab; yet even more particularly nivolumab and pembrolizumab.

In other particular embodiments the PD-1 inhibitor is selected from the group consisting of Pidilizumab (CT-011, CureTech), AMP-224 (MedImmune), AB122 (Harbin / Wuxi / Arcus), AMP-224 (MedImmune), MEDI-5752 (MedImmune Oncology Inc), PD1-PIK (Huashan Hospital), PF-06936308 (Pfizer Inc), RG-7769 (Hoffmann-La Roche AG), F-520 (Shandong New Time Pharmaceutical Co Ltd), CAB PD-1 Abs (BioAtla Llc), AK-123 (Akeso Biopharma Inc), MEDI-3387 (MedImmune LLC), MEDI-5771 (MedImmune LLC), 4H1128Z-E27 (Eureka Therapeutics Inc; Memorial Sloan-Kettering Cancer Center), REMD-288 (Shandong Danhong Pharmaceutical Co Ltd), SG-001 (Hangzhou Sumgen Biotechnology Co Ltd), BY-24.3 (Beijing Beyond Biotechnology Co Ltd; Hangzhou Sumgen Biotechnology Co Ltd), CB-201 (Crescendo Biologics Ltd), IBI-319 (Adimab LLC; Eli Lilly & Co; Innovent Biologics Inc), ONCR-177 (Oncorus Inc), Max-1 (Maxinovel Pharmaceuticals Inc), CS-4100 (Shenzhen Chipscreen Biosciences Ltd), JBI-426 (Jubilant Biosys Ltd), CCC-0701 (Zenyaku Kogyo Co Ltd), CCX-4503 (ChemoCentryx Inc), nivolumab (BMS), pembrolizumab (Merck), BCD-100 (Biocad), cemiplimab (Regeneron Pharmaceuticals Inc), sintilimab (IBI-308 by Eli Lilly / Innovent Biologics, Inc.), JNJ-3283 (Johnson & Johnson), spartalizumab (PDR-001 by Novartis AG), camrelizumab (SHR-1210 by Incyte Corp/Jiangsu Hengrui), tislelizumab (BeiGene Ltd), AGEN-2034 (Agenus Inc), MEDI-0680 (AMP-514; Amplimmune / MedImmune LLC), toripalimab (JS-001 by Shanghai Junshi Bioscience Co Ltd), dostarlimab (TSR-042 by Tesaro Inc), ABBV-181 (AbbVie Inc), AK-104 (Akeso Biopharma Inc), AK-105 (Akeso Biopharma Inc), BAT-1306 (Bio-Thera Solutions Ltd), BI-754091 (Boehringer Ingelheim GmbH), CBT-501, Genolimzumab (CBT Pharmaceuticals Inc/Genor), GLS-010 (Harbin Gloria/WuXi/Arcus), LZM-009 (Livzon Pharmaceutical Group Inc), MGA-012 (Incyte Corp/MacroGenics), MGD-013 (MacroGenics Inc), PF-06801591 (Pfizer Inc), Sym-021 (Symphogen A/S), CS-1003 (CStone Pharmaceuticals Co Ltd), HLX-10 (Henlix Biotech / Shanghai Henlius Biotech Co Ltd), AK-103 (Akeso Biopharma Inc), AM-0001 (ARMO Biosciences Inc), TILT-123 (TILT Biotherapeutics Ltd), BH-2922 (Beijing Hanmi Pharmaceutical), BH-2941 (Beijing Hanmi Pharmaceutical), BH-2950 (Beijing Hanmi Pharmaceutical), CX-188 (CytomX Therapeutics Inc), ENUM-244C8 (Enumeral Biomedical Holdings), ENUM-388D4 (Enumeral Biomedical Holdings), HAB-21 (Suzhou Stainwei Biotech Inc), HEISCOIII-003 (Sichuan Haisco Pharmaceutical), IKT-202 (Icell Kealex Therapeutics), JS-003 (Shanghai Junshi Bioscience), JTX-4014 (Jounce Therapeutics Inc), MCLA-134 (Merus NV), MGD-019 (MacroGenics Inc), MT-17000 (Molecular Templates Inc), PEGMP-7 (D5Pharma Inc), PRS-332 (Pieris Pharmaceuticals Inc), RXI-762 (RXi Pharmaceuticals Corp), STI-1110 (Les Laboratoires Servier / Sorrento), VXM-10 (Vaximm AG), XmAb-20717 (Xencor Inc), XmAb-23104 (Xencor Inc), AK-112 (Akeso Biopharma Inc), HLX-20 (Henlix Biotech / Shanghai Henlius Biotech Co Ltd), SSI-361 (Lyvgen Biopharma Ltd), AT-16201 (AIMM Therapeutics BV), and SNA-01 (Fountain Biopharma Inc); more particularly sintilimab, spartalizumab, camrelizumab, toripalimab, pidilizumab, AMP-224, AB122, AMP-224, dostarlimab, MEDI-5752, PD1-PIK, PF-06936308, RG-7769, nivolumab, pembrolizumab, BCD-100, cemiplimab, IBI-308, JNJ-3283, PDR-001, SHR-1210, tislelizumab, AGEN-2034, MEDI-0680, JS-001, TSR-042, ABBV-181, AK-104, AK-105, BAT-1306, BI-754091, CBT-501, Genolimzumab, GLS-010, LZM-009, MGA-012, MGD-013, PF-06801591, Sym-021, CS-1003, and HLX-10; even more particularly sintilimab, spartalizumab, camrelizumab, toripalimab, pidilizumab, AMP-224, AB122, AMP-224, dostarlimab, MEDI-5752, PD1-PIK, PF-06936308, RG-7769, nivolumab, pembrolizumab, BCD-100, cemiplimab, IBI-308, JNJ-3283, PDR-001, SHR-1210, tislelizumab, AGEN-2034, MEDI-0680, JS-001, and TSR-042, yet even more particularly sintilimab, spartalizumab, camrelizumab, toripalimab, pidilizumab, dostarlimab, nivolumab, pembrolizumab, BCD-100, cemiplimab, IBI-308, JNJ-3283, PDR-001, SHR-1210, and tislelizumab; yet even more particularly nivolumab and pembrolizumab.

In particular embodiments the PD-L1 inhibitor is selected from the group consisting of atezolizumab (Genentech Inc), avelumab (Merck KGaA/Pfizer), durvalumab (AstraZeneca Pharmaceuticals LP/Medlmmune), BGB-A333 (BeiGene Ltd), CX-072 (CytomX Therapeutics Inc), GNS-1480 (Yuhan Corp/Genosco), AMP-224 (Medlmmune LLC), CA-170 (Aurigene Discovery Technologies Ltd), CK-301 (Checkpoint Therapeutics/TG Therapeutics), CS-1001 (CStone Pharmaceuticals Co Ltd), FAZ-053 (Novartis AG), envafolimab (ASC22 or KN-035; Suzhou AlphaMab/3DMed), LY-3300054 (Eli Lilly and Co), M-7824 (Merck KGaA), HTI-1088 (HTI-131, SHR-1316; Atridia and Jiangsu Hengrui Medicine Co.), MSB-2311 (MabSpace Biosciences (Suzhou) Co Ltd), STI-A1014 (Lee’s Pharmaceutical/Sorrento), AK-106 (Akeso Biopharma Inc), AVA-004 (Avacta Life Sciences Ltd), BBI-801 (Boston Biomedical Inc), CA-327 (Aurigene/Curis), CBA-0710 (Sorrento Therapeutics Inc), CBT-502 (CBT Pharmaceuticals/Chia Tai Tianqing Pharmaceutical), FPT-155 (Five Prime Therapeutics Inc), FS-118 (F-star Biotechnology Ltd), IKT-201 (Icell Kealex Therapeutics), IKT-703 (Icell Kealex Therapeutics), IO-103 (IO Biotech ApS), JS-003 (Shanghai Junshi Bioscience), KD-033 (Kadmon Corp/Jinghua Pharmaceutical), KY-1003 (Kymab Ltd), MCLA-145 (Merus NV/Incyte), MT-5050 (Molecular Templates Inc), and SNA-02 (Fountain Biopharma Inc); more particularly atezolizumab, avelumab, durvalumab, BGB-A333, CX-072, GNS-1480, AMP-224, CA-170, CK-301, CS-1001, FAZ-053, KN-035, LY-3300054, M-7824, SHR-1316, MSB-2311, and STI-A1014; even more particularly atezolizumab, avelumab, durvalumab, BGB-A333, CX-072, and GNS-1480; yet even more particularly atezolizumab, avelumab, and durvalumab.

In other particular embodiments the PD-L1 inhibitor is selected from the group consisting of BCD-135 (Biocad), APL-502 (CBT-402 or TQB2450, Apollomics), MDX-1105 (BMS-936559, Bristol-Myers Squibb), IMC-001 (ImmuneOncia Therapeutics LLC; Sorrento Therapeutics Inc), KD-045 (Nanjing Kaedi Biotech Inc), INBRX-105 (Elpiscience Biopharmaceuticals Inc; INHIBRx LLC), KN-046 (Suzhou Alphamab Co Ltd), INCB-086550 (Incyte Corp), IMC-2102 (ImmuneOncia Therapeutics LLC), IMC-2101 (ImmuneOncia Therapeutics LLC), anti-PDL1-TGFbRllecd (Johns Hopkins University; Y-Trap Inc), KD-005 (Nanjing Kaedi Biotech Inc), PM-PDL-GEX (Glycotope GmbH), IMM-2502 (ImmuneOnco Biopharm Co Ltd), 89Zr-CX-072 (CytomX Therapeutics Inc; University Medical Center Groningen), KY-1055 (Kymab Ltd), MEDI-1 109 (Medlmmune LLC), MT-5594 (Molecular Templates Inc), 89Zr-DFO-6E11 (Minerva Imaging; Thermo Fisher Scientific Inc), SL-279252 (Shattuck Labs Inc; Takeda Pharmaceutical Co Ltd), DSP-106 (KAHR Medical Ltd; University Medical Center Groningen), Gensci-047 (GeneScience Pharmaceuticals Co Ltd), REMD-290 (Shandong Danhong Pharmaceutical Co Ltd), N-809 (NantKwest Inc; National Cancer Institute), PRS-344 (Pieris Pharmaceuticals Inc; Servier), FS-222 (F-star Biotechnologische Forschungs-und Entwicklungs GmbH), GEN-1046 (BioNTech SE; Genmab Holding BV), BH-29xx (Beijing Hanmi Pharmaceutical Co Ltd), atezolizumab (Genentech Inc), avelumab (Merck KGaA/Pfizer), durvalumab (AstraZeneca Pharmaceuticals LP/Medlmmune), BGB-A333 (BeiGene Ltd), CX-072 (CytomX Therapeutics Inc), GNS-1480 (Yuhan Corp/Genosco), AMP-224 (Medlmmune LLC), CA-170 (Aurigene Discovery Technologies Ltd), CK-301 (Checkpoint Therapeutics/TG Therapeutics), CS-1001 (CStone Pharmaceuticals Co Ltd), FAZ-053 (Novartis AG), envafolimab (ASC22 or KN-035; Suzhou AlphaMab/3DMed), LY-3300054 (Eli Lilly and Co), M-7824 (Merck KGaA), HTI-1088 (HTI-131, SHR-1316; Atridia and Jiangsu Hengrui Medicine Co.), MSB-2311 (MabSpace Biosciences (Suzhou) Co Ltd), STI-A1014 (Lee’s Pharmaceutical/Sorrento), AK-106 (Akeso Biopharma Inc), AVA-004 (Avacta Life Sciences Ltd), BBI-801 (Boston Biomedical Inc), CA-327 (Aurigene/Curis), CBA-0710 (Sorrento Therapeutics Inc), CBT-502 (CBT Pharmaceuticals/Chia Tai Tianqing Pharmaceutical), FPT-155 (Five Prime Therapeutics Inc), FS-118 (F-star Biotechnology Ltd), IKT-201 (Icell Kealex Therapeutics), IKT-703 (Icell Kealex Therapeutics), IO-103 (IO Biotech ApS), JS-003 (Shanghai Junshi Bioscience), KD-033 (Kadmon Corp/Jinghua Pharmaceutical), KY-1003 (Kymab Ltd), MCLA-145 (Merus NV/Incyte), MT-5050 (Molecular Templates Inc), and SNA-02 (Fountain Biopharma Inc); more particularly BCD-135, APL-502, MDX-1105, IMC-001, KD-045, INBRX-105, KN-046, INCB-086550, envafolimab, atezolizumab, avelumab, durvalumab, BGB-A333, CX-072, GNS-1480, AMP-224, CA-170, CK-301, CS-1001, FAZ-053, KN-035, LY-3300054, M-7824, SHR-1316, MSB-2311, and STI-A1014; even more particularly envafolimab, atezolizumab, avelumab, durvalumab, BGB-A333, CX-072, and GNS-1480; yet even more particularly atezolizumab, avelumab, and durvalumab.

In further embodiments, the PD-1 or PD-L1 inhibitor is a monoclonal antibody or antigen-binding fragment thereof comprising defined sequences of complementarity determining regions (CDRs) or of variable regions of heavy and light chains, such as the ones listed below. The abYsis integrated database and analysis suite (version 3.1.0) and Kabat numbering system were used for annotation of sequences.

| Region | Sequence |
|---|---|
| | Anti-PD-1 Antibody 1 |
| CDRL1 | RASKGVSTSGYSYLH (SEQ ID NO: 1) |
| CDRL2 | LASYLES (SEQ ID NO: 2) |
| CDRL3 | QHSRDLPLT (SEQ ID NO: 3) |
| CDRH1 | NYYMY (SEQ ID NO: 4) |
| CDRH2 | GINPSNGGTNFNEKFKN (SEQ ID NO: 5) |
| CDRH3 | RDYRFDMGFDY (SEQ ID NO: 6) |
| LCVR | EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK (SEQ ID NO: 7) |
| HCVR | QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS (SEQ ID NO: 8) |
| LC | EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC (SEQ ID NO: 9) |
| HC | QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK (SEQ ID NO: 10) |

| | Anti-PD-1 Antibody 2 |
|---|---|
| CDRL1 | RASQSVSSYLAW (SEQ ID NO: 11) |
| CDRL2 | DASNRAT (SEQ ID NO: 12) |
| CDRL3 | QQSSNWPRT (SEQ ID NO: 13) |
| CDRH1 | SNSGMH (SEQ ID NO: 14) |
| CDRH2 | VIWYDGSKRYYADSVKG (SEQ ID NO: 15) |
| CDRH3 | NDDY (SEQ ID NO: 16) |
| LCVR | EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIK (SEQ ID NO: 17) |
| HCVR | QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSS (SEQ ID NO: 18) |
| LC | EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC (SEQ ID NO: 19) |
| HC | QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK (SEQ ID NO: 20) |

| | Anti-PD-1 Antibody 3 |
|---|---|
| CDRL1 | RASLSINTFLN (SEQ ID NO: 21) |
| CDRL2 | AASSLHG (SEQ ID NO: 22) |
| CDRL3 | QQSSNTPFT (SEQ ID NO: 23) |
| CDRH1 | NFGMT (SEQ ID NO: 24) |
| CDRH2 | GISGGGRDTYFADSVKG (SEQ ID NO: 25) |
| CDRH3 | WGNIYFDY (SEQ ID NO: 26) |
| LCVR | DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFR (SEQ ID NO: 27) |
| HCVR | EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSS (SEQ ID NO: 28) |
| LC | DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC (SEQ ID NO: 29) |
| HC | EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDS DGSFFLYSRLTVDKSRWQEGNVFSCSVMHEALHNHYTQKSLSLSLGK (SEQ ID NO: 30) |

| | Anti-PD-L1 Antibody 1 |
|---|---|
| CDRL1 | RASQDVSTAVA (SEQ ID NO: 31) |
| CDRL2 | SASFLYS (SEQ ID NO: 32) |
| CDRL3 | QQYLYHPAT (SEQ ID NO: 33) |
| CDRH1 | DSWIH (SEQ ID NO: 34) |
| CDRH2 | WISPYGGSTYYADSVKG (SEQ ID NO: 35) |
| CDRH3 | RHWPGGFDY (SEQ ID NO: 36) |
| LCVR | DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK (SEQ ID NO: 37) |
| HCVR | EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSS (SEQ ID NO: 38) |
| LC | DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC (SEQ ID NO: 39) |
| HC | EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK (SEQ ID NO: 40) |

| | Anti-PD-L1 Antibody 2 |
|---|---|
| CDRL1 | TGTSSDVGGYNYVS (SEQ ID NO: 41) |
| CDRL2 | DVSNRPS (SEQ ID NO: 42) |
| CDRL3 | SSYTSSSTRV (SEQ ID NO: 43) |
| CDRH1 | SYIMM (SEQ ID NO: 44) |
| CDRH2 | SIYPSGGITFYADTVKG (SEQ ID NO: 45) |
| CDRH3 | IKLGTVTTVDY (SEQ ID NO: 46) |
| LCVR | QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL (SEQ ID NO: 47) |
| HCVR | EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS (SEQ ID NO: 48) |
| LC | QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS (SEQ ID NO: 49) |
| HC | EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK (SEQ ID NO: 50) |

| | Anti-PD-L1 Antibody 3 |
|---|---|
| CDRL1 | RASQRVSSSYLA (SEQ ID NO: 51) |
| CDRL2 | DASSRAT (SEQ ID NO: 52) |
| CDRL3 | QQYGSLPWT (SEQ ID NO: 53) |
| CDRH1 | RYWMS (SEQ ID NO: 54) |
| CDRH2 | NIKQDGSEKYYVDSVKG (SEQ ID NO: 55) |
| CDRH3 | EGGWFGELAFDY (SEQ ID NO: 56) |
| LCVR | EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK (SEQ ID NO: 57) |
| HCVR | EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS (SEQ ID NO: 58) |
| LC | EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC (SEQ ID NO: 59) |
| HC | EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK (SEQ ID NO: 60) |

For instance in some embodiments, the anti-PD-1 monoclonal antibody or PD-1-binding fragment thereof comprises (i) light chain CDR sequences of SEQ ID NOs:1-3 and heavy chain CDR sequences of SEQ ID NOs:4-6, or (ii) a light chain variable region sequence of SEQ ID NO:7 and a heavy chain variable region sequence of SEQ ID NO:8. In some embodiments, the anti-PD-1 monoclonal antibody comprises the light chain sequence of SEQ ID NO:9 and the heavy chain sequence of SEQ ID NO:10. In other embodiments, the anti-PD-1 monoclonal antibody or PD-1-binding fragment thereof comprises (i) light chain CDR sequences of SEQ ID NOs:11-13 and heavy chain CDR sequences of SEQ ID NOs:14-16, or (ii) a light chain variable region sequence of SEQ ID NO:17 and a heavy chain variable region sequence of SEQ ID NO:18. In some embodiments, the anti-PD-1 monoclonal antibody comprises the light chain sequence of SEQ ID NO:19 and the heavy chain sequence of SEQ ID NO:20. In other embodiments, the anti-PD-1 monoclonal antibody or PD-1-binding fragment thereof comprises (i) light chain CDR sequences of SEQ ID NOs:21-23 and heavy chain CDR sequences of SEQ ID NOs:24-26, or (ii) a light chain variable region sequence of SEQ ID NO:27 and a heavy chain variable region sequence of SEQ ID NO:28. In some embodiments, the anti-PD-1 monoclonal antibody comprises the light chain sequence of SEQ ID NO:29 and the heavy chain sequence of SEQ ID NO:30. In other embodiments, the anti-PD-L1 monoclonal antibody or PD-L1-binding fragment thereof comprises (i) light chain CDR sequences of SEQ ID NOs:31-33 and heavy chain CDR sequences of SEQ ID NOs:34-36, or (ii) a light chain variable region sequence of SEQ ID NO:37 and a heavy chain variable region sequence of SEQ ID NO:38. In some embodiments, the anti-PD-L1 monoclonal antibody comprises the light chain sequence of SEQ ID NO:39 and the heavy chain sequence of SEQ ID NO:40. In other embodiments, the anti-PD-L1 monoclonal antibody or PD-L1-binding fragment thereof comprises (i) light chain CDR sequences of SEQ ID NOs:41-43 and heavy chain CDR sequences of SEQ ID NOs:44-46, or (ii) a light chain variable region sequence of SEQ ID NO:47 and a heavy chain variable region sequence of SEQ ID NO:48. In some embodiments, the anti-PD-L1 monoclonal antibody comprises the light chain sequence of SEQ ID NO:49 and the heavy chain sequence of SEQ ID NO:50. In other embodiments, the anti-PD-L1 monoclonal antibody or PD-L1-binding fragment thereof comprises (i) light chain CDR sequences of SEQ ID NOs:51-53 and heavy chain CDR sequences of SEQ ID NOs:54-56, or (ii) a light chain variable region sequence of SEQ ID NO:57 and a heavy chain variable region sequence of SEQ ID NO:58. In some embodiments, the anti-PD-L1 monoclonal antibody comprises the light chain sequence of SEQ ID NO:59 and the heavy chain sequence of SEQ ID NO:60.

In other embodiments, the anti-PD-1 monoclonal antibody comprises CDR sequences identified using the abYsis integrated database and analysis suite (version 3.1.0) and Clothia numbering system of one of the following pairs of light chain and heavy chain sequences: (i) SEQ ID NO:9 and SEQ ID NO:10; (ii) SEQ ID NO:19 and SEQ ID NO:20; or (iii) SEQ ID NO:29 and SEQ ID NO:30. In further embodiments, the anti-PD-L1 monoclonal antibody comprises CDR sequences identified using the abYsis integrated database and analysis suite (version 3.1.0) and Clothia numbering system of one of the following pairs of light chain and heavy chain sequences: (i) SEQ ID NO:39 and SEQ ID NO:40; (ii) SEQ ID NO:49 and SEQ ID NO:50; or (iii) SEQ ID NO:59 and SEQ ID NO:60.

In other embodiments, when the immune checkpoint inhibitor is a monoclonal antibody, the monoclonal antibody may be a variant comprising heavy chain and light chain sequences that are identical to one of the anti-PD-1 or anti-PD-1L monoclonal antibodies named in the preceding paragraphs (reference monoclonal antibodies). The variant is identical to a reference monoclonal antibody, except for having three, two or one conservative amino acid substitutions at positions that are located outside of the light chain complementarity determining regions (CDRs) and six, five, four, three, two or one conservative amino acid substitutions that are located outside of the heavy chain CDRs (e.g., the variant positions are located in the framework regions or the constant region). In other words, the reference monoclonal antibody and the variant monoclonal antibody comprise identical CDR sequences, but differ from each other due to having a conservative amino acid substitution at no more than three or six other positions in their full length light and heavy chain sequences, respectively. The variant monoclonal antibody is substantially the same as the reference monoclonal antibody with respect to the following properties: binding affinity to its target molecule and ability to block the binding of the target molecule to a ligand.

For sake of brevity, in the following descriptions of certain embodiments, LAG-3, PD-1 and PD-L1 are in several cases addressed commonly as “present immune checkpoint”, and consequently, the corresponding inhibitors are commonly addressed as “present immune checkpoint inhibitors”. This generally is to be understood to relate to, as the case may be, either one, two of, or all three of LAG-3, PD-1 and PD-L1 (inhibitors), unless expressly stated to the contrary, and regardless of whether the terms are used in their singular or plural form.

In certain embodiments, the present immune checkpoint inhibitors are molecule for which binding to, as the case may be, LAG-3, PD-1 or PD-L1, respectively, is determinable in an ELISA assay, e.g. an anti-LAG-3 antibody, anti-PD-1 antibody or PD-L1 antibody, in particular with an EC₅₀ 250 nM or lower, more particularly 100 nM or lower, even more particularly 75 nM or lower. A particular ELISA useable in this context, in particular for biologicals, more particularly for antibodies, is the following assays:

Material & Methods:
Present immune checkpoint inhibitor (anti-AG3, or anti-PD-1, or anti-PD-L1)
Recombinant present immune checkpoint, extracellular part of present immune checkpoint or chimeric proteins comprising extracellular part of present immune checkpoint (particularly human)
antibody specific to present immune checkpoint inhibitor, coupled to horse radish peroxidase (HRP)
ELISA Plate 96Well high binding (Greiner #655061)
PBS (e.g. Gibco #21300-058)
BSA (e.g. Sigma #A3733)
Tween20 (e.g. Sigma #P1379)
TMB (3,3′,5,5′-Tetramethylbenzidin) ELISA substrate (e.g. 1-Step™ Ultra TMB-ELISA Substrate Solution by Thermo #34029)
Blocking solution: 1 % BSA in PBS
Washing solution: PBS with 0.05% Tween
Stopping solution: sulphuric acid 250 mM

1. dissolve 0.1 - 1 µg/mL recombinant present immune checkpoint, extracellular part of present immune checkpoint or chimeric proteins comprising extracellular part of present immune checkpoint in PBS, add 100 µL of said solution per well to ELISA 96 well plates, incubate the plates for 12-24 h at 4° C. (to coat the wells with present immune checkpoint)
2. subsequently, remove solution and wash each well twice with 200 µL washing solution
3. subsequently add 200 µL of blocking solution per well, incubate at room temperature (about 22° C.) for 1 hour
4. subsequently, remove blocking solution and wash each well with 1 x 200 µL washing solution
5. subsequently, add 100 µL serial dilutions of present immune checkpoint inhibitor in PBS with 1 %BSA to the respective wells (a particularly suitable range of serial dilutions could comprise 1 µM, 0.5 µM, 0.25 µM, 0.125 µM, 0.06 µM, 0.03 µM, 0.015 µM, 8 nM, 4 nM, 2 nM, 1 nM, 0.3 nM, 0.1 nM, 0.03 nM, 0.01 nM, 0.003 nM and 0.001 nM), incubate for 1 hour at room temperature
6. subsequently, remove supernatant and wash each well with 4 x with 200 µL washing solution
7. subsequently, add 100 µL per well of capture antibody solution in PBS with 1 % BSA) (e.g. goat anti human IgG-HRP, at 50 ng/ml to 5 µg/ml, typically to be determined by titration), incubate 45 min at room temperature
8. subsequently, remove supernatant and wash each well with 6 x with 200 µL washing solution
9. subsequently, add 100 µL per well of TMB substrate
10. upon sufficient completion of the reaction (when a color gradient is visible between the different present immune checkpoint inhibitor dilutions, typically 5-20 minutes after substrate addition), add 100 µL 250 mM sulfuric acid per well (to stop the reaction)
11. subsequently, measure absorption at 450 nm in a suitable plate reader (e.g. Tecan Sunrise)
12. plot data as relative absorption at 450 nm versus present immune checkpoint inhibitor concentration and calculate EC₅₀ values using a suitable curve fit to a suitable pharmacological model (e.g. Emax model) using a suitable software (e.g. Graphpad Prism).

For instance, recombinant immune checkpoint protein can be Human LAG-3 /CD223 Protein (Acro Biosystems, LA3-H5222, Newark, USA), Human PD-1 / PDCD1 protein (Acro Biosystems, PD-1-H52221, Newark, USA), or Human PD-L1 / B7-H1 protein (Acro Biosystems, PD-1-C52H4, Newark, USA). Anti-present immune checkpoint antibody can be, in specific forms of the assay: goat anti-human IgG (H+L) affinity purified antibody (R&D System, G-101-C-ABS, Minneapolis, USA).

Alternatively, the present immune checkpoint inhibitor can be biotinylated using e.g. the Biotin (type B) conjugation Kit (Abcam, ab102867 according to manufacturer’s instructions and detected using a Streptavidin-HRP conjugate, e g. Streptavidin (HRP) (Abcam, ab7403).

Alternative competition format: The above assay procedure can be run in the competition format which is e.g. suitable to determine binding of small molecule present immune checkpoint inhibitors.

In step 5, add serial dilutions of present immune checkpoint inhibitors (a particularly suitable range of serial dilutions could comprise 1 µM, 0.5 µM, 0.25 µM, 0.125 µM, 0.06 µM, 0.03 µM, 0.015 µM, 8 nM, 4 nM, 2 nM, 1 nM, 0.3 nM, 0.1 nM, 0.03 nM, 0.01 nM, 0.003 nM and 0.001 nM) and add recombinant present immune checkpoint ligand (e.g. in serial dilutions in a matrix pattern versus serial dilutions of present immune checkpoint inhibitors to determine suitable concentration of present immune checkpoint ligand, which may be in a similar range as the aforementioned dilutions). In step 7, the capture antibody is specific for the recombinant present immune checkpoint ligand. Suitable IC₅₀ values are 100 nM or lower, particularly 75 nM or lower, more particularly 50 nM or lower.

The ability of immune checkpoint inhibitors which are biologicals to bind their respective targets (e.g. PD-1, PD-L1, PD-L2 or CTLA-4) can be assessed by in vitro/ in vivo and/or cell-based assays either using purified domains of the target proteins or cells using ELISA or flow cytometry methods with a wide array of assays, e.g. the ELISA assay as described herein. Similarly, the ability of the antibodies to block the interaction with their respective ligands or in general can generate a biological response can be evaluated in a similar way (in vitro and/ or cell-based) using designated ligand/ receptor binding systems or biological assays. Exemplary methods for in vitro characterization of immune checkpoint modulators are described in: Cancer Immunol Res. 2014 Sep; 2(9): 846-56 and Cancer Immunol Res. 2015 Sep; 3(9): 1052-62.

Several of the specific present immune checkpoint inhibitors described herein are commercially available and methods of their preparation are available from the related literature.

The present immune checkpoint inhibitors may be small molecules (having a molecular weight of about 600 or lower, particularly 500 or lower, more particularly 400 or lower) or biologicals (as used herein such as antibodies, modified antibodies, antibody fragments and other proteins).

In particular embodiments, the present immune checkpoint inhibitors, more particularly all of the present immune checkpoint inhibitors are antibodies, more particularly human antibodies or humanized antibodies.

The present immune checkpoint inhibitors may be administered in a fixed dose, which means a dose that is equally administered to every patient that does not take into account the respective patient’s body weight, in particular in the case of a biomolecule like an antibody.

The present immune checkpoint inhibitors are to be administered in a dose that is typically used by the physician for the respective present immune checkpoint inhibitor, in particular the dose approved by the respective governmental authorities. In certain embodiments, present immune checkpoint inhibitors that are biologicals (such as antibodies, modified antibodies, antibody fragments and other proteins) may be administered only on day one of a treatment cycle, which may be a particular treatment cycle as described herein. This is due to their long half-life in the patient’s system.

The term antibody in the meaning of the invention comprises all antibodies, antibody fragments, and derivatives thereof that are capable of binding to an antigen, in this case the aforementioned immune respective checkpoints. This encompasses the complete monoclonal antibodies and also the epitope-binding fragments of these antibodies. In this connection, the epitope binding fragments (also referred to herein as antibody fragments or antibody derivatives) comprise all regions of the antibody that are capable of binding to the antigen. Examples of particular antibody fragments in accordance with the invention comprise, but expressly are not limited to, Fab, Fab′, F(ab′)2, Fd, individual chain (single chain) variable fragments (scFv), single-chain antibodies, disulfide-linked variable fragments (sdFv), and fragments that either contain a variable region of the light chain (V_{L}) or a variable region of the heavy chain (V_{H}). Moreover, they include recombinantly prepared antibodies, such as diabodies, and tetrabodies.

Antibody fragments contain the variable regions either alone or in combination with further regions that are selected from the hinge region and the first, second and third regions of the constant region (C_{H}1, C_{H}2, C_{H}3). Also, the term antibody comprises chimeric antibodies in which different regions of the antibody originate from different species, for example, antibodies with a murine variable region combined with a human constant region.

Antibody fragments are optionally linked with each other by a linker. The linker comprises a short (particularly 10 to 20 amino acid residues), flexible peptide sequence that is selected such that the antibody fragment has such a three-dimensional folding of VL and VH that it exhibits the antigen specificity of the complete antibody. Particular linkers are glycine-serine linkers with the structure (GlyₓSer_{y}) with x and y selected from 1 to 10, particularly 3 to 5. Moreover, particular linkers are comprised of a peptide sequence that can increase the protease resistance of the antibody derivatives.

In particular embodiments pembrolizumab is administered in a dose of 2 mg/kg, or in a dose of 200 mg, nivolumab is administered in a dose of 3 mg/kg, or in a dose of 240 mg or in a dose of 480 mg, avelumab is administered in a dose of 10 mg/kg, atezolizumab is administered in a dose of 1200 mg, durvalumab is administered in a dose of 1500 mg. Particularly, pembrolizumab is administered in a dose of 2 mg/kg, more particularly every three weeks, or alternatively in a (fixed) dose of 200 mg, more particularly every three weeks. Particularly, nivolumab is administered in a dose of 3 mg/kg, more particularly every two weeks, or alternatively in a (fixed) dose of 240 mg, more particularly every two weeks, or alternatively in a (fixed) dose of 480 mg, more particularly every four weeks. Particularly, Particularly, avelumab is administered in a dose of 10 mg/kg, more particularly every two weeks. Particularly, atezolizumab is administered in a (fixed) dose of 1200 mg, more particularly every three weeks. Particularly, durvalumab is administered in a (fixed) dose of 1500 mg, more particularly every four weeks.

The treatment cycles as described herein can be repeated one or more times, and typically are repeated as often as necessary, which is typically to be determined by the physician, e.g. based on the disease state (progressive disease, stable disease, tumor regression, etc.), and/or the tolerability of the treatment.

In certain embodiments, the cancer is a solid or hematological tumor;

In certain particular embodiments, the cancer is refractory, non-responding or relapsed to immune checkpoint inhibitor therapy, more particularly therapy with one or more of the present immune checkpoint inhibitors, even more particularly therapy with PD-1 or PD-L1 immune checkpoint inhibitors, wherein even more particularly “refractory” means no stabilization is achieved with immune checkpoint inhibitor therapy, “non-responding” means the best response achieved with immune checkpoint inhibitor therapy is stable disease for 6 months or less followed by disease progression, “relapsed” means temporary response shrinkage followed by disease progression, and wherein disease status including response, progression, stabilization may be determined e.g. according to RECIST or immune related RECIST (irRECIST) criteria version- reference Eisenhauer et al. 2009 Eur J Cancer, 45, 228-247; Nishino M et al., Clin Cancer Res. 2013, Jul 15;19(14):3936-4 3).

In certain particular embodiments, the cancer is an immunologically hot cancer. Immunologically hot means in particular that the tumor is sufficiently infiltrated by T-cells; visible by the immune system; exhibiting tumor antigen presentation, e.g. via MHC I or II. This can be determined e.g. via immune histochemistry, methods of which are well known in the field, such as for example the methods described in Arpita Kabiraj et al., Int J Biol Med Res. 2015; 6(3): 5204-5210 and references therein to the specific methods: Particularly, the cancer is a tumor with an immune cell infiltration corresponding to an immunoscore of 1-4, more particularly 2-4.

In certain embodiments, the subject or patient is refractory or non-responding or relapsed to, immune checkpoint inhibitor therapy, wherein non-responding relates in particular in particular to a response rate that is lower than 20%, more particularly lower than 10%, in particular lower than 10% in prospective clinical studies. In particular, said immune checkpoint inhibitor therapy is adaptive immunity-affecting Checkpoint Inhibitor therapy, more particularly a therapy comprising the administration of a PD-1, PD-L1 or CTLA-4 immune checkpoint inhibitor, wherein said immune checkpoint inhibitor may be administered alone or in combination with other active agents.

In certain embodiments, the cancer shows a PD-L1 expression of less than 1%, in particular compared to average expression in non-cancerous cells of the same cell type.

In other embodiments, the cancer shows a PD-L1 expression of 1% to 5%, in particular compared to average expression in non-cancerous cells of the same cell type.

In other embodiments, the cancer shows a PD-L1 expression of 5% to 50%, in particular compared to average expression in non-cancerous cells of the same cell type.

In other embodiments, the cancer shows a PD-L1 expression of greater than 50%, in particular compared to average expression in non-cancerous cells of the same cell type.

In certain embodiments, the cancer is negative predictive for efficacy to immune checkpoint inhibitor therapy. In other embodiments, the cancer is positive predictive for efficacy to immune checkpoint inhibitor therapy. In particular, said immune checkpoint inhibitor therapy is adaptive immunity-affecting Checkpoint Inhibitor therapy, more particularly a therapy comprising the administration of a PD-1, PD-L1 or CTLA-4 immune checkpoint inhibitor, wherein said immune checkpoint inhibitor may be administered alone or in combination with other active agents.

In certain embodiments, the cancer has a low or medium tumor mutational burden, in particular lower than 20, more particularly lower than 10 mutations per 1 million nucleotide bases. In other embodiments, the cancer has a high tumor mutational burden, in particular higher than 20 mutations per 1 milion nucleotide bases.

Immunologically hot or cold tumors may in certain cases be determined based on certain cellular parameters, such as TGFbeta, PD-L1 and CD8 expression (as described in Mariathasan, S. et al. 2018, Nature. 554:544-548. doi:10.1038/nature25501); mutational load (as described in Yarchoan, M. et al., 2017, N. Engl. J. Med. 377:2500-2501. doi:10.1056/NEJMc1713444); Immunoscore (based on localization, number and type of immune cells), (as described in Galon, J. et al. 2012, J. Transl. Med. 10:1. doi:10.1186/1479-5876-10-1); gut micobiome (degree of heterogeneity and prevalence of certain microbial species) (as described in Routy, B. et al., 2017, Science (80-. ). 3706:eaan3706. doi:10.1126/science.aan3706); immune cell populations and their effects on the prognosis of patients with cancer (as reviewed in Fridman, W.H. et a., 2017, Nat. Rev. Clin. Oncol. 5-8. doi:10.1038/nrclinonc.2017.101), and further factors as reviewed in (Ann Transl Med 2017;5(23):468): baseline microbiome enriched with Faecalibacterium genus and other Firmicutes; neutrophil to lymphocyte ratio; neutrophil number and LDH (lactate dehydrogenase) level; relative eosinophil count and relative lymphocyte count; density of CD8+ T cells and beta-catenin; presence or absence of JAK 2 (Janus kinase 2) and beta-2-microglobulin mutations; TIM-3 (T-cell immunoglobulin mucin-3) level; mutational load; level of tumor tetrapeptide sequences capable of binding to MHC class I molecules (neoantigens); neoantigen intratumor heterogeneity and a amount of clonal neoantigen burden; PD-L1 (programmed death-1 receptor ligand) expression level; PTEN (phosphatase and tensin homolog) expression level; CD8+ T cell expansion; broadness of T cell receptor repertoire (per beta chain) at baseline.

In certain particular embodiments, the cancer is suitable for treatment with one or more of the present immune checkpoint inhibitors, wherein this is particularly a cancer for which a therapy including one or more of the present immune checkpoint inhibitors is approved, i.e. that has received market approval by the regulatory authorities in at least one country.

In certain particular embodiments, the cancer is selected from the group consisting of melanoma (in particular ocular and uveal, but also including skin melanoma), head and neck, renal, Non-small cell lung cancer (NSCLC), microsatellite-instable carcinoma (lynch syndrome, in particular gastroesophageal and colorectal), urothelial carcinoma including bladder cancer, merkel cell carcinoma, hodgkin lymphoma, gastric, gastrointestinal cancers (microsatellite stable and instable) including colorectal cancer (CRC), hepatocellular carcinoma (HCC), renal cell carcinoma (RCC), nasopharyngeal, basal cell carcinoma, cervical cancer, anogenital cancers, Kaposi sarcoma, adult T-cell leukemia, primary effusion lymphoma, and Castlemann’s disease.

In certain particular embodiments, the cancer is selected from the group consisting of breast cancer, in particular triple-negative breast cancer, oesophageal cancer, non-hodgkin lymphoma, small cell lung cancer (SCLC), sarkoma, mesothelioma, glioblastoma, microsatellite stable cancer (in particular gastroesophageal and colorectal), pancreas cancer, prostate cancer, cutaneous T-cell lymphoma (CTCL), and squamous cell carcinoma.

The number of immune cells and/or its ratio versus the total cell number in a tumor in the context of the present invention is determinable by standard methods known to the skilled person and in particular embodiments determinable in a formalin-fixed paraffin-embedded tumor sample obtainable from the patient by
1) cutting a 5-10 µM slice of said sample,
2) fixing the slices in 4% PF (paraffin),
3) rinsing twice in PBS for 2 minutes,
4) adding commercially available serum (5% in PBS) and incubating for 20 minutes,
5) adding a primary commercially available antibody against CD3+ or CD8+ and incubating for 60 min (dilution of 5 µg/ml in PBS),
6) rinsing twice in PBS for 2 minutes,
7) adding a secondary biotinylated antibody (binding to the constant region of the primary antibody) and incubating for 30 min,
8) rinsing twice in PBS for 2 minutes,
9) add streptavidin-peroxidase (e.g. Jackson Immunoresearch), incubate for 30 min,
10) rinsing twice in PBS for 2 minutes,
11) add developer (e.g. AEC Substrate Chromogen Ready-to-Use, Dako # K3464), particularly until sufficiently stained (typically observe development under microscope, typically for 5 min)
12) rinse with water
13) counterstain with commercially available HTX solution
14) mount in water-based mounting media (e.g. DAKO)
15) determine CD3+ or CD8+ cell number
16) optionally determine cell number ratio by dividing CD3+ or CD8+ cell number by total cell number in tumor volume (e.g. based on typical cell numbers in said specific cancer type);

Or by the following assay

For each tumor sample, stain 2 slides are using an automated immunohistochemistry staining instrument (BenchMark XT, Ventana): one with CD3 and one with CD8 ready-to-use monoclonal antibodies (HalioDx).

Perform staining with ultraView Universal DAB Detection Kit (Ventana), followed by counterstaining (Bluing Reagent, Ventana).

Wash stained slides, dehydrate, mount and coverslip.

Obtain digital images of stained slides using a whole slide scanner (Nanozoomer XR, Hamamatsu), and analyze by a software program (Immunoscore ®Analyzer, HalioDx) or count to determine cell numbers and optionally determine ratio as above.

(Optional: One separate control slide with 3 external controls -1 negative tissue (placenta) and 2 positive (1 tissue: tonsil and a cell line pellet) - is processed identically in each IHC run, and allows monitoring of the staining and scanning steps.)

In certain embodiments the patient having said cancer has received at least one prior systemic treatment against said cancer, e.g. at least one prior systemic chemotherapeutic treatment against said cancer, e.g. at least one prior systemic treatment comprising the administration of one or more of the present immune checkpoint inhibitors.

In certain embodiments of the present invention, said prior systemic chemotherapeutic treatment is a treatment of administrating one or more chemotherapeutic agents systemically, such chemotherapeutic agent may be used alone or in combination with further agents.

In certain embodiments of the present invention, said patient having said cancer has received at least one prior systemic treatment comprising the administration of one or more of the present immune checkpoint inhibitors against said cancer and said patient was a non-responder or said cancer was refractory or relapsed to said at least one prior systemic treatment.

In the present invention, the treatment may be the treatment of a subject suffering from cancer, in particular a human subject suffering from cancer, in particular the specific cancer types described herein. Said subject may also be addressed as a patient, as used herein in certain contexts.

The HDAC inhibitor is meant to be inclusive of the respective salts, solvates and hydrates.

In the present invention, the HDAC inhibitor and all of the present immune checkpoint inhibitors are typically to be administered in therapeutically effective amounts.

In certain particular embodiments the HDAC inhibitor is Class I HDAC specific. In particular, the HDAC inhibitor is specific for one or more class I HDAC enzymes (HDAC 1, HDAC 2 and/or HDAC3), wherein specific particularly means that the Ki for said one or more class I HDAC is at least 2-fold, more particularly at least 5-fold, even more particularly at least 10-fold, even more particularly at least 50-fold lower than the Ki for an HDAC enzyme of any other class (i.e. in particular an enzyme of the group consisting of HDAC classes IIA IIB, III and IV). Assays by which HDAC inhibition is determinable are e.g. the assays described in WO 2005/087724 A2 and WO 2006/097474 A1.

The HDAC inhibitor is in a first aspect (aspect A) a compound of formula I

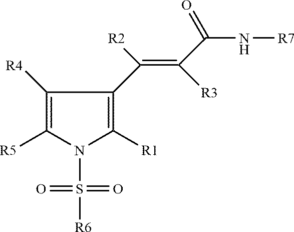

in which
R1, R4 and R5 are independently hydrogen, 1-4C-alkyl, halogen, or 1-4C-alkoxy,
R2 and R3 are independently hydrogen or 1-4C-alkyl,
R6 is -T1 -Q1, in which T1 is a bond or 1-4C-alkylene, either
Q1 is substituted by R61 and/or R62, and is Aa1, Hh1, Ha1, Ha2, Ha3, Ha4 or Ah1, or
Q1 is unsubstituted, and is Ha2, Ha3 or Ha4,
in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, -U-T3-N(R613)R614, -T4-Het3, or -V-T5-Het4, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl, 1-4C-alkylcarbonyl, or 1-4C-alkylsulphonyl
R614 is hydrogen or 1-4C-alkyl,
or R613 and R614 together and with inclusion of the nitrogen atom, to which they are bonded, form a heterocyclic ring Het2, in which
Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
T4 is a bond or 1-4C-alkylene,
Het3 is 1N-(1-4C-alkyl)-piperidinyl or 1N-(1-4C-alkyl)-pyrrolidinyl,
V is —O— (oxygen) or —C(O)NH—,
T5 is a bond or 1-4C-alkylene,
Het4 is 1N-(1-4C-alkyl)-piperidinyl or 1N-(1-4C-alkyl)-pyrrolidinyl,
R62 is 1-4C-alkyl, 1-4C-alkoxy or halogen,
Aa1 is a bisaryl radical made up of two aryl groups,
   which are selected independently from a group consisting of phenyl and naphthyl, and
   which are linked together via a single bond,
Hh1 is a bisheteroaryl radical made up of two heteroaryl groups, which are selected independently from a group consisting of monocyclic 5- or 6-membered heteroaryl radicals comprising one or two heteroatoms, each of which is selected from the group consisting of nitrogen, oxygen and sulfur, and which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group,
R7 is hydroxyl, or Cyc1, in which Cyc1 is a ring system of formula Ia

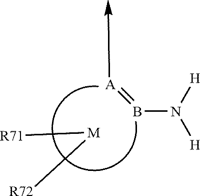

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring, Har2 is a monocyclic 5- or 6-membered unsaturated heteroaromatic ring comprising one to three heteroatoms, each of which is selected from the group consisting of nitrogen, oxygen and sulfur,
and the salts of these compounds.

The HDAC inhibitor is in a second aspect (aspect B), which is an embodiment of aspect A, a compound of formula I, in which
R1, R4 and R5 are independently hydrogen, 1-4C-alkyl, halogen, or 1-4C-alkoxy,
R2 and R3 are independently hydrogen or 1-4C-alkyl,
R6 is -T1 -Q1, in which T1 is a bond or 1-4C-alkylene, either
Q1 is substituted by R61 and/or R62, and is Aa1, Hh1, Ha1, Ha2, Ha3 or Ah1,
or Q1 is unsubstituted, and is Ha2 or Ha3,
in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, or -U-T3-N(R613)R614, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl or 1-4C-alkoxy-2-4C-alkyl,
R612 is hydrogen or 1-4C-alkyl,
or R611 and R612 together and with inclusion of the nitrogen atom, to which they are bonded, form a heterocyclic ring Het1, in which
Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is -O- (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl,
R614 is hydrogen or 1-4C-alkyl,
or R613 and R614 together and with inclusion of the nitrogen atom, to which they are bonded, form a heterocyclic ring Het2, in which Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
R62 is 1-4C-alkyl, 1-4C-alkoxy or halogen,
Aa1 is a bisaryl radical made up of two aryl groups,
   which are selected independently from a group consisting of phenyl and naphthyl, and
   which are linked together via a single bond,
Hh1 is a bisheteroaryl radical made up of two heteroaryl groups, which are selected independently from a group consisting of monocyclic 5- or 6-membered heteroaryl radicals comprising one or two heteroatoms, each of which is selected from the group consisting of nitrogen, oxygen and sulfur, and which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
R7 is hydroxyl, or Cyc1, in which Cyc1 is a ring system of formula Ia

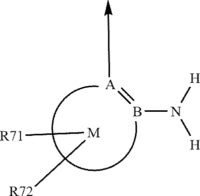

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which
Ar2 is a benzene ring,
Har2 is a monocyclic 5- or 6-membered unsaturated heteroaromatic ring comprising one to three heteroatoms, each of which is selected from the group consisting of nitrogen, oxygen and sulfur,
and the salts of these compounds.

The HDAC inhibitor is in a third aspect (aspect C), which is also an embodiment of aspect A, a compound of formula I, in which
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond, or 1-4C-alkylene, either
Q1 is substituted by R61 and/or R62, and is Aa1, Hh1, Ha1, Ha2, Ha3 or Ah1,
or Q1 is unsubstituted, and is Ha2 or Ha3,
in which
R61 is 1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, or -T2-N(R611)R612, in which
T2 is a bond or 1-4C-alkylene,
R611 and R612 are indenpendently hydrogen or 1-4C-alkyl,
or R611 and R612 together and with inclusion of the nitrogen atom, to which they are bonded, form a heterocyclic ring Het1, in which
Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
R62 is 1-4C-alkyl, 1-4C-alkoxy or halogen,
Aa1 is a bisaryl radical made up of two aryl groups,
   which are selected independently from a group consisting of phenyl and naphthyl, and
   which are linked together via a single bond,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an aryl-heteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
R7 is hydroxyl, or Cyc1, in which Cyc1 is a ring system of formula Ia

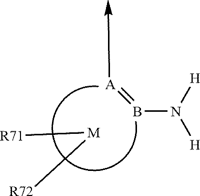

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring,
Har2 is a monocyclic 5- or 6-membered unsaturated heteroaromatic ring comprising one to three heteroatoms, each of which is selected from the group consisting of nitrogen, oxygen and sulfur,
and the salts of these compounds.

1-4C-Alkyl represents a straight-chain or branched alkyl radical having 1 to 4 carbon atoms. Examples which may be mentioned are the butyl, isobutyl, sec-butyl, tert-butyl, propyl, isopropyl and particularly the ethyl and methyl radicals.

2-4C-Alkyl represents a straight-chain or branched alkyl radical having 2 to 4 carbon atoms. Examples which may be mentioned are the butyl, isobutyl, sec-butyl, tert-butyl, propyl, isopropyl and particularly the ethyl radicals.

3-7C-Cycloalkyl stands for cyclopropyl, cyclobutyl, cyclopentyl, cyclohexyl and cycloheptyl, of which cyclopropyl, cyclobutyl and cyclopentyl are particular examples.

3-7C-Cycloalkylmethyl stands for a methyl radical, which is substituted by one of the abovementioned 3-7C-cycloalkyl radicals. Particular examples which may be mentioned are the cyclopropylmethyl, the cyclobutylmethyl and the cyclopentylmethyl radicals.

1-4C-Alkylene is a branched or, particularly, straight chain alkylene radical having 1 to 4 carbon atoms. Examples which may be mentioned are the methylene (—CH₂—), ethylene (dimethylene) (—CH₂—CH₂—), trimethylene (—CH₂—CH₂—CH₂—) and the tetramethylene (—CH₂—CH₂—CH₂—CH₂—) radical.

2-4C-Alkylene is a branched or, particularly, straight chain alkylene radical having 2 to 4 carbon atoms. Examples which may be mentioned are the ethylene (dimethylene) (—CH₂—CH₂—), trimethylene (—CH₂—CH₂—CH₂—) and the tetramethylene (—CH₂—CH₂—CH₂—CH₂—) radical.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and particularly the ethoxy and methoxy radicals.

1-4C-Alkoxy-1-4C-alkyl stands for one of the abovementioned 1-4C-alkyl radicals, which is substituted by one of the abovementioned 1-4C-alkoxy radicals. Examples which may be mentioned are the methoxymethyl, 2-methoxyethyl, 3-methoxypropyl and the 2-ethoxyethyl radical.

1-4C-Alkoxy-2-4C-alkyl stands for one of the abovementioned 2-4C-alkyl radicals, which is substituted by one of the abovementioned 1-4C-alkoxy radicals. Examples which may be mentioned are the 2-methoxyethyl, 3-methoxypropyl and the 2-ethoxyethyl radical.

Hydroxy4C-alkyl stands for one of the abovementioned 1-4C-alkyl radicals which is substituted by hydroxyl. Examples which may be mentioned are the hydroxymethyl radical, the 2-hydroxyethyl radical or the 3-hydroxypropyl radical.

Hydroxy4C-alkyl stands for one of the abovementioned 2-4C-alkyl radicals which is substituted by hydroxyl. Examples which may be mentioned are the 2-hydroxyethyl radical or the 3-hydroxypropyl radical.

Phenyl4C-alkyl represents one of the abovementioned 1-4C-alkyl radicals, which is substituted by a phenyl radical. Examples which may be mentioned are the benzyl and phenethyl radicals.

Mono- or Di-1-4C-alkylamino radicals contain in addition to the nitrogen atom, one or two of the abovementioned 1-4C-alkyl radicals. Particular examples red are the di-1-4C-alkylamino radicals, especially the dimethylamino, the diethylamino and the diisopropylamino radical.

Mono- or Di-1-4C-alkylaminocarbonyl radicals contain in addition to the carbonyl group one of the abovementioned mono- or di-1-4C-alkylamino radicals. Examples which may be mentioned are the N-methyl- the N,N-dimethyl-, the N-ethyl-, the N-propyl-, the N,N-diethyl-and the N-isopropylaminocarbonyl radical, of which the N,N-dimethylaminocarbonyl radical is a particular example.

Mono- or Di-1-4C-alkylaminosulphonyl stands for a sulphonyl group to which one of the abovementioned mono- or di-1-4C-alkylamino radicals is bonded. Examples which may be mentioned are the methylaminosulphonyl, the dimethylaminosulphonyl and the ethylaminosulphonyl radical, of which the N,N-dimethylaminosulphonyl (dimethylsulphamoyl) radical [(CH₃)₂NS(O)₂—] is a particular example.

An 1-4C-Alkylcarbonylamino radical is, for example, the propionylamino (C₂H₅C(O)NH—) and the acetylamino (acetamido) radical (CH₃C(O)NH—).

An 1-4C-Alkylsulphonylamino radical is, for example, the ethanesulphonylamino (ethylsulphonylamino) (C₂H₅S(O)₂NH—) and the methanesulphonylamino (methylsulphonylamino) radical (CH₃S(O)₂NH—).

1-4C-Alkylsulfonyl is a sulfonyl group to which one of the abovementioned 1-4C-alkyl radicals is bonded. An example is the methanesulphonyl (methylsulphonyl) radical (CH₃SO₂—).

1-4C-Alkylcarbonyl is a carbonyl group to which one of the abovementioned 1-4C-alkyl radicals is bonded. An example is the acetyl radical (CH₃CO—).

Tolyl alone or as part of another group includes o-tolyl, m-tolyl and p-tolyl.

Halogen within the meaning of the invention is bromine or, in particular, chlorine or fluorine.

Aa1 is a bisaryl radical made up of two aryl groups, which are selected independently from a group consisting of phenyl and naphthyl, and which are linked together via a single bond.

Aa1 may include, without being restricted thereto, the biphenyl radical, e.g. the 1,1′-biphenyl-4-yl or 1,1′-biphenyl-3-yl radical.

As non-limiting examples of R61-substituted derivatives of Aa1 may be mentioned the following radicals:

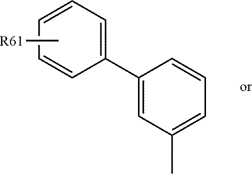

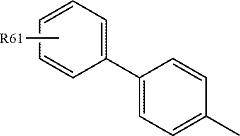

in which the substituent R61 can be attached in the ortho, or, in particular, meta or para position with respect to the binding position in which the benzene ring is bonded to the phenyl radical, such as e.g. 2′-(R61)-1,1′-biphenyl-3-yl, 2′-(R61)-1,1′-biphenyl-4-yl, or, in particular, 3′-(R61)-1,1′-biphenyl-3-yl or 3′-(R61)-1,1′-biphenyl-4-yl, or, yet in particular, 4′-(R61)-1,1′-biphenyl-3-yl or 4′-(R61)-1,1′-biphenyl-4-yl.

As exemplary R61-substituted Aa1 radicals may be more detailed mentioned, for example, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is -T2-N(R611)R612, in which
T2 is methylene, dimethylene or trimethylene, and
R611 and R612 together and with inclusion of the nitrogen atom, to which they are bonded, form a morpholino or 4N-methyl-piperazino, or a piperidino or pyrrolidino radical; such as, for example, any selected from
3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl and 4′-(4-methyl-piperazin-1-ylmethyl)-biphenyl-3-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is -T2-N(R611)R612, in which T2 is methylene, dimethylene or trimethylene, and
R611 and R612 are both methyl;
such as, for example, any selected from
2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl and 3′-dimethylaminomethyl-biphenyl-3-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is -T2-N(R611)R612, in which
T2 is methylene, dimethylene or trimethylene, and
R611 is hydrogen, cyclopropyl, cyclopentyl, 2-methoxyethyl, acetyl or methylsulfonyl,
R612 is hydrogen;
for example, either
R611 is cyclopropyl or 2-methoxyethyl, and
R612 is hydrogen, such as, for example, any selected from 4′-(2-methoxy-ethylamino)methyl-biphenyl-3-yl and 4′-cyclopropylaminomethyl-biphenyl-3-yl, or
R611 is hydrogen, cyclopentyl, acetyl or methylsulfonyl, and
R612 is hydrogen,
such as, for example, any selected from 4′-aminomethyl-biphenyl-3-yl, 4′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl and 4′-cyclopentylaminomethyl-biphenyl-4-yl.

Yet as exemplary R61-substituted Aa1 radicals may be more detailed mentioned, for example, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is -O-T3-N(R613)R614, in which T3 is dimethylene or trimethylene, and
R613 and R614 together and with inclusion of the nitrogen atom, to which they are bonded, form a morpholino, pyrrolidino or 4N-methyl-piperazino, or a piperidino radical;
such as, for example, any selected from 4′-(2-morpholin-4-yl-ethoxy)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, 4′-(2-pyrrolidin-1-yl-ethoxy]-biphenyl-3-yl, 3′-(3-pyrrolidin-1-yl-propoxy]-biphenyl-4-yl, 4′-(3-pyrrolidin-1-yl-propoxy]-biphenyl-4-yl, 3′-(2-pyrrolidin-1-yl-ethoxy]-biphenyl-4-yl, 4′-(3-morpholin-4-yl-propoxy)-biphenyl-4-yl, 3′-(3-morpholin-4-yl-propoxy)-biphenyl-4-yl, 3′-(2-morpholin-4-yl-ethoxy)-biphenyl-4-yl, 4′-(2-morpholin-4-yl-ethoxy)-biphenyl-4-yl, 4′-[2-(4-methyl-piperazin-1-yl)-ethoxy]-biphenyl-4-yl, 4′-[3-(4-methyl-piperazin-1-yl)-propoxy]-biphenyl-4-yl and 3′-[3-(4-methyl-piperazin-1-yl)-propoxy]-biphenyl-4-yl.

Yet as exemplary R61-substituted Aa1 radicals may be more detailed mentioned, for example, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is -O-T5-Het4, in which T5 is a bond, methylene, dimethylene or trimethylene, and
Het4 is 1-methyl-piperidin-4-yl;
such as e.g. 4′-(2-(1 -methyl-piperidin-4-yl)-ethoxy)-biphenyl-4-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is methylsulphonylamino, N,N-dimethylaminosulphonyl, acetamido, hydroxymethyl, amino, dimethylamino, morpholino, hydroxyl, trifluoromethyl or methoxy; for example, either
R61 is methylsulphonylamino, N,N-dimethylaminosulphonyl, acetamido or hydroxymethyl, such as, for example, any selected from 2′-methylsulphonylamino-biphenyl-4-yl, 3′-methylsulphonylamino-biphenyl-4-yl, 4′-methylsulphonylamino-biphenyl-4-yl, 4′-methylsulphonylamino-biphenyl-3-yl, 4′-dimethylsulphamoyl-biphenyl-4-yl, 3′-acetamido-biphenyl-4-yl, 4′-acetamido-biphenyl-4-yl and 3′-hydroxymethyl-biphenyl-4-yl, or
R61 is amino, dimethylamino, morpholino, hydroxyl, trifluoromethyl or methoxy, such as, for example, any selected from 3′-amino-biphenyl-4-yl, 4′-morpholin-4-yl-biphenyl-4-yl, 4′-hydroxy-biphenyl-4-yl, 3′-trifluoromethyl-biphenyl-4-yl, 3′-dimethylamino-biphenyl-4-yl and 4′-methoxy-biphenyl-4-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is -C(O)-N(H)-T3-N(R613)R614, in which T3 is dimethylene or trimethylene, and
R613 and R614 are both methyl;
such as, for example, any selected from 3′-[(2-dimethylamino-ethylamino)-carbonyl]-biphenyl-4-yl, 4′-[(2-dimethylamino-ethylamino)-carbonyl]-biphenyl-4-yl and 4′-[(2-dimethylamino-ethylamino)-carbonyl]-biphenyl-3-yl.

An example of R61-substituted Aa1 radicals may be 3′-(R61)-1,1′-biphenyl-3-yl, in which R61 is any one selected from the group G_{Aa1} consisting of 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl.

Another example of R61-substituted Aa1 radicals may be 3′-(R61)-1,1′-biphenyl-4-yl, in which R61 is any one selected from the group G_{Aa1} given above.

Another example of R61-substituted Aa1 radicals may be 4′-(R61)-1,1′-biphenyl-3-yl, in which R61 is any one selected from the group G_{Aa1} given above.

Another example of R61-substituted Aa1 radicals may be 4′-(R61)-1,1′-biphenyl-4-yl, in which R61 is any one selected from the group G_{Aa1} given above.

Specifically, as an exemplary R61-substituted Aa1 radical may be explicitely mentioned, for example, any one selected from 3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl, 4′-(4-methyl-piperazin-1-ylmethyl)-biphenyl-3-yl, 4′-(2-morpholin-4-yl-ethoxy)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, 4′-(2-pyrrolidin-1-yl-ethoxy]-biphenyl-3-yl, 3′-(3-pyrrolidin-1-yl-propoxy]-biphenyl-4-yl, 4′-(3-pyrrolidin-1-yl-propoxy]-biphenyl-4-yl, 3′-(2-pyrrolidin-1-yl-ethoxy]-biphenyl-4-yl, 4′-(3-morpholin-4-yl-propoxy)-biphenyl-4-yl, 3′-(3-morpholin-4-yl-propoxy)-biphenyl-4-yl, 3′-(2-morpholin-4-yl-ethoxy)-biphenyl-4-yl, 4′-(2-morpholin-4-yl-ethoxy)-biphenyl-4-yl, 4′-[2-(4-methyl-piperazin-1-yl)-ethoxy]-biphenyl-4-yl, 4′-[3-(4-methyl-piperazin-1-yl)-propoxy]-biphenyl-4-yl, 3′-[3-(4-methyl-piperazin-1 -yl)-propoxy]-biphenyl-4-yl, 4′-(2-(1-methyl-piperidin-4-yl)-ethoxy)-biphenyl-4-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl, 3′-dimethylaminomethyl-biphenyl-3-yl, 3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 2′-methylsulphonylamino-biphenyl-4-yl, 3′-methylsulphonylamino-biphenyl-4-yl, 4′-methylsulphonylamino-biphenyl-4-yl, 4′-methylsulphonylamino-biphenyl-3-yl, 4′-dimethylsulphamoyl-biphenyl-4-yl, 3′-acetamido-biphenyl-4-yl, 4′-acetamido-biphenyl-4-yl, 3′-amino-biphenyl-4-yl, 4′-morpholin-4-yl-biphenyl-4-yl, 4′-hydroxy-biphenyl-4-yl, 3′-trifluoromethyl-biphenyl-4-yl and 4′-methoxy-biphenyl-4-yl, 4′-(2-methoxy-ethylamino)methyl-biphenyl-3-yl, 4′-aminomethyl-biphenyl-3-yl, 4′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl, 4′-cyclopentylaminomethyl-biphenyl-4-yl, 4′-cyclopropylaminomethyl-biphenyl-3-yl, and 3′-hydroxymethyl-biphenyl-4-yl.

More specifically, as an exemplary R61-substituted Aa1 radical may be more explicitely mentioned, for example, any one selected from 4′-(2-morpholin-4-yl-ethyl)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, and 4′-dimethylaminomethyl-biphenyl-4-yl.

Hh1 is a bisheteroaryl radical made up of two heteroaryl groups, which are selected independently from a group consisting of monocyclic 5- or 6-membered heteroaryl radicals comprising one or two heteroatoms, each of which is selected from the group consisting of nitrogen, oxygen and sulfur, and which are linked together via a single bond.

Hh1 may include, without being restricted thereto, the bithiophenyl e.g. thiophen-3-yl-thiophenyl or thiophen-2-yl-thiophenyl, bipyridyl, pyrazolyl-pyridinyl e.g. pyrazol-1-yl-pyridinyl or pyrazol-4-yl-pyridinyl like 6-(pyrazol-4-yl)-pyridin-3-yl, imidazolyl-pyridinyl e.g. imidazol-1-yl-pyridinyl, pyrazolyl-thiophenyl e.g. pyrazol-4-yl-thiophenyl like 5-(pyrazol-4-yl)-thiophen-2-yl, or pyridinyl-thiophenyl radical e.g. pyridin-2-yl-thiophenyl, pyridin-3-yl-thiophenyl or pyridin-4-yl-thiophenyl like 5-(pyridin-2-yl)-thiophen-2-yl or 5-(pyridin-4-yl)-thiophen-2-yl, or the thiazolyl-thiophenyl e.g. thiazol-4-yl-thiophenyl like 5-(thiazol-4-yl)-thiophen-2-yl, or thiazolyl-pyridinyl radical like 6-(thiazol-4-yl)-pyridin-3-yl.

In a special detail, exemplary Hh1 radicals may include pyridinyl-thiophenyl, e.g. 5-(pyridin-4-yl)-thiophen-2-yl. In another special detail, exemplary Hh1 radicals may include pyrazolyl-thiophenyl, e.g. 5-(pyrazol-4-yl)-thiophen-2-yl. In another special detail, exemplary Hh1 radicals may include bipyridyl, e.g. 2,4′-bipyridyl-5-yl. In another special detail, exemplary Hh1 radicals may include thiazolyl-thiophenyl, e.g. 5-(thiazol-4-yl)-thiophen-2-yl. In another special detail, exemplary Hh1 radicals may include pyrazolyl-pyridinyl, e.g. 6-(pyrazol-4-yl)-pyridin-3-yl. In another special detail, exemplary Hh1 radicals may include thiazolyl-pyridinyl, e.g. 6-(thiazol-4-yl)-pyridin-3-yl.

As non-limiting example of R61-substituted derivatives of Hh1 may be mentioned [1N-(1-4C-alkyl)-pyrazolyl]-thiophenyl, such as e.g. [1N-(1-4C-alkyl)-pyrazol-4-yl]-thiophenyl, like 5-[1N-(1-2C-alkyl)-pyrazol-4-yl]-thiophen-2-yl, e.g. 5-(1N-methyl-pyrazol-4-yl)-thiophen-2-yl.

Yet as non-limiting example of R61-substituted derivatives of Hh1 may be mentioned [1N-(1-4C-alkyl)-pyrazolyl]-pyridinyl, such as e.g. [1N-(1-4C-alkyl)-pyrazol-4-yl]-pyridinyl or 6-[1N-(1-4C-alkyl)-pyrazolyl]-pyridin-3-yl, like 6-[1N-(1-2C-alkyl)-pyrazol-4-yl]-pyridin-3-yl, e.g. 6-(1N-methyl-pyrazol-4-yl)-pyridin-3-yl.

Yet as non-limiting example of R61-substituted derivatives of Hh1 may be mentioned [(R61)-pyridinyl]-thiophenyl, such as e.g. the following radicals:

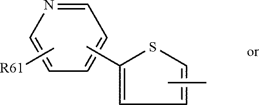

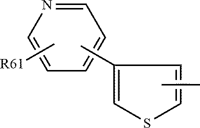

in which the substituent R61 can be attached in the ortho, or, in particular, meta or para position with respect to the binding position in which the pyridinyl ring is bonded to the thiophenyl radical, such as e.g. [2-(R61)-pyridin-4-yl]-thiophenyl or [6-(R61)-pyridin-3-yl]-thiophenyl, like 5-[2-(R61)-pyridin-4-yl]-thiophen-2-yl or 5-[6-(R61)-pyridin-3-yl]-thiophen-2-yl.

Yet as non-limiting example of R61-substituted derivatives of Hh1 may be mentioned [(R61)-thiazolyl]-thiophenyl, such as e.g. the following radicals:

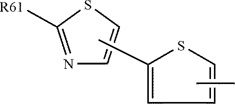

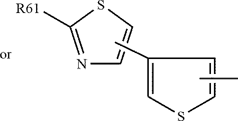

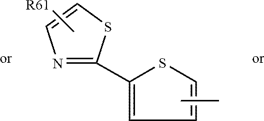

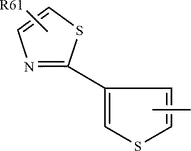

such as e.g. [2-(R61)-thiazol-4-yl]-thiophenyl, like 5-[2-(R61)-thiazol-4-yl]-thiophen-2-yl.

Yet as non-limiting example of R61-substituted derivatives of Hh1 may be mentioned [(R61)-pyridinyl]-pyridinyl, such as e.g. the following radicals:

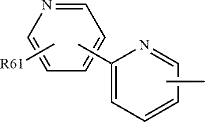

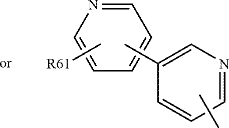

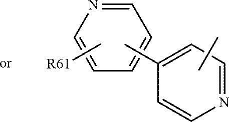

in which the substituent R61 can be attached in the ortho, or, in particular, meta or para position with respect to the binding position in which the terminal pyridinyl ring is bonded to the other pyridinyl radical, such as e.g. [2-(R61)-pyridin-4-yl]-pyridinyl or [6-(R61)-pyridin-3-yl]-pyridinyl or 6-[(R61)-pyridinyl]-pyridin-3-yl, like 6-[2-(R61)-pyridin-4-yl]-pyridin-3-yl [i.e. 2′-(R61)-2,4′-bipyridyl-5-yl] or 6-[6-(R61)-pyridin-3-yl]-pyridin-3-yl [i.e. 6′-(R61)-2,3′-bipyridyl-5-yl].

As exemplary R61-substituted Hh1 radicals may be more detailed mentioned, for example, 5-[2-(R61)-pyridin-4-yl]-thiophen-2-yl or 5-[6-(R61)-pyridin-3-yl]-thiophen-2-yl, in which
R61 is -T2-N(R611)R612, in which T2 is a bond, and
R611 and R612 are both hydrogen, or
R611 and R612 together and with inclusion of the nitrogen atom, to which they are bonded, form a morpholino or 4N-methyl-piperazino, or a piperidino or pyrrolidino radical;
such as e.g. 5-[2-(4-methyl-piperazin-1-yl)-pyridin-4-yl]-thiophen-2-yl.

Yet as exemplary R61-substituted Hh1 radicals may be more detailed mentioned, for example, 2′-(R61)-2,4′-bipyridyl-5-yl or 6′-(R61)-2,3′-bipyridyl-5-yl, in which
R61 is -T2-N(R611)R612, in which T2 is a bond, and
R611 and R612 are both hydrogen, or
R611 and R612 together and with inclusion of the nitrogen atom, to which they are bonded, form a morpholino, 4N-methyl-piperazino, piperidino or pyrrolidino radical;
such as e.g. 2′-(4-methyl-piperazin-1-yl)-2,4′-bipyridyl-5-yl.

Specifically, as an exemplary R61-substituted Hh1 radical may be explicitely mentioned, for example, any one selected from 5-[2-(4-methyl-piperazin-1-yl)-pyridin-4-yl]-thiophen-2-yl, 5-(1N-methyl-pyrazol-4-yl)-thiophen-2-yl, 2′-(4-methyl-piperazin-1-yl)-2,4′-bipyridyl-5-yl, 5-(2-methyl-thiazol-4-yl)-thiophen-2-yl, and 6-(1N-methyl-pyrazol-4-yl)-pyridin-3-yl.

More specifically, as an exemplary R61-substituted Hh1 radical may be more explicitely mentioned, for example, 5-[2-(4-methyl-piperazin-1-yl)-pyridin-4-yl]-thiophen-2-yl.

Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group.

Ah1 may include, without being restricted thereto, the phenyl-thiophenyl e.g. 5-phenyl-thiophen-2-yl, or the phenyl-pyridyl e.g. 6-phenyl-pyridin-3-yl, radical.

In a special detail, exemplary Ah1 radicals may include phenyl-thiophenyl, e.g. 5-(phenyl)-thiophen-2-yl.

Yet in a special detail, exemplary Ah1 radicals may include phenyl-pyridinyl, e.g. 6-(phenyl)-pyridin-3-yl.

As non-limiting example of R61-substituted derivatives of Ah1 may be mentioned [(R61)-phenyl]-thiophenyl, such as e.g. the following radicals:

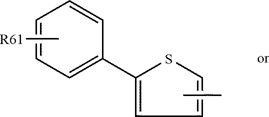

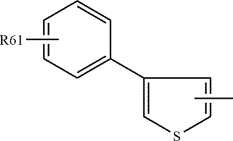

in which the substituent R61 can be attached in the ortho, or, in particular, meta or para position with respect to the binding position in which the phenyl ring is bonded to the thiophenyl radical, such as e.g. [3-(R61)-phenyl]-thiophenyl or [4-(R61)-phenyl]-thiophenyl, like 5-[3-(R61)-phenyl]-thiophen-2-yl or 5-[4-(R61)-phenyl]-thiophen-2-yl.

Yet as non-limiting example of R61-substituted derivatives of Ah1 may be mentioned [(R61)-phenyl]-pyridinyl, such as e.g. the following radicals:

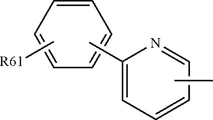

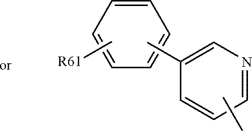

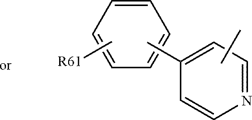

in which the substituent R61 can be attached in the ortho, or, in particular, meta or para position with respect to the binding position in which the phenyl ring is bonded to the pyridinyl radical, such as e.g. [3-(R61)-phenyl]-pyridinyl or [4-(R61)-phenyl]-pyridinyl or 6-[(R61)-phenyl]-pyridin-3-yl, like 6-[3-(R61)-phenyl]-pyridin-3-yl or 6-[4-(R61)-phenyl]-pyridin-3-yl.

As exemplary R61-substituted Ah1 radicals may be more detailed mentioned, for example, 5-[3-(R61)-phenyl]-thiophen-2-yl or 5-[4-(R61)-phenyl]-thiophen-2-yl, in which
R61 is -T2-N(R611)R612, in which T2 is methylene, dimethylene or trimethylene, and
R611 and R612 together and with inclusion of the nitrogen atom, to which they are bonded, form a morpholino or 4N-methyl-piperazino, or a piperidino or pyrrolidino radical;
such as, for example, any selected from 5-[4-(2-morpholin-4-yl-ethyl)-phenyl]-thiophen-2-yl, 5-[4-(morpholin-4-yl-methyl)-phenyl]-thiophen-2-yl and 5-[3-(morpholin-4-yl-methyl)-phenyl]-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which
R61 is -T2-N(R611)R612, in which T2 is methylene, dimethylene or trimethylene, and
R611 and R612 are both methyl;
such as, for example, any selected from 5-(4-dimethylaminomethyl-phenyl)-thiophen-2-yl and 5-(3-dimethylaminomethyl-phenyl)-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which
R61 is -T2-N(R611)R612, in which T2 is methylene, dimethylene or trimethylene, and
R611 is hydrogen, cyclopropyl, cyclopentyl, 2-methoxyethyl, acetyl or methylsulfonyl,
R612 is hydrogen;
such as, for example, any selected from 5-(3-aminomethyl-phenyl)-thiophen-2-yl, 5-[3-(acetylamino)-methyl-phenyl]-thiophen-2-yl and 5-[3-(methylsulphonylamino)-methyl-phenyl]-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which
R61 is methylsulphonylamino, N,N-dimethylaminosulphonyl, acetamido, hydroxymethyl, amino, dimethylamino, morpholino, hydroxyl, trifluoromethyl or methoxy;
such as e.g. 5-(4-dimethylsulphamoyl-phenyl)-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which
R61 is -O-T3-N(R613)R614, in which T3 is dimethylene or trimethylene, and
R613 and R614 together and with inclusion of the nitrogen atom, to which they are bonded, form a morpholino, pyrrolidino or 4N-methyl-piperazino, or a piperidino radical;
such as, for example, any selected from 5-[4-(2-morpholin-4-yl-ethoxy)-phenyl]-thiophen-2-yl, 5-[4-(3-morpholin-4-yl-propoxy)-phenyl]-thiophen-2-yl, 5-{4-[2-(4-methyl-piperazin-1-yl)-ethoxy]-phenyl}-thiophen-2-yl and 5-[4-(2-pyrrolidin-1-yl-ethoxy)-phenyl]-thiophen-2-yl.

Yet as exemplary R61-substituted Ah1 radicals may be more detailed mentioned, for example, 6-[3-(R61)-phenyl]-pyridin-3-yl or 6-[4-(R61)-phenyl]-pyridin-3-yl, in which
R61 is -T2-N(R611)R612, in which T2 is methylene, dimethylene or trimethylene, and
R611 and R612 are both methyl;
such as, for example, any selected from 6-(4-dimethylaminomethyl-phenyl)-pyridin-3-yl and 6-(3-dimethylaminomethyl-phenyl)-pyridin-3-yl.

Yet as exemplary R61-substituted Ah1 radicals may be more detailed mentioned, for example, 6-[3-(R61)-phenyl]-pyridin-3-yl or 6-[4-(R61)-phenyl]-pyridin-3-yl, in which
R61 is -O-T3-N(R613)R614, in which T3 is dimethylene or trimethylene, and
R613 and R614 together and with inclusion of the nitrogen atom, to which they are bonded, form a morpholino, piperidino, pyrrolidino or 4N-methyl-piperazino radical;
such as e.g. 6-[4-(2-pyrrolidin-1-yl-ethoxy)-phenyl]-pyridin-3-yl.

An example of R61-substituted Ah1 radicals may be [4-(R61)-phenyl]-pyridinyl, e.g. 6-[4-(R61)-phenyl]-pyridin-3-yl, in which R61 is any one selected from the group G_{Ah1} consisting of 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylaminoethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl.

Another example of R61-substituted Ah1 radicals may be [3-(R61)-phenyl]-pyridinyl, e.g. 6-[3-(R61)-phenyl]-pyridin-3-yl, in which R61 is any one selected from the group G_{Ah1} given above. A further example of R61-substituted Ah1 radicals may be [4-(R61)-phenyl]-thiophenyl, e.g. 5-[4-(R61)-phenyl]-thiophen-2-yl, in which R61 is any one selected from the group G_{Ah1} given above. Another example of R61-substituted Ah1 radicals may be [3-(R61)-phenyl]-thiophenyl, e.g. 5-[3-(R61)-phenyl]-thiophen-2-yl, in which R61 is any one selected from the group G_{Ah1} given above.

Specifically, as an exemplary R61-substituted Ah1 radical may be explicitely mentioned, for example, any one selected from 5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 5-[4-(2-morpholin-4-yl-ethoxy)-phenyl]-thiophen-2-yl, 5-[4-(3-morpholin-4-yl-propoxy)-phenyl]-thiophen-2-yl, 5-{4-[2-(4-methyl-piperazin-1-yl)-ethoxy]-phenyl}-thiophen-2-yl, 5-[4-(2-pyrrolidin-1-yl-ethoxy)-phenyl]-thiophen-2-yl, 5-(4-dimethylaminomethyl-phenyl)-thiophen-2-yl, 5-(3-dimethylaminomethyl-phenyl)-thiophen-2-yl, 6-(4-dimethylaminomethyl-phenyl)-pyridin-3-yl, 6-(3-dimethylaminomethyl-phenyl)-pyridin-3-yl, and 6-[4-(2-pyrrolidin-1-yl-ethoxy)-phenyl]-pyridin-3-yl, 5-(3-aminomethyl-phenyl)-thiophen-2-yl, 5-[3-(acetylamino)-methyl-phenyl]-thiophen-2-yl, 5-[3-(methylsulphonylamino)-methyl-phenyl]-thiophen-2-yl, and 5-(4-dimethylsulphamoyl-phenyl)-thiophen-2-yl.

More specifically, as an exemplary R61-substituted Ah1 radical may be more explicitely mentioned, for example, 5-(4-dimethylaminomethyl-phenyl)-thiophen-2-yl.

It is to be stated, that each of the radicals Hh1 and Ah1 is bonded via a ring carbon atom to the moiety T1.

Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group.

A particular embodiment of said Ha1 radicals refers to heteroaryl-phenyl radicals, particularly 3-(heteroaryl)-phenyl or 4-(heteroaryl)-phenyl radicals.

Ha1 may include, without being restricted thereto, the furanyl-phenyl, thiophenyl-phenyl, pyrazolyl-phenyl e.g. pyrazol-1-yl-phenyl or pyrazol-4-yl-phenyl, imidazolyl-phenyl e.g. imidazol-1-yl-phenyl, isoxazolyl-phenyl, or pyridinyl-phenyl radicals, or the thiazolyl-phenyl e.g. thiazol-4-yl-phenyl radical.

In a special detail, exemplary Ha1 radicals may include pyrazolyl-phenyl, e.g. 3-(pyrazolyl)-phenyl or 4-(pyrazolyl)-phenyl. Yet in a special detail, exemplary Ha1 radicals may include pyridinyl-phenyl, e.g. 4-(pyridinyl)-phenyl or 3-(pyridinyl)-phenyl. Yet in a special detail, exemplary Ha1 radicals may include isoxazolyl-phenyl, e.g. 4-(isoxazolyl)-phenyl or 3-(isoxazolyl)-phenyl. Yet in a special detail, exemplary Ha1 radicals may include thiazolyl-phenyl, e.g. 4-(thiazolyl)-phenyl or 3-(thiazolyl)-phenyl.

In a further special detail, exemplary Ha1 radicals may include 3-(pyrazol-1-yl)-phenyl, 4-(pyrazol-1-yl)-phenyl, 4-(pyridin-4-yl)-phenyl, 3-(pyridin-4-yl)-phenyl, 4-(pyridin-3-yl)-phenyl, 3-(pyridin-3-yl)-phenyl, 4-(isoxazol-4-yl)-phenyl, 3-(isoxazol-4-yl)-phenyl, 3-(pyrazol-4-yl)-phenyl or 4-(pyrazol-4-yl)-phenyl.

As non-limiting example of R61-substituted derivatives of Ha1 may be mentioned [1N-(1-4C-alkyl)-pyrazolyl]-phenyl, such as e.g. [1N-(1-4C-alkyl)-pyrazol-4-yl]-phenyl, like 3-[1N-(1-2C-alkyl)-pyrazol-4-yl]-phenyl or 4-[1N-(1-2C-alkyl)-pyrazol-4-yl]-phenyl, e.g. 3-(1N-methyl-pyrazol-4-yl)-phenyl or 4-(1N-methyl-pyrazol-4-yl)-phenyl.

As non-limiting example of R61- and/or R62-substituted derivatives of Ha1 may be mentioned (methyl-isoxazolyl)-phenyl or (dimethyl-isoxazolyl)-phenyl, such as e.g. 3-(3,5-dimethyl-isoxazol-4-yl)-phenyl or 4-(3,5-dimethyl-isoxazol-4-yl)-phenyl.

Yet as non-limiting example of R61-substituted derivatives of Ha1 may be mentioned [(R61)-pyridinyl]-phenyl, such as e.g. the following radicals:

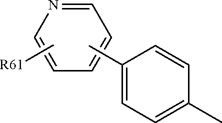

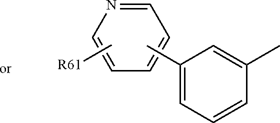

in which the substituent R61 can be attached in the ortho, or, in particular, meta or para position with respect to the binding position in which the pyridinyl ring is bonded to the phenyl radical, such as e.g. 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl.

As exemplary R61-substituted Ha1 radicals may be more detailed mentioned, for example, 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which
R61 is -T2-N(R611)R612, in which T2 is a bond, and
R611 and R612 together and with inclusion of the nitrogen atom, to which they are bonded, form a morpholino or 4N-methyl-piperazino, or a piperidino or pyrrolidino radical;
such as, for example, any selected from 4-[2-(4-methyl-piperazin-1-yl)-pyridin-4-yl]-phenyl and 3-[2-(4-methyl-piperazin-1-yl)-pyridin-4-yl]-phenyl.

Yet as exemplary R61-substituted Ha1 radicals may be more detailed mentioned, for example, 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which
R61 is -T2-N(R611)R612, in which T2 is a bond, and
R611 and R612 are both hydrogen;
such as, for example, any selected from 4-[6-amino-pyridin-3-yl]-phenyl and 3-[6-amino-pyridin-3-yl]-phenyl.

Yet as exemplary R61-substituted Ha1 radicals may be more detailed mentioned, for example, 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which R61 is methoxy; such as, for example, any selected from 4-[6-methoxy-pyridin-3-yl]-phenyl and 3-[6-methoxy-pyridin-3-yl]-phenyl.

Specifically, as an exemplary R61-substituted Ha1 radical may be explicitely mentioned, for example, any one selected from 4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, 3-[6-amino-pyridin-3-yl]-phenyl, 4-[6-methoxy-pyridin-3-yl]-phenyl, 3-[6-methoxy-pyridin-3-yl]-phenyl, 3-(1N-methyl-pyrazol-4-yl)-phenyl, 4-(1N-methyl-pyrazol-4-yl)-phenyl, and 4-(3,5-dimethyl-isoxazol-4-yl)-phenyl.

More specifically, as an exemplary R61-substituted Ha1 radical may be more explicitely mentioned, for example, any one selected from 4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, and 4-(1N-methyl-pyrazol-4-yl)-phenyl.

As part of the radicals Hh1, Ah1 and Ha1, the mentioned heteroaryl group selected from a group consisting of monocyclic 5- or 6-membered heteroaryl radicals comprising one or two heteroatoms, each of which is selected from the group consisting of nitrogen, oxygen and sulphur, may be choosen, for example, from the group consisting of, the 5-membered heteroaryl radicals, pyrrolyl, furanyl, thiophenyl, oxazolyl, isoxazolyl, thiazolyl, isothiazolyl, imidazolyl and pyrazolyl, and, the 6-membered heteroaryl radicals, pyridinyl, pyrimidinyl, pyrazinyl and pyridazinyl.

Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the to the parent molecular group. A particular embodiment of said Ha2 radicals refers to heteroaryl-phenyl radicals, particularly 3-(heteroaryl)-phenyl or 4-(heteroaryl)-phenyl radicals.

Another particular embodiment of said Ha2 radicals refers to heteroaryl-phenyl radicals, particularly 3-(heteroaryl)-phenyl or 4-(heteroaryl)-phenyl radicals, in which the heteroaryl moiety contains a benzene ring.

Another particular embodiment of said Ha2 radicals refers to heteroaryl-phenyl radicals, particularly 3-(heteroaryl)-phenyl or 4-(heteroaryl)-phenyl radicals, in which the heteroaryl moiety contains a benzene ring, and whereby the heteroaryl moiety is attached via said benzene ring to the phenyl moiety.

Ha2 may include, without being restricted thereto, the indolyl-phenyl, benzothiophenyl-phenyl, benzofuranyl-phenyl, benzoxazolyl-phenyl, benzothiazolyl-phenyl, indazolyl-phenyl, benzimidazolyl-phenyl, benzisoxazolyl-phenyl, benzisothiazolyl-phenyl, benzofurazanyl-phenyl, benzotriazolyl-phenyl, benzothiadiazolyl-phenyl, quinolinyl-phenyl, isoquinolinyl-phenyl, quinazolinyl-phenyl, quinoxalinyl-phenyl, cinnolinyl-phenyl, indolizinyl-phenyl or naphthyridinyl-phenyl.

In a special detail, exemplary Ha2 radicals may include 3-(indolyl)-phenyl or 4-(indolyl)-phenyl.

In a further special detail, exemplary Ha2 radicals may include 3-(indol-5-yl)-phenyl or 4-(indol-5-yl)-phenyl.

Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group, A particular embodiment of said Ha3 radicals refers to heteroaryl-phenyl radicals, particularly 3-(heteroaryl)-phenyl or 4-(heteroaryl)-phenyl radicals. Ha3 may include, without being restricted thereto, the thiadiazolyl-phenyl (e.g. [1,3,4]thiadiazol-2-yl-phenyl or [1,2,5]thiadiazol-3-yl-phenyl), oxadiazolyl-phenyl (e.g. [1,3,4]oxadiazol-2-yl-phenyl or [1,2,4]oxadiazol-5-yl-phenyl), triazolyl-phenyl (e.g. triazol-1-yl-phenyl or [1,2,3]triazol-4-yl) or tetrazolyl-phenyl (e.g. tetrazol-1-yl-phenyl or tetrazol-5-yl-phenyl) radicals.

In a special detail, exemplary Ha3 radicals may include triazolyl-phenyl, e.g. 3-(triazolyl)-phenyl or 4-(triazolyl)-phenyl. In a further special detail, exemplary Ha3 radicals may include 3-[1,2,3]triazol-4-yl-phenyl or 4-[1,2,3]triazol-4-yl-phenyl.

As non-limiting example of R61-substituted derivatives of Ha3 may be mentioned {1N-(R61)-[1,2,3]triazolyl}-phenyl, such as e.g. {1N-(R61)-[1,2,3]triazol-4-yl}-phenyl, like 3-{1N-(R61)-[1 ,2,3]triazol-4-yl}-phenyl or 4-{1 N-(R61)-[1 ,2,3]triazol-4-yl}-phenyl.

As exemplary R61-substituted Ha3 radicals may be more detailed mentioned, for example, 3-[1 N-(R61)-1 ,2,3-triazol-4-yl]-phenyl or 4-{1 N-(R61)-[1 ,2,3]triazol-4-yl}-phenyl, in which
R61 is -T2-N(R611)R612, in which
T2 is dimethylene or trimethylene, and
R611 and R612 together and with inclusion of the nitrogen atom, to which they are bonded, form a piperidino, pyrrolidino, morpholino or 4N-methyl-piperazino radical;
such as e.g. 4-{1-(2-morpholin-4-yl-ethyl)-[1,2,3]triazol-4-yl}-phenyl or 4-{1-(2-piperidin-1-yl-ethyl)-[1,2,3]triazol-4-yl}-phenyl.

Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group,

A particular embodiment of said Ha4 radicals refers to heteroaryl-phenyl radicals, particularly 3-(heteroaryl)-phenyl or 4-(heteroaryl)-phenyl radicals.

Another particular embodiment of said Ha4 radicals refers to heteroaryl-phenyl radicals, particularly 3-(heteroaryl)-phenyl or 4-(heteroaryl)-phenyl radicals, whereby the heteroaryl moiety is attached via its benzene ring to the phenyl moiety.

Ha4 may include, without being restricted thereto, the indolinyl-phenyl, isoindolinyl-phenyl, (1,2,3,4-tetrahydroquinolinyl)-phenyl or (1,2,3,4-tetrahydroisoquinolinyl)-phenyl, (2,3-dihydrobenzofuranyl)-phenyl, (2,3-dihydrobenzothiophenyl)-phenyl, (benzo[1,3]dioxolyl)-phenyl, (2,3-dihydrobenzo[1,4]dioxinyl)-phenyl, chromanyl-phenyl, chromenyl-phenyl or (2,3-dihydrobenzo[1,4]oxazinyl)-phenyl.

In a special detail, exemplary Ha4 radicals may include (benzo[1,3]dioxolyl)-phenyl, e.g. 3-(benzo[1,3]dioxolyl)-phenyl or 4-(benzo[1,3]dioxolyl)-phenyl, such as, for example, (benzo[1,3]dioxol-5-yl)-phenyl, e.g. 3-(benzo[1,3]dioxol-5-yl)-phenyl or 4-(benzo[1,3]dioxol-5-yl)-phenyl. Yet in a special detail, exemplary Ha4 radicals may include (2,3-dihydrobenzofuranyl)-phenyl, e.g. 3-(2,3-dihydrobenzofuranyl)-phenyl or 4-(2,3-dihydrobenzofuranyl)-phenyl, such as, for example, (2,3-dihydrobenzofuran-5-yl)-phenyl or (2,3-dihydrobenzofuran-6-yl)-phenyl, e.g. 3-(2,3-dihydrobenzofuran-5-yl)-phenyl or 4-(2,3-dihydrobenzofuran-5-yl)-phenyl. In a further special detail, exemplary Ha4 radicals may include 4-(2,3-dihydrobenzofuran-5-yl)-phenyl.

Har2 stands for a monocyclic 5- or 6-membered unsaturated heteroaromatic ring comprising one to three heteroatoms, each of which is selected from the group consisting of nitrogen, oxygen and sulfur.

Har2 may include, without being restricted thereto, thiophene, oxazole, isoxazole, thiazole, isothiazole, imidazole, pyrazole, triazole, thiadiazole, oxadiazole, pyridine, pyrimidine, pyrazine or pyridazine.

In a special detail, an exemplary Har2 radical may be pyridine.

Cyc1 stands for a ring system of formula Ia, which is bonded to the nitrogen atom of the carboxamide group via the moiety A. Cyc1 may include, without being restricted thereto, 2-aminophenyl substituted by R71 and/or R72. In a special detail, an exemplary Cyc1 radical may be 2-aminophenyl.

Naphthyl, alone or as part of another group, includes naphthalen-1-yl and naphthalen-2-yl.

In the meaning of the present invention, it is to be understood, that, when two structural portions of the compounds according to this invention are linked via a constituent which has the meaning “bond”, then said two portions are directly attached to another via a single bond.

When R61 has the meaning of -U-T3-N(R613)R614, in which U stands for —C(O)NH—, then R61 is the radical —C(O)NH—T3-N(R613)R614.

As it is known for the skilled person, the expressions morpholino, 4N-(1-4C-alkyl)-piperazino, pyrrolidino and the like stand for morpholin-4-yl, 4N-(1-4C-alkyl)-piperazin-1-yl, pyrrolidin-1-yl and the like, respectively.

In general, unless otherwise mentioned the heterocyclic groups mentioned herein refer to all of the possible isomeric forms thereof. The heterocyclic groups mentioned herein refer, unless otherwise noted, in particular to all of the possible positional isomers thereof. Thus, for example, the term pyridyl or pyridinyl, alone or as part of another group, includes pyridin-2-yl, pyridin-3-yl and pyridin-4-yl.

Constituents which are optionally substituted as stated herein, may be substituted, unless otherwise noted, at any possible position.

The carbocyclic groups, alone or as part of other groups, mentioned herein may be substituted by their given substituents or parent molecular groups, unless otherwise noted, at any substitutable ring carbon atom.

The heterocyclic groups, alone or as part of other groups, mentioned herein may be substituted by their given substituents or parent molecular groups, unless otherwise noted, at any possible position, such as e.g. at any substitutable ring carbon or ring nitrogen atom.

Rings containing quaternizable imino-type ring nitrogen atoms (—N═) may be particularly not quaternized on these imino-type ring nitrogen atoms by the mentioned substituents or parent molecular groups.

Any heteroatom of a heterocyclic ring with unsatisfied valences mentioned herein is assumed to have the hydrogen atom(s) to satisfy the valences.

When any variable occurs more than one time in any constituent, each definition is independent.

According to expert’s knowledge the compounds of formula I of the invention as well as their salts may contain, e.g. when isolated in crystalline form, varying amounts of solvents. Included within the scope of the invention are therefore all solvates and in particular all hydrates of the compounds of formula I as well as all solvates and in particular all hydrates of the salts of the compounds of formula I.

The substituents R61 and R62 of compounds of formula I can be attached in any possible position of the Aa1, Hh1, Ha1, Ha2, Ha3, Ha4 or Ah1 radical, whereby emphasis is given to the attachement at the terminal ring;
in another embodiment, Q1 is monosubstituted by R61, and is Aa1, Hh1, Ha1 or Ah1, whereby emphasis is given to the attachement of R61 at the terminal ring;
in yet another embodiment, R6 is Aa1, Ha1 or Ha2, each of which is monosubstituted by R61, whereby emphasis is given to the attachement of R61 at the terminal ring;
in yet another embodiment, R6 is Aa1, Hh1, Ha1, Ha2 or Ah1, each of which is monosubstituted by R61, whereby emphasis is given to the attachement of R61 at the terminal ring;
in yet another embodiment, R6 is Aa1, Hh1, Ha1, Ha2, Ha3 or Ah1, each of which is monosubstituted by R61, whereby emphasis is given to the attachement of R61 at the terminal ring;
in yet another embodiment, R6 is Ha2, Ha3 or Ha4, each of which is unsubstituted.

Within the meaning of this invention, the terminal ring of Aa1, Hh1, Ha1, Ha2, Ha3, Ha4 or Ah1 refers to those ring portion of these radicals which is not directly attached to the T1 moiety.

The person skilled in the art is aware on account of his/her expert knowledge that certain combinations of the variable characteristics mentioned in the description of this invention may lead to chemically les stable compounds. This can apply, for example, to certain compounds, in which -in a manner being disadvantageous for chemical stability- two heteroatoms (S, N or O) would directly meet or would only be separated by one carbon atom. Particularly, the compounds according to this invention are those, in which the combination of the abovementioned variable substituents does not lead to chemically less stable compounds.

Compounds according to aspect A of the present invention more worthy to be mentioned are those compounds of formula I in which
R1, R2, R3, R4 and R5 are independently hydrogen or 1-4C-alkyl,
R6 is -T1-Q1, in which T1 is a bond, either
Q1 is substituted by R61 and/or R62, and is Aa1, Hh1, Ha1, Ha2, Ha3, Ha4 or Ah1,
Or Q1 is unsubstituted, and is Ha2, Ha3 or Ha4,
in which
R61 is 1-4C-alkyl, 1-4C-alkoxy, hydroxyl, trifluoromethyl, halogen, hydroxy-1-4C-alkyl, 1-4C-alkylsulphonylamino, tolylsulphonylamino, phenylsulphonylamino, 1-4C-alkylcarbonylamino, di-1-4C-alkylaminosulphonyl, -T2-N(R611)R612, -U-T3-N(R613)R614, -T4-Het3, or -V-T5-Het4, in which T2 is a bond or 1-4C-alkylene,
R611 is hydrogen, 1-4C-alkyl, 3-7C-cycloalkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 1-4C-alkoxy-2-4C-alkyl, 1-4C-alkylcarbonyl, or 1-4C-alkylsulphonyl,
R614 is hydrogen or 1-4C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
T4 is a bond or 1-4C-alkylene,
Het3 is 1 N-(1-4C-alkyl)-piperidinyl or 1 N-(1-4C-alkyl)-pyrrolidinyl,
V is —O— (oxygen) or —C(O)NH—,
T5 is a bond, or 1-4C-alkylene,
Het4 is 1 N-(1-4C-alkyl)-piperidinyl or 1 N-(1-4C-alkyl)-pyrrolidinyl,
R62 is 1-4C-alkyl,
Aa1 is biphenyl,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
Ha4 is a heteroaryl-phenyl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and a phenyl group, whereby said heteroaryl and phenyl groups are linked together via a single bond, and whereby Ha4 is bonded via said phenyl moiety to the to the parent molecular group,
R7 is hydroxyl, or 2-aminophenyl,
and the salts of these compounds.

Compounds according to aspect A of the present invention in particular worthy to be mentioned are those compounds of formula I in which
R1, R2, R3, R4 and R5 are hydrogen,
R6 is -T1-Q1, in which T1 is a bond, either
Q1 is substituted by R61 and/or R62 on the terminal ring, and is Aa1, Hh1, Ha1, Ha2, Ha3, Ha4 or Ah1,
Or Q1 is unsubstituted, and is Ha2, Ha3 or Ha4,
in which
R61 is 1-2C-alkyl, 1-2C-alkoxy, hydroxyl, trifluoromethyl, halogen, hydroxy-1-2C-alkyl, 1-2C-alkylsulphonylamino, 1-2C-alkylcarbonylamino, di-1-2C-alkylaminosulphonyl, -T2-N(R611)R612, -U-T3-N(R613)R614, -T4-Het3, or -V-T5-Het4, in which
T2 is a bond or straight chain 1-4C-alkylene,
R611 is hydrogen, 1-2C-alkyl, 3-5C-cycloalkyl, 1-2C-alkoxy-2-3C-alkyl, 1-2C-alkylcarbonyl, or 1-2C-alkylsulphonyl,
R612 is hydrogen or 1-2C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-2C-alkyl)-piperazino,
U is —O— (oxygen) or —C(O)NH—,
T3 is straight chain 2-4C-alkylene,
R613 is hydrogen, 1-2C-alkyl, 3-5C-cycloalkyl, 1-2C-alkoxy-2-3C-alkyl, 1-2C-alkylcarbonyl, or 1-2C-alkylsulphonyl,
R614 is hydrogen or 1-2C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-2C-alkyl)-piperazino,
T4 is a bond or straight chain 1-4C-alkylene,
Het3 is 1N-(1-2C-alkyl)-piperidinyl or 1N-(1-2C-alkyl)-pyrrolidinyl,
V is —O— (oxygen) or —C(O)NH—,
T5 is a bond or straight chain 1-4C-alkylene,
Het4 is 1 N-(1-2C-alkyl)-piperidinyl or 1 N-(1-2C-alkyl)-pyrrolidinyl,
R62 is 1-2C-alkyl,
Aa1 is 1,1′-biphenyl-3-yl or 1,1′-biphenyl-4-yl,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a 3-(heteroaryl)-phenyl or 4-(heteroaryl)-phenyl radical each made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a 3-(heteroaryl)-phenyl or 4-(heteroaryl)-phenyl radical each made up of a heteroaryl group selected from a group consisting of fused bicyclic 9- or 10-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a 3-(heteroaryl)-phenyl or 4-(heteroaryl)-phenyl radical each made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
Ha4 is a 3-(heteroaryl)-phenyl or 4-(heteroaryl)-phenyl radical each made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and a phenyl group, whereby said heteroaryl and phenyl groups are linked together via a single bond, and whereby Ha4 is bonded via said phenyl moiety to the to the parent molecular group,
R7 is hydroxyl, or 2-aminophenyl,
and the salts of these compounds.

Compounds according to aspect A of the present invention in more particular worthy to be mentioned are those compounds of formula I in which
R1, R2, R3, R4 and R5 are hydrogen,
R6 is -T1-Q1, in which T1 is a bond, either
Q1 is substituted by R61 and/or R62 on the terminal ring, and is Aa1, Hh1, Ha1, Ha2, Ha3, Ha4 or Ah1,
or Q1 is unsubstituted, and is Ha2, Ha3 or Ha4,
in which
R61 is methyl, methoxy, hydroxyl, trifluoromethyl, hydroxymethyl, methylsulphonylamino, methylcarbonylamino, dimethylaminosulphonyl, -T2-N(R611)R612, -U-T3-N(R613)R614, -T4-Het3, or -V-T5-Het4, in which
T2 is a bond, methylene, dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4-methyl-piperazino,
U is —O— (oxygen) or —C(O)NH—,
T3 is dimethylene or trimethylene,
R613 is hydrogen, methyl, cyclopropyl, cyclopentyl, 2-methoxyethyl, acetyl or methylsulphonyl,
R614 is hydrogen or methyl,
or R613 and R614 together and with inclusion of the nitrogen atom, to which they are bonded, form a heterocyclic ring Het2, in which
Het2 is morpholino, piperidino, pyrrolidino, piperazino or 4-methyl-piperazino,
T4 is a bond, methylene, dimethylene or trimethylene,
Het3 is 1-methyl-piperidinyl or 1-methyl-pyrrolidinyl,
V is —O— (oxygen) or —C(O)NH—,
T5 is a bond, methylene, dimethylene or trimethylene,
Het4 is 1-methyl-piperidinyl or 1-methyl-pyrrolidinyl,
R62 is methyl,
Aa1 is 1,1′-biphenyl-3-yl, or 1,1′-biphenyl-4-yl,
Hh1 is a bisheteroaryl radical made up of two heteroaryl groups, which are selected independently from a group consisting of pyrrolyl, furanyl, thiophenyl, oxazolyl, isoxazolyl, thiazolyl, isothiazolyl, imidazolyl, pyrazolyl, pyridinyl, pyrimidinyl, pyrazinyl and pyridazinyl, and which are linked together via a single bond,
such as, for example,
Hh1 is pyridinyl-thiophenyl, thiazolyl-thiophenyl, pyrazolyl-thiophenyl, bipyridyl, pyrazolyl-pyridinyl, or thiazolyl-pyridinyl,
Ah1 is a phenyl-heteroaryl radical made up of an phenyl group and a heteroaryl group selected from a group consisting of pyrrolyl, furanyl, thiophenyl, oxazolyl, isoxazolyl, thiazolyl, isothiazolyl, imidazolyl, pyrazolyl, pyridinyl, pyrimidinyl, pyrazinyl and pyridazinyl, whereby said phenyl and heteroaryl groups are linked together via a single bond, and whereby Ah1 is bonded via said heteroaryl moiety to the parent molecular group,
such as, for example,
Ah1 is phenyl-thiophenyl, or phenyl-pyridinyl,
Ha1 is a 3-(heteroaryl)-phenyl or 4-(heteroaryl)-phenyl radical each made up of a heteroaryl group selected from a group consisting of pyrrolyl, furanyl, thiophenyl, oxazolyl, isoxazolyl, thiazolyl, isothiazolyl, imidazolyl, pyrazolyl, pyridinyl, pyrimidinyl, pyrazinyl and pyridazinyl, and a phenyl group, whereby said heteroaryl and phenyl groups are linked together via a single bond, and whereby Ha1 is bonded via said phenyl moiety to the to the parent molecular group,
such as, for example,
Ha1 is 3-(pyridinyl)-phenyl, 3-(thiazolyl)-phenyl, 3-(pyrazolyl)-phenyl, 3-(isoxazolyl)-phenyl, 4-(pyridinyl)-phenyl, 4-(thiazolyl)-phenyl, 4-(pyrazolyl)-phenyl, or 4-(isoxazolyl)-phenyl,
Ha2 is a 3-(heteroaryl)-phenyl or 4-(heteroaryl)-phenyl radical each made up of a heteroaryl group selected from a group consisting of indolyl, benzothiophenyl, benzofuranyl, benzoxazolyl, benzothiazolyl, indazolyl, benzimidazolyl, benzisoxazolyl, benzisothiazolyl, benzofurazanyl, benzotriazolyl, benzothiadiazolyl, quinolinyl, isoquinolinyl, quinazolinyl, quinoxalinyl, cinnolinyl, indolizinyl and naphthyridinyl, and a phenyl group, whereby said heteroaryl and phenyl groups are linked together via a single bond, and whereby Ha2 is bonded via said phenyl moiety to the to the parent molecular group,
such as, for example,
Ha2 is 3-(indolyl)-phenyl, or 4-(indolyl)-phenyl,
Ha3 is a 3-(heteroaryl)-phenyl or 4-(heteroaryl)-phenyl radical each made up of a heteroaryl group selected from a group consisting of thiadiazolyl, oxadiazolyl, triazolyl and tetrazolyl, and a phenyl group, whereby said heteroaryl and phenyl groups are linked together via a single bond, and whereby Ha3 is bonded via said phenyl moiety to the to the parent molecular group,
such as, for example,
Ha3 is 3-(triazolyl)-phenyl, or 4-(triazolyl)-phenyl,
Ha4 is a 3-(heteroaryl)-phenyl or 4-(heteroaryl)-phenyl radical each made up of a heteroaryl group selected from a group consisting of indolinyl, isoindolinyl, 1,2,3,4-tetrahydroquinolinyl, 1,2,3,4-tetrahydroisoquinolinyl, 2,3-dihydrobenzofuranyl, 2,3-dihydrobenzothiophenyl, benzo[1,3]dioxolyl, 2,3-dihydrobenzo[1,4]dioxinyl, chromanyl, chromenyl and 2,3-dihydrobenzo[1,4]oxazinyl, and a phenyl group, whereby said heteroaryl and phenyl groups are linked together via a single bond, and whereby Ha3 is bonded via said phenyl moiety to the to the parent molecular group,
such as, for example,
Ha4 is 3-(benzo[1,3]dioxolyl)-phenyl, 4-(benzo[1,3]dioxolyl)-phenyl, 3-(2,3-dihydrobenzofuranyl)-phenyl, or 4-(2,3-dihydrobenzofuranyl)-phenyl,
R7 is hydroxyl, or 2-aminophenyl,
and the salts of these compounds.

Compounds according to aspect A of the present invention to be emphasized are those compounds of formula I in which
R1, R2, R3, R4 and R5 are hydrogen,
R6 is -T1-Q1, in which T1 is a bond; either
Q1 is substituted by R61 on the terminal ring, and is Aa1 or Ah1, in which
Aa1 is 1,1′-biphenyl-3-yl, or 1,1′-biphenyl-4-yl,
such as, for example,
   3′-(R61)-1,1′-biphenyl-3-yl, 4′-(R61)-1,1′-biphenyl-3-yl, 3′-(R61)-1,1′-biphenyl-4-yl or
   4′-(R61)-1,1′-biphenyl-4-yl,
Ah1 is phenyl-thiophenyl, or phenyl-pyridinyl,
such as, for example,
   [3-(R61)-phenyl]-thiophenyl, [4-(R61)-phenyl]-thiophenyl, [3-(R61)-phenyl]-pyridinyl or [4-(R61)-phenyl]-pyridinyl,
   e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl,
in which
R61 is methoxy, hydroxyl, trifluoromethyl, hydroxymethyl, methylsulphonylamino, methylcarbonylamino, dimethylaminosulphonyl, -T2-N(R611)R612, -U-T3-N(R613)R614, -T4-Het3, or -V-T5-Het4, in which
T2 is a bond, methylene, dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
U is —O— (oxygen) or —C(O)NH—,
T3 is dimethylene or trimethylene,
R613 and R614 are methyl,
or R613 and R614 together and with inclusion of the nitrogen atom, to which they are bonded, form a heterocyclic ring Het2, in which Het2 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl,
V is —O— (oxygen) or —C(O)NH—,
T5 is a bond, methylene, dimethylene or trimethylene,
Het4 is 1-methyl-piperidinyl or 1-methyl-pyrrolidinyl; or
Q1 is substituted by R61 on the terminal ring, and is Hh1 or Ha1, in which
Hh1 is pyridinyl-thiophenyl, or bipyridyl,
such as, for example,
   [2-(R61)-pyridin-4-yl]-thiophenyl or [6-(R61)-pyridin-3-yl]-thiophenyl, e.g. 5-[2-(R61)-pyridin-4-yl]-thiophen-2-yl or 5-[6-(R61)-pyridin-3-yl]-thiophen-2-yl, or
   [2-(R61)-pyridin-4-yl]-pyridinyl or [6-(R61)-pyridin-3-yl]-pyridinyl, e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,
such as, for example,
   3-[2-(R61)-pyridin-4-yl]-phenyl, 3-[6-(R61)-pyridin-3-yl]-phenyl, 4-[2-(R61)-pyridin-4-yl]-phenyl or 4-[6-(R61)-pyridin-3-yl]-phenyl,
in which
R61 is methoxy, or -T2-N(R611)R612, in which
T2 is a bond,
R611 and R612 are independently hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino; or
Q1 is 3-(1-methyl-pyrazolyl)-phenyl, 4-(1-methyl-pyrazolyl)-phenyl, 3-(methyl-thiazolyl)-phenyl, 4-(methyl-thiazolyl)-phenyl, 3-(dimethyl-isoxazolyl)-phenyl, 4-(dimethyl-isoxazolyl)-phenyl, (1-methyl-pyrazolyl)-thiophenyl, (1-methyl-pyrazolyl)-pyridinyl, (methyl-thiazolyl)-thiophenyl, (methyl-thiazolyl)-pyridinyl, 3-(benzo[1,3]dioxolyl)-phenyl, 4-(benzo[1,3]dioxolyl)-phenyl, 3-(2,3-dihydrobenzofuranyl)-phenyl, 4-(2,3-dihydrobenzofuranyl)-phenyl, 3-(1-methyl-indolyl)-phenyl, or 4-(1-methyl-indolyl)-phenyl,
such as, for example,
   3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl or 4-(1-methyl-indol-5-yl)-phenyl; or
Q1 is 3-[1N-(R61)-pyrazolyl]-phenyl, 4-[1N-(R61)-pyrazolyl]-phenyl, [1N-(R61)-pyrazolyl)-thiophenyl, [1N-(R61)-pyrazolyl)-pyridinyl, 3-[1N-(R61)-triazolyl]-phenyl, or 4-[1N-(R61)-triazolyl]-phenyl,
such as, for example,
   3-[1 N-(R61)-pyrazol-4-yl]-phenyl, 4-[1 N-(R61)-pyrazol-4-yl]-phenyl, [1 N-(R61)-pyrazol-4-yl)-thiophenyl e.g. 5-[1N-(R61)-pyrazol-4-yl)-thiophen-2-yl, [1N-(R61)-pyrazol-4-yl)-pyridinyl e.g. 2-[1N-(R61)-pyrazol-4-yl)-pyridin-4-yl or 6-[1N-(R61)-pyrazol-4-yl)-pyridin-3-yl, 3-[1 N-(R61)-triazol-4-yl]-phenyl or 4-[1 N-(R61)-triazol-4-yl]-phenyl,
in which
R61 is -T2-N(R611)R612, or -T4-Het3, in which
T2 is dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl;
R7 is hydroxyl;
and the salts of these compounds.

Other compounds according to aspect A of the present invention to be emphasized are those compounds of formula I in which
R1, R2, R3, R4 and R5 are hydrogen,
R6 is -T1-Q1, in which
T1 is a bond;
either
Q1 is substituted by R61 on the terminal ring, and is Aa1 or Ah1, in which
Aa1 is 1,1′-biphenyl-3-yl, or 1,1′-biphenyl-4-yl,
such as, for example,
   3′-(R61)-1,1′-biphenyl-3-yl, 4′-(R61)-,1′-biphenyl-3-yl, 3′-(R61)-1,1′-biphenyl-4-yl or
   4′-(R61)-1,1′-biphenyl-4-yl,
Ah1 is phenyl-thiophenyl, or phenyl-pyridinyl,
such as, for example,
   [3-(R61)-phenyl]-thiophenyl, [4-(R61)-phenyl]-thiophenyl, [3-(R61)-phenyl]-pyridinyl or [4-(R61)-phenyl]-pyridinyl,
   e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl,
in which
R61 is methoxy, hydroxyl, trifluoromethyl, hydroxymethyl, methylsulphonylamino, methylcarbonylamino, dimethylaminosulphonyl, -T2-N(R611)R612, -U-T3-N(R613)R614, -T4-Het3, or -V-T5-Het4, in which
T2 is a bond, methylene, dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
U is —O— (oxygen) or —C(O)NH—,
T3 is dimethylene or trimethylene,
R613 is methyl,
R614 is methyl,
or R613 and R614 together and with inclusion of the nitrogen atom, to which they are bonded, form a heterocyclic ring Het2, in which
Het2 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl,
V is —O— (oxygen) or —C(O)NH—,
T5 is a bond, methylene, dimethylene or trimethylene,
Het4 is 1-methyl-piperidinyl or 1-methyl-pyrrolidinyl; or
Q1 is substituted by R61 on the terminal ring, and is Hh1 or Ha1, in which
Hh1 is pyridinyl-thiophenyl, or bipyridyl,
such as, for example,
   [2-(R61)-pyridin-4-yl]-thiophenyl or [6-(R61)-pyridin-3-yl]-thiophenyl, e.g. 5-[2-(R61)-pyridin-4-yl]-thiophen-2-yl or 5-[6-(R61)-pyridin-3-yl]-thiophen-2-yl, or
   [2-(R61)-pyridin-4-yl]-pyridinyl or [6-(R61)-pyridin-3-yl]-pyridinyl, e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,
such as, for example,
   3-[2-(R61)-pyridin-4-yl]-phenyl, 3-[6-(R61)-pyridin-3-yl]-phenyl, 4-[2-(R61)-pyridin-4-
   yl]-phenyl or 4-[6-(R61)-pyridin-3-yl]-phenyl,
in which
R61 is methoxy, or -T2-N(R611)R612, in which
T2 is a bond,
R611 is hydrogen or methyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino; or
Q1 is 3-(1-methyl-pyrazolyl)-phenyl, 4-(1-methyl-pyrazolyl)-phenyl, 3-(methyl-thiazolyl)-phenyl, 4-(methyl-thiazolyl)-phenyl, 3-(dimethyl-isoxazolyl)-phenyl, 4-(dimethyl-isoxazolyl)-phenyl, (1-methyl-pyrazolyl)-thiophenyl, (1-methyl-pyrazolyl)-pyridinyl, (methyl-thiazolyl)-thiophenyl, (methyl-thiazolyl)-pyridinyl, 3-(benzo[1,3]dioxolyl)-phenyl, 4-(benzo[1,3]dioxolyl)-phenyl, 3-(2,3-dihydrobenzofuranyl)-phenyl, 4-(2,3-dihydrobenzofuranyl)-phenyl, 3-(1-methyl-indolyl)-phenyl, or 4-(1-methyl-indolyl)-phenyl,
such as, for example,
   3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl or 4-(1-methyl-indol-5-yl)-phenyl; or
Q1 is 3-[1N-(R61)-pyrazolyl]-phenyl, 4-[1N-(R61)-pyrazolyl]-phenyl, [1N-(R61)-pyrazolyl)-thiophenyl, [1N-(R61)-pyrazolyl)-pyridinyl, 3-[1N-(R61)-triazolyl]-phenyl, or 4-[1N-(R61)-triazolyl]-phenyl,
such as, for example,
   3-[1 N-(R61)-pyrazol-4-yl]-phenyl, 4-[1 N-(R61)-pyrazol-4-yl]-phenyl, [1 N-(R61)-pyrazol-4-yl)-thiophenyl e.g. 5-[1N-(R61)-pyrazol-4-yl)-thiophen-2-yl, [1N-(R61)-pyrazol-4-yl)-pyridinyl e.g. 2-[1N-(R61)-pyrazol-4-yl)-pyridin-4-yl or 6-[1N-(R61)-pyrazol-4-yl)-pyridin-3-yl, 3-[1 N-(R61)-triazol-4-yl]-phenyl or 4-[1 N-(R61)-triazol-4-yl]-phenyl,
in which
R61 is -T2-N(R611)R612, or -T4-Het3, in which
T2 is dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl;
R7 is 2-aminophenyl;
and the salts of these compounds.

Compounds according to aspect A of the present invention to be more emphasized are those compounds of formula I in which
R1, R2, R3, R4 and R5 are hydrogen,
R6 is -T1-Q1, in which T1 is a bond; either
Q1 is substituted by R61 on the terminal ring, and is Aa1 or Ah1, in which
Aa1 is 1,1′-biphenyl-3-yl, or 1,1′-biphenyl-4-yl,
such as, for example,
   3′-(R61)-1,1′-biphenyl-3-yl, 4′-(R61)-1,1′-biphenyl-3-yl, 3′-(R61)-1,1′-biphenyl-4-yl or 4′-(R61)-1,1′-biphenyl-4-yl,
Ah1 is phenyl-thiophenyl, or phenyl-pyridinyl,
such as, for example,
   [3-(R61)-phenyl]-thiophenyl, [4-(R61)-phenyl]-thiophenyl, [3-(R61)-phenyl]-pyridinyl or [4-(R61)-phenyl]-pyridinyl,
   e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl,
in which
R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl; or
Q1 is substituted by R61 on the terminal ring, and is Hh1 or Ha1, in which
Hh1 is pyridinyl-thiophenyl, or bipyridyl,
such as, for example,
   [2-(R61)-pyridin-4-yl]-thiophenyl or [6-(R61)-pyridin-3-yl]-thiophenyl,
   e.g. 5-[2-(R61)-pyridin-4-yl]-thiophen-2-yl or 5-[6-(R61)-pyridin-3-yl]-thiophen-2-yl, or
   [2-(R61)-pyridin-4-yl]-pyridinyl or [6-(R61)-pyridin-3-yl]-pyridinyl, e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,
such as, for example,
   3-[2-(R61)-pyridin-4-yl]-phenyl, 3-[6-(R61)-pyridin-3-yl]-phenyl, 4-[2-(R61)-pyridin-4-yl]-phenyl or 4-[6-(R61)-pyridin-3-yl]-phenyl,
in which
R61 is any one selected from methylsulphonylamino, acetamido, amino, dimethylamino, morpholino, piperidino, pyrrolidino, 4-methyl-piperazino, hydroxy, trifluoromethyl and methoxy; or
Q1 is 3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl, or 4-(1-methyl-indol-5-yl)-phenyl; or
Q1 is 3-[1N-(R61)-pyrazol-4-yl]-phenyl, 4-[1N-(R61)-pyrazol-4-yl]-phenyl, [1N-(R61)-pyrazol-4-yl)-thiophenyl e.g. 5-[1N-(R61)-pyrazol-4-yl)-thiophen-2-yl, [1N-(R61)-pyrazol-4-yl)-pyridinyl e.g. 2-[1N-(R61)-pyrazol-4-yl)-pyridin-4-yl or 6-[1N-(R61)-pyrazol-4-yl)-pyridin-3-yl, 3-[1 N-(R61)-triazol-4-yl]-phenyl, or 4-[1 N-(R61)-triazol-4-yl]-phenyl,
in which
R61 is any one selected from 3-morpholin-4-yl-propyl, 2-morpholin-4-yl-ethyl, 3-(4-methyl-piperazin-1-yl)-propyl, 2-(4-methyl-piperazin-1-yl)-ethyl, 3-pyrrolidin-1-yl-propyl, 2-pyrrolidin-1-yl-ethyl, 3-piperidin-1-yl-propyl, 2-piperidin-1-yl-ethyl, 2-dimethylaminoethyl and 3-dimethylamino-propyl;
R7 is hydroxyl;
and the salts of these compounds.

Compounds according to aspect A of the present invention to be more emphasized are those compounds of formula I in which
R1, R2, R3, R4 and R5 are hydrogen,
R6 is -T1-Q1, in which T1 is a bond; either
Q1 is substituted by R61 on the terminal ring, and is Aa1 or Ah1, in which
Aa1 is 1,1′-biphenyl-3-yl, or 1,1′-biphenyl-4-yl,

such as, for example,
   3′-(R61)-1,1′-biphenyl-3-yl, 4′-(R61)-1,1′-biphenyl-3-yl, 3′-(R61)-1,1′-biphenyl-4-yl or
   4′-(R61)-1,1′-biphenyl-4-yl,
Ah1 is phenyl-thiophenyl, or phenyl-pyridinyl,
such as, for example,
   [3-(R61)-phenyl]-thiophenyl, [4-(R61)-phenyl]-thiophenyl, [3-(R61)-phenyl]-pyridinyl
   or [4-(R61)-phenyl]-pyridinyl,
   e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or
   6-[4-(R61)-phenyl]-pyridin-3-yl,
in which
R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl; or
Q1 is substituted by R61 on the terminal ring, and is Hh1 or Ha1, in which
Hh1 is pyridinyl-thiophenyl, or bipyridyl,
such as, for example,
   [2-(R61)-pyridin-4-yl]-thiophenyl or [6-(R61)-pyridin-3-yl]-thiophenyl,
   e.g. 5-[2-(R61)-pyridin-4-yl]-thiophen-2-yl or 5-[6-(R61)-pyridin-3-yl]-thiophen-2-yl, or
   [2-(R61)-pyridin-4-yl]-pyridinyl or [6-(R61)-pyridin-3-yl]-pyridinyl, e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,
such as, for example,
   3-[2-(R61)-pyridin-4-yl]-phenyl, 3-[6-(R61)-pyridin-3-yl]-phenyl, 4-[2-(R61)-pyridin-4-yl]-phenyl or 4-[6-(R61)-pyridin-3-yl]-phenyl,
in which
R61 is any one selected from methylsulphonylamino, acetamido, amino, dimethylamino, morpholino, piperidino, pyrrolidino, 4-methyl-piperazino, hydroxy, trifluoromethyl and methoxy; or
Q1 is 3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl, or 4-(1-methyl-indol-5-yl)-phenyl; or
Q1 is 3-[1N-(R61)-pyrazol-4-yl]-phenyl, 4-[1N-(R61)-pyrazol-4-yl]-phenyl, [1 N-(R61)-pyrazol-4-yl)-thiophenyl e.g. 5-[1 N-(R61)-pyrazol-4-yl)-thiophen-2-yl, [1N-(R61)-pyrazol-4-yl)-pyridinyl e.g. 2-[1N-(R61)-pyrazol-4-yl)-pyridin-4-yl or 6-[1N-(R61)-pyrazol-4-yl)-pyridin-3-yl, 3-[1 N-(R61)-triazol-4-yl]-phenyl, or 4-[1 N-(R61)-triazol-4-yl]-phenyl,
in which
R61 is any one selected from 3-morpholin-4-yl-propyl, 2-morpholin-4-yl-ethyl, 3-(4-methyl-piperazin-1-yl)-propyl, 2-(4-methyl-piperazin-1-yl)-ethyl, 3-pyrrolidin-1-yl-propyl, 2-pyrrolidin-1-yl-ethyl, 3-piperidin-1-yl-propyl, 2-piperidin-1-yl-ethyl, 2-dimethylaminoethyl and 3-dimethylamino-propyl;
R7 is 2-aminophenyl;
and the salts of these compounds.

Compounds according to aspect A of the present invention to be more emphasized are those compounds of formula I in which
R1, R2, R3, R4 and R5 are hydrogen,
R6 is -T1-Q1, in which T1 is a bond;
Q1 is any one selected from the group consisting of
3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl, 4′-(3-morpholin-4-yl-propyl)-biphenyl-4-yl, 3′-(3-morpholin-4-yl-propyl)-biphenyl-3-yl, 3′-(3-morpholin-4-yl-propyl)-biphenyl-4-yl, 4′-(4-methyl-piperazin-1-ylmethyl)-biphenyl-3-yl, 4′-(4-methyl-piperazin-1-ylmethyl)-biphenyl-4-yl, 3′-(4-methyl-piperazin-1-ylmethyl)-biphenyl-3-yl,
3′-(4-methyl-piperazin-1-ylmethyl)-biphenyl-4-yl, 4′-(2-morpholin-4-yl-ethoxy)-biphenyl-3-yl, 4′-(2-morpholin-4-yl-ethoxy)-biphenyl-4-yl, 3′-(2-morpholin-4-yl-ethoxy)-biphenyl-3-yl, 3′-(2-morpholin-4-yl-ethoxy)-biphenyl-4-yl, 4′-(3-morpholin-4-yl-propoxy)-biphenyl-3-yl, 4′-(3-morpholin-4-yl-propoxy)-biphenyl-4-yl, 3′-(3-morpholin-4-yl-propoxy)-biphenyl-3-yl, 3′-(3-morpholin-4-yl-propoxy)-biphenyl-4-yl, 4′-[2-(4-methyl-piperazin-1-yl)-ethoxy]-biphenyl-3-yl, 4′-[2-(4-methyl-piperazin-1-yl)-ethoxy]-biphenyl-4-yl, 3′-[2-(4-methyl-piperazin-1-yl)-ethoxy]-biphenyl-3-yl, 3′-[2-(4-methyl-piperazin-1-yl)-ethoxy]-biphenyl-4-yl, 4′-(2-pyrrolidin-1-yl-ethoxy]-biphenyl-3-yl, 4′-(2-pyrrolidin-1-yl-ethoxy]-biphenyl-4-yl, 3′-(2-pyrrolidin-1-yl-ethoxy]-biphenyl-3-yl, 3′-(2-pyrrolidin-1-yl-ethoxy]-biphenyl-4-yl, 3′-(3-pyrrolidin-1-yl-propoxy]-biphenyl-4-yl, 4′-(3-pyrrolidin-1-yl-propoxy]-biphenyl-4-yl, 3′-(3-pyrrolidin-1-yl-propoxy]-biphenyl-3-yl, 4′-(3-pyrrolidin-1-yl-propoxy]-biphenyl-3-yl, 4′-[3-(4-methyl-piperazin-1-yl)-propoxy]-biphenyl-4-yl, 3′-[3-(4-methyl-piperazin-1-yl)-propoxy]-biphenyl-4-yl, 4′-[3-(4-methyl-piperazin-1-yl)-propoxy]-biphenyl-3-yl, 3′-[3-(4-methyl-piperazin-1-yl)-propoxy]-biphenyl-3-yl,
4′-(2-(1-methyl-piperidin-4-yl)-ethoxy)-biphenyl-4-yl, 4′-(2-(1-methyl-piperidin-4-yl)-ethoxy)-biphenyl-3-yl,
3′-(2-(1-methyl-piperidin-4-yl)-ethoxy)-biphenyl-4-yl, 3′-(2-(1-methyl-piperidin-4-yl)-ethoxy)-biphenyl-3-yl,
2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl, 3′-dimethylaminomethyl-biphenyl-3-yl, 3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 3′-[(2-dimethylamino-ethylamino)-carbonyl]-biphenyl-3-yl, 2′-methylsulphonylamino-biphenyl-4-yl,
3′-methylsulphonylamino-biphenyl-4-yl, 4′-methylsulphonylamino-biphenyl-4-yl, 2′-methylsulphonylamino-biphenyl-3-yl, 3′-methylsulphonylamino-biphenyl-3-yl, 4′-methylsulphonylamino-biphenyl-3-yl, 4′-methylsulphonylamino-biphenyl-3-yl, 4′-dimethylsulphamoyl-biphenyl-4-yl, 4′-dimethylsulphamoyl-biphenyl-3-yl, 3′-dimethylsulphamoyl-biphenyl-4-yl, 3′-dimethylsulphamoyl-biphenyl-3-yl, 3′-acetamido-biphenyl-4-yl, 4′-acetamido-biphenyl-4-yl, 3′-acetamido-biphenyl-3-yl, 4′-acetamido-biphenyl-3-yl, 3′-amino-biphenyl-4-yl, 3′-dimethylamino-biphenyl-4-yl, 4′-morpholin-4-yl-biphenyl-4-yl,
4′-hydroxy-biphenyl-4-yl, 3′-trifluoromethyl-biphenyl-4-yl, 4′-methoxy-biphenyl-4-yl, 3′-aminobiphenyl-3-yl, 3′-dimethylamino-biphenyl-3-yl, 4′-morpholin-4-yl-biphenyl-3-yl, 4′-hydroxybiphenyl-3-yl, 3′-trifluoromethyl-biphenyl-3-yl, 4′-methoxy-biphenyl-3-yl, 4′-amino-biphenyl-4-yl, 4′-dimethylamino-biphenyl-4-yl, 3′-morpholin-4-yl-biphenyl-4-yl, 3′-hydroxy-biphenyl-4-yl,
4′-trifluoromethyl-biphenyl-4-yl, 3′-methoxy-biphenyl-4-yl, 4′-amino-biphenyl-3-yl, 4′-dimethylamino-biphenyl-3-yl, 3′-morpholin-4-yl-biphenyl-3-yl, 3′-hydroxy-biphenyl-3-yl, 4′-trifluoromethyl-biphenyl-3-yl and 3′-methoxy-biphenyl-3-yl,
4′-(2-methoxy-ethylamino)methyl-biphenyl-3-yl, 4′-(2-methoxy-ethylamino)methyl-biphenyl-4-yl,
3′-(2-methoxy-ethylamino)methyl-biphenyl-3-yl, 3′-(2-methoxy-ethylamino)methyl-biphenyl-4-yl,
4′-aminomethyl-biphenyl-3-yl, 4′-aminomethyl-biphenyl-4-yl, 3′-aminomethyl-biphenyl-3-yl, 3′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl, 4′-(acetylamino)-methyl-biphenyl-3-yl, 4′-(methylsulphonylamino)-methyl-biphenyl-3-yl, 3′-(acetylamino)-methyl-biphenyl-4-yl, 3′-(methylsulphonylamino)-methyl-biphenyl-4-yl, 4′-cyclopentylaminomethyl-biphenyl-4-yl, 4′-cyclopentylaminomethyl-biphenyl-3-yl, 3′-cyclopentylaminomethyl-biphenyl-4-yl, 3′-cyclopentylaminomethyl-biphenyl-3-yl, 4′-cyclopropylaminomethyl-biphenyl-3-yl, 4′-cyclopropylaminomethyl-biphenyl-4-yl, 3′-cyclopropylaminomethyl-biphenyl-3-yl, 3′-cyclopropylaminomethyl-biphenyl-4-yl, 3′-hydroxymethyl-biphenyl-4-yl, 3′-hydroxymethyl-biphenyl-3-yl,
4′-hydroxymethyl-biphenyl-4-yl, 4′-hydroxymethyl-biphenyl-3-yl, 5-[2-(4-methyl-piperazin-1-yl)-pyridin-4-yl]-thiophen-2-yl, 5-(1-methyl-pyrazol-4-yl)-thiophen-2-yl, 6-(1-methyl-pyrazol-4-yl)-pyridin-3-yl,
2′-(4-methyl-piperazin-1-yl)-2,4′-bipyridyl-5-yl, 5-(2-methyl-thiazol-4-yl)-thiophen-2-yl, 5-[4-(2-morpholin-4-yl-ethyl)-phenyl]-thiophen-2-yl, 5-[3-(2-morpholin-4-yl-ethyl)-phenyl]-thiophen-2-yl,
5-[4-(morpholin-4-yl-methyl)-phenyl]-thiophen-2-yl, 5-[3-(morpholin-4-yl-methyl)-phenyl]-thiophen-2-yl,
5-[4-(2-morpholin-4-yl-ethoxy)-phenyl]-thiophen-2-yl, 5-[3-(2-morpholin-4-yl-ethoxy)-phenyl]-thiophen-2-yl, 5-[4-(3-morpholin-4-yl-propoxy)-phenyl]-thiophen-2-yl, 5-[3-(3-morpholin-4-yl-propoxy)-phenyl]-thiophen-2-yl, 5-{4-[2-(4-methyl-piperazin-1-yl)-ethoxy]-phenyl}-thiophen-2-yl, 5-{3-[2-(4-methyl-piperazin-1-yl)-ethoxy]-phenyl}-thiophen-2-yl, 5-[4-(2-pyrrolidin-1-yl-ethoxy)-phenyl]-thiophen-2-yl, 5-[3-(2-pyrrolidin-1-yl-ethoxy)-phenyl]-thiophen-2-yl, 5-(4-dimethylaminomethyl-phenyl)-thiophen-2-yl, 5-(3-dimethylaminomethyl-phenyl)-thiophen-2-yl,
6-(4-dimethylaminomethyl-phenyl)-pyridin-3-yl, 6-(3-dimethylaminomethyl-phenyl)-pyridin-3-yl, 6-[4-(2-pyrrolidin-1-yl-ethoxy)-phenyl]-pyridin-3-yl, 6-[3-(2-pyrrolidin-1-yl-ethoxy)-phenyl]-pyridin-3-yl, 5-(3-aminomethyl-phenyl)-thiophen-2-yl, 5-[3-(acetylamino)-methyl-phenyl]-thiophen-2-yl, 5-[3-(methylsulphonylamino)-methyl-phenyl]-thiophen-2-yl, 5-(4-dimethylsulphamoyl-phenyl)-thiophen-2-yl, 5-(4-aminomethyl-phenyl)-thiophen-2-yl, 5-[4-(acetylamino)-methyl-phenyl]-thiophen-2-yl, 5-[4-(methylsulphonylamino)-methyl-phenyl]-thiophen-2-yl, 5-(3-dimethylsulphamoyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl, 4-(6-amino-pyridin-3-yl)-phenyl, 3-(6-amino-pyridin-3-yl)-phenyl, 4-(6-methoxy-pyridin-3-yl)-phenyl, 3-(6-methoxy-pyridin-3-yl)-phenyl, 3-(1-methyl-pyrazol-4-yl)-phenyl, 4-(1-methyl-pyrazol-4-yl)-phenyl, 4-(3,5-dimethyl-isoxazol-4-yl)-phenyl, 3-(3,5-dimethyl-isoxazol-4-yl)-phenyl, 4-(1-methyl-indol-5-yl)-phenyl, 3-(1-methyl-indol-5-yl)-phenyl,
4-{1-(2-morpholin-4-yl-ethyl)-[1,2,3]triazol-4-yl}-phenyl, 4-{1-(2-piperidin-1-yl-ethyl)-[1,2,3]triazol-4-yl}-phenyl, 3-{1-(2-morpholin-4-yl-ethyl)-[1,2,3]triazol-4-yl}-phenyl, 3-{1-(2-piperidin-1-yl-ethyl)-[1,2,3]triazol-4-yl}-phenyl, 4-(2,3-dihydrobenzofuran-5-yl)-phenyl, and 4-(benzo[1,3]dioxol-5-yl)-phenyl, 3-(2,3-dihydrobenzofuran-5-yl)-phenyl, and 3-(benzo[1,3]dioxol-5-yl)-phenyl,
R7 is hydroxyl,
and the salts of these compounds.

Compounds according to aspect A of the present invention to be more emphasized are those compounds of formula I in which
R1, R2, R3, R4 and R5 are hydrogen,
R6 is -T1-Q1, in which T1 is a bond;
Q1 is any one selected from the group consisting of
3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl, 4′-(3-morpholin-4-yl-propyl)-biphenyl-4-yl, 3′-(3-morpholin-4-yl-propyl)-biphenyl-3-yl, 3′-(3-morpholin-4-yl-propyl)-biphenyl-4-yl,
4′-(4-methyl-piperazin-1-ylmethyl)-biphenyl-3-yl, 4′-(4-methyl-piperazin-1-ylmethyl)-biphenyl-4-yl,
3′-(4-methyl-piperazin-1-ylmethyl)-biphenyl-3-yl, 3′-(4-methyl-piperazin-1-ylmethyl)-biphenyl-4-yl,
4′-(2-morpholin-4-yl-ethoxy)-biphenyl-3-yl, 4′-(2-morpholin-4-yl-ethoxy)-biphenyl-4-yl, 3′-(2-morpholin-4-yl-ethoxy)-biphenyl-3-yl, 3′-(2-morpholin-4-yl-ethoxy)-biphenyl-4-yl, 4′-(3-morpholin-4-yl-propoxy)-biphenyl-3-yl, 4′-(3-morpholin-4-yl-propoxy)-biphenyl-4-yl, 3′-(3-morpholin-4-yl-propoxy)-biphenyl-3-yl, 3′-(3-morpholin-4-yl-propoxy)-biphenyl-4-yl, 4′-[2-(4-methyl-piperazin-1-yl)-ethoxy]-biphenyl-3-yl, 4′-[2-(4-methyl-piperazin-1-yl)-ethoxy]-biphenyl-4-yl, 3′-[2-(4-methyl-piperazin-1-yl)-ethoxy]-biphenyl-3-yl, 3′-[2-(4-methyl-piperazin-1-yl)-ethoxy]-biphenyl-4-yl, 4′-(2-pyrrolidin-1-yl-ethoxy]-biphenyl-3-yl, 4′-(2-pyrrolidin-1-yl-ethoxy]-biphenyl-4-yl, 3′-(2-pyrrolidin-1-yl-ethoxy]-biphenyl-3-yl, 3′-(2-pyrrolidin-1-yl-ethoxy]-biphenyl-4-yl, 3′-(3-pyrrolidin-1-yl-propoxy]-biphenyl-4-yl, 4′-(3-pyrrolidin-1-yl-propoxy]-biphenyl-4-yl, 3′-(3-pyrrolidin-1-yl-propoxy]-biphenyl-3-yl, 4′-(3-pyrrolidin-1-yl-propoxy]-biphenyl-3-yl,
4′-[3-(4-methyl-piperazin-1-yl)-propoxy]-biphenyl-4-yl, 3′-[3-(4-methyl-piperazin-1-yl)-propoxy]-biphenyl-4-yl, 4′-[3-(4-methyl-piperazin-1-yl)-propoxy]-biphenyl-3-yl, 3′-[3-(4-methyl-piperazin-1-yl)-propoxy]-biphenyl-3-yl, 4′-(2-(1-methyl-piperidin-4-yl)-ethoxy)-biphenyl-4-yl, 4′-(2-(1-methyl-piperidin-4-yl)-ethoxy)-biphenyl-3-yl, 3′-(2-(1-methyl-piperidin-4-yl)-ethoxy)-biphenyl-4-yl, 3′-(2-(1-methyl-piperidin-4-yl)-ethoxy)-biphenyl-3-yl, 2′-dimethylaminomethyl-biphenyl-4-yl,
4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl, 3′-dimethylaminomethyl-biphenyl-3-yl, 3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 3′-[(2-dimethylamino-ethylamino)-carbonyl]-biphenyl-3-yl,
2′-methylsulphonylamino-biphenyl-4-yl, 3′-methylsulphonylamino-biphenyl-4-yl, 4′-methylsulphonylamino-biphenyl-4-yl, 2′-methylsulphonylamino-biphenyl-3-yl, 3′-methylsulphonylamino-biphenyl-3-yl, 4′-methylsulphonylamino-biphenyl-3-yl, 4′-methylsulphonylamino-biphenyl-3-yl,
4′-dimethylsulphamoyl-biphenyl-4-yl, 4′-dimethylsulphamoyl-biphenyl-3-yl, 3′-dimethylsulphamoyl-biphenyl-4-yl, 3′-dimethylsulphamoyl-biphenyl-3-yl, 3′-acetamido-biphenyl-4-yl, 4′-acetamido-biphenyl-4-yl, 3′-acetamido-biphenyl-3-yl, 4′-acetamido-biphenyl-3-yl, 3′-amino-biphenyl-4-yl, 3′-dimethylamino-biphenyl-4-yl, 4′-morpholin-4-yl-biphenyl-4-yl,
4′-hydroxy-biphenyl-4-yl, 3′-trifluoromethyl-biphenyl-4-yl, 4′-methoxy-biphenyl-4-yl, 3′-aminobiphenyl-3-yl, 3′-dimethylamino-biphenyl-3-yl, 4′-morpholin-4-yl-biphenyl-3-yl, 4′-hydroxybiphenyl-3-yl, 3′-trifluoromethyl-biphenyl-3-yl, 4′-methoxy-biphenyl-3-yl, 4′-amino-biphenyl-4-yl, 4′-dimethylamino-biphenyl-4-yl, 3′-morpholin-4-yl-biphenyl-4-yl, 3′-hydroxy-biphenyl-4-yl,
4′-trifluoromethyl-biphenyl-4-yl, 3′-methoxy-biphenyl-4-yl, 4′-amino-biphenyl-3-yl, 4′-dimethylamino-biphenyl-3-yl, 3′-morpholin-4-yl-biphenyl-3-yl, 3′-hydroxy-biphenyl-3-yl, 4′-trifluoromethyl-biphenyl-3-yl and 3′-methoxy-biphenyl-3-yl, 4′-(2-methoxy-ethylamino)methylbiphenyl-3-yl, 4′-(2-methoxy-ethylamino)methyl-biphenyl-4-yl, 3′-(2-methoxy-ethylamino)methyl-biphenyl-3-yl, 3′-(2-methoxy-ethylamino)methyl-biphenyl-4-yl, 4′-aminomethyl-biphenyl-3-yl, 4′-aminomethyl-biphenyl-4-yl, 3′-aminomethyl-biphenyl-3-yl, 3′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl, 4′-(acetylamino)-methyl-biphenyl-3-yl, 4′-(methylsulphonylamino)-methyl-biphenyl-3-yl, 3′-(acetylamino)-methyl-biphenyl-4-yl, 3′-(methylsulphonylamino)-methyl-biphenyl-4-yl, 4′-cyclopentylaminomethyl-biphenyl-4-yl, 4′-cyclopentylaminomethyl-biphenyl-3-yl, 3′-cyclopentylaminomethyl-biphenyl-4-yl, 3′-cyclopentylaminomethyl-biphenyl-3-yl, 4′-cyclopropylaminomethyl-biphenyl-3-yl, 4′-cyclopropylaminomethyl-biphenyl-4-yl, 3′-cyclopropylaminomethyl-biphenyl-3-yl, 3′-cyclopropylaminomethyl-biphenyl-4-yl, 3′-hydroxymethyl-biphenyl-4-yl, 3′-hydroxymethyl-biphenyl-3-yl,
4′-hydroxymethyl-biphenyl-4-yl, 4′-hydroxymethyl-biphenyl-3-yl, 5-[2-(4-methyl-piperazin-1-yl)-pyridin-4-yl]-thiophen-2-yl, 5-(1-methyl-pyrazol-4-yl)-thiophen-2-yl, 6-(1-methyl-pyrazol-4-yl)-pyridin-3-yl,
2′-(4-methyl-piperazin-1-yl)-2,4′-bipyridyl-5-yl, 5-(2-methyl-thiazol-4-yl)-thiophen-2-yl,
5-[4-(2-morpholin-4-yl-ethyl)-phenyl]-thiophen-2-yl, 5-[3-(2-morpholin-4-yl-ethyl)-phenyl]-thiophen-2-yl,
5-[4-(morpholin-4-yl-methyl)-phenyl]-thiophen-2-yl, 5-[3-(morpholin-4-yl-methyl)-phenyl]-thiophen-2-yl,
5-[4-(2-morpholin-4-yl-ethoxy)-phenyl]-thiophen-2-yl, 5-[3-(2-morpholin-4-yl-ethoxy)-phenyl]-thiophen-2-yl, 5-[4-(3-morpholin-4-yl-propoxy)-phenyl]-thiophen-2-yl, 5-[3-(3-morpholin-4-yl-propoxy)-phenyl]-thiophen-2-yl, 5-{4-[2-(4-methyl-piperazin-1-yl)-ethoxy]-phenyl}-thiophen-2-yl, 5-{3-[2-(4-methyl-piperazin-1-yl)-ethoxy]-phenyl}-thiophen-2-yl, 5-[4-(2-pyrrolidin-1-yl-ethoxy)-phenyl]-thiophen-2-yl, 5-[3-(2-pyrrolidin-1-yl-ethoxy)-phenyl]-thiophen-2-yl, 5-(4-dimethylaminomethyl-phenyl)-thiophen-2-yl, 5-(3-dimethylaminomethyl-phenyl)-thiophen-2-yl, 6-(4-dimethylaminomethyl-phenyl)-pyridin-3-yl, 6-(3-dimethylaminomethyl-phenyl)-pyridin-3-yl, 6-[4-(2-pyrrolidin-1-yl-ethoxy)-phenyl]-pyridin-3-yl, 6-[3-(2-pyrrolidin-1-yl-ethoxy)-phenyl]-pyridin-3-yl, 5-(3-aminomethyl-phenyl)-thiophen-2-yl, 5-[3-(acetylamino)-methyl-phenyl]-thiophen-2-yl, 5-[3-(methylsulphonylamino)-methyl-phenyl]-thiophen-2-yl, 5-(4-dimethylsulphamoyl-phenyl)-thiophen-2-yl, 5-(4-aminomethyl-phenyl)-thiophen-2-yl, 5-[4-(acetylamino)-methyl-phenyl]-thiophen-2-yl, 5-[4-(methylsulphonylamino)-methyl-phenyl]-thiophen-2-yl, 5-(3-dimethylsulphamoyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl, 4-(6-amino-pyridin-3-yl)-phenyl, 3-(6-amino-pyridin-3-yl)-phenyl, 4-(6-methoxy-pyridin-3-yl)-phenyl, 3-(6-methoxy-pyridin-3-yl)-phenyl,
3-(1-methyl-pyrazol-4-yl)-phenyl, 4-(1-methyl-pyrazol-4-yl)-phenyl, 4-(3,5-dimethyl-isoxazol-4-yl)-phenyl, 3-(3,5-dimethyl-isoxazol-4-yl)-phenyl, 4-(1-methyl-indol-5-yl)-phenyl, 3-(1-methyl-indol-5-yl)-phenyl,
4-{1-(2-morpholin-4-yl-ethyl)-[1,2,3]triazol-4-yl}-phenyl, 4-{1-(2-piperidin-1-yl-ethyl)-[1,2,3]triazol-4-yl}-phenyl, 3-{1-(2-morpholin-4-yl-ethyl)-[1,2,3]triazol-4-yl}-phenyl, 3-{1-(2-piperidin-1-yl-ethyl)-[1,2,3]triazol-4-yl}-phenyl, 4-(2,3-dihydrobenzofuran-5-yl)-phenyl, and 4-(benzo[1,3]dioxol-5-yl)-phenyl, 3-(2,3-dihydrobenzofuran-5-yl)-phenyl, and 3-(benzo[1,3]dioxol-5-yl)-phenyl,
R7 is 2-aminophenyl,
and the salts of these compounds.

Compounds according to aspect B of the present invention to be more emphasized are those compounds of formula I in which
R1, R2, R3, R4 and R5 are independently hydrogen or 1-4C-alkyl,
R6 is -T1-Q1, in which T1 is a bond; either
Q1 is substituted by R61 and/or R62, and is Aa1, Hh1, Ha1, Ha2, Ha3 or Ah1,
Or Q1 is unsubstituted, and is Ha2 or Ha3,
in which
R61 is 1-4C-alkyl, 1-4C-alkoxy, halogen, hydroxy-1-4C-alkyl, 1-4C-alkylsulphonylamino, tolylsulphonylamino, phenylsulphonylamino, 1-4C-alkylcarbonylamino, di-1-4C-alkylaminosulphonyl, -T2-N(R611)R612, or -U-T3-N(R613)R614, in which
T2 is a bond or 1-4C-alkylene,
R611 is hydrogen, 1-4C-alkyl, 3-7C-cycloalkyl or 1-4C-alkoxy-2-4C-alkyl,
R612 is hydrogen or 1-4C-alkyl,
or R611 and R612 together and with inclusion of the nitrogen atom, to which they are bonded, form a heterocyclic ring Het1, in which Het1 is morpholino, piperidino, pyrrolidino, piperazino or 4N-(1-4C-alkyl)-piperazino,
U is -O- (oxygen) or —C(O)NH—,
T3 is 2-4C-alkylene,
R613 is hydrogen, 1-4C-alkyl, 3-7C-cycloalkyl or 1-4C-alkoxy-2-4C-alkyl,
R614 is hydrogen or 1-4C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
R62 is 1-4C-alkyl,
Aa1 is biphenyl,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
R7 is hydroxyl, or 2-aminophenyl,
and the salts of these compounds.

Compounds according to aspect B of the present invention to be more emphasized are those compounds of formula I in which
R1, R2, R3, R4 and R5 are hydrogen,
R6 is -T1-Q1, in which T1 is a bond;
Q1 is substituted by R61 and/or R62 on the terminal ring, and is Aa1, Hh1, Ha1, Ha2 or Ah1,
in which
R61 is 1-2C-alkyl, 1-2C-alkoxy, halogen, hydroxy-1-2C-alkyl, 1-2C-alkylsulphonylamino, 1-2C-alkylcarbonylamino, di-1-2C-alkylaminosulphonyl, -T2-N(R611)R612, or-U-T3-N(R613)R614, in which
T2 is a bond or straight chain 1-4C-alkylene,
R611 is hydrogen, 1-2C-alkyl, 3-5C-cycloalkyl or 1-2C-alkoxy-2-3C-alkyl,
R612 is hydrogen or 1-2C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-2C-alkyl)-piperazino,
U is —O— (oxygen) or —C(O)NH—,
T3 is straight chain 2-4C-alkylene,
R613 is hydrogen, 1-2C-alkyl, 3-5C-cycloalkyl or 1-2C-alkoxy-2-3C-alkyl,
R614 is hydrogen or 1-2C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
R62 is 1-2C-alkyl,
Aa1 is 1,1′-biphenyl-3-yl or 1,1′-biphenyl-4-yl,
Hh1 is a bisheteroaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a thiophenyl group, whereby said heteroaryl and thiophenyl groups are linked together via a single bond, and whereby Hh1 is bonded via said thiophenyl moiety to the to the parent molecular group,
Ah1 is phenyl-thiophenyl,
Ha1 is a 3-(heteroaryl)-phenyl or 4-(heteroaryl)-phenyl radical each made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a 3-(heteroaryl)-phenyl or 4-(heteroaryl)-phenyl radical each made up of a heteroaryl group selected from a group consisting of fused bicyclic 9- or 10-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
R7 is hydroxyl, or 2-aminophenyl,
and the salts of these compounds.

Compounds according to aspect B of the present invention to be more emphasized are those compounds of formula I in which
R1, R2, R3, R4 and R5 are hydrogen,
R6 is -T1-Q1, in which T1 is a bond;
Q1 is substituted by R61 and/or R62 on the terminal ring, and is Aa1, Hh1, Ha1, Ha2 or Ah1,
in which
R61 is 1-2C-alkyl, 1-2C-alkoxy, hydroxyl, trifluoromethyl, halogen, hydroxy-1-2C-alkyl, 1-2C-alkylsulphonylamino, 1-2C-alkylcarbonylamino, di-1-2C-alkylaminosulphonyl, -T2-N(R611)R612, or -U-T3-N(R613)R614, in which
T2 is a bond or straight chain 1-4C-alkylene,
R611 is hydrogen, 1-2C-alkyl, 3-5C-cycloalkyl or 1-2C-alkoxy-2-3C-alkyl,
R612 is hydrogen or 1-2C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-2C-alkyl)-piperazino,
U is —O— (oxygen) or —C(O)NH—,
T3 is straight chain 2-4C-alkylene,
R613 is hydrogen, 1-2C-alkyl, 3-5C-cycloalkyl or 1-2C-alkoxy-2-3C-alkyl,
R614 is hydrogen or 1-2C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
R62 is 1-2C-alkyl,
Aa1 is 1,1′-biphenyl-3-yl or 1,1′-biphenyl-4-yl,
Hh1 is a bisheteroaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a thiophenyl group, whereby said heteroaryl and thiophenyl groups are linked together via a single bond, and whereby Hh1 is bonded via said thiophenyl moiety to the to the parent molecular group,
Ah1 is phenyl-thiophenyl or phenyl-pyridinyl,
Ha1 is a 3-(heteroaryl)-phenyl or 4-(heteroaryl)-phenyl radical each made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a 3-(heteroaryl)-phenyl or 4-(heteroaryl)-phenyl radical each made up of a heteroaryl group selected from a group consisting of fused bicyclic 9- or 10-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
R7 is hydroxyl, or 2-aminophenyl,
and the salts of these compounds.

Compounds according to aspect B of the present invention to be more emphasized are those compounds of formula I in which
R1, R2, R3, R4 and R5 are hydrogen,
R6 is -T1-Q1, in which T1 is a bond;
Q1 is substituted by R61 on the terminal ring, and is Aa1 or Ah1, in which
Aa1 is 1,1′-biphenyl-3-yl or 1,1′-biphenyl-4-yl,
Ah1 is phenyl-thiophenyl,
R61 is methoxy, hydroxymethyl, methylsulphonylamino, methylcarbonylamino, dimethylaminosulphonyl, -T2-N(R611)R612, or -U-T3-N(R613)R614, in which
T2 is a bond, methylene, dimethylene or trimethylene,
R611 is hydrogen, methyl, cyclopropyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4N-methyl-piperazino,
U is —O— (oxygen) or —C(O)NH—,
T3 is dimethylene or trimethylene,
R613 is hydrogen, methyl, cyclopropyl or 2-methoxyethyl,
R614 is hydrogen or methyl,
or R613 and R614 together and with inclusion of the nitrogen atom, to which they are bonded, form a heterocyclic ring Het2, in which
Het2 is morpholino, piperidino, pyrrolidino, piperazino or 4N-methyl-piperazino, or
Q1 is substituted by R61 on the terminal ring, and is Hh1 or Ha1, in which
Hh1 is pyridinyl-thiophenyl,
Ha1 is 3-(pyridinyl)-phenyl or 4-(pyridinyl)-phenyl,
R61 is methoxy, or -T2-N(R611)R612, in which
T2 is a bond, methylene, dimethylene or trimethylene,
R611 is hydrogen, methyl, cyclopropyl or 2-methoxyethyl,
R612 is hydrogen or methyl,
or R611 and R612 together and with inclusion of the nitrogen atom, to which they are bonded, form a heterocyclic ring Het1, in which Het1 is morpholino, piperidino, pyrrolidino, piperazino or 4N-methyl-piperazino, or
Q1 is 3-(1N-methyl-pyrazolyl)-phenyl, 4-(1N-methyl-pyrazolyl)-phenyl, 3-(1 N-methyl-indolyl)-phenyl or 4-(1 N-methyl-indolyl)-phenyl,
R7 is hydroxyl, or 2-aminophenyl,
and the salts of these compounds.

Yet compounds according to aspect B of the present invention in more particular worthy to be mentioned are those compounds of formula I in which
R1, R2, R3, R4 and R5 are all hydrogen,
R6 is -T1-Q1, in which T1 is a bond,
Q is (1N-methyl-pyrazolyl)-thiophenyl, 3-(dimethyl-isoxazolyl)-phenyl or 4-(dimethyl-isoxazolyl)-phenyl,
and the salts of these compounds.

In another embodiment, still yet compounds according to aspect B of the present invention in more particular worthy to be mentioned are those compounds of formula I in which
R1, R2, R3, R4 and R5 are all hydrogen,
R6 is -T1-Q1, in which T1 is a bond,
Q1 is substituted by R61 on the terminal ring, and is Aa1 or Ah1, in which
Aa1 is 1,1′-biphenyl-3-yl or 1,1′-biphenyl-4-yl,
Ah1 is phenyl-thiophenyl or phenyl-pyridinyl,
R61 is methoxy, hydroxyl, trifluoromethyl, hydroxymethyl, methylsulphonylamino, methylcarbonylamino, dimethylaminosulphonyl, -T2-N(R611)R612, or -U-T3-N(R613)R614, in which
T2 is a bond, methylene, dimethylene or trimethylene,
R611 is hydrogen, methyl, cyclopropyl, cyclopentyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4N-methyl-piperazino,
U is —O— (oxygen) or —C(O)NH—,
T3 is dimethylene or trimethylene,
R613 is hydrogen, methyl, cyclopropyl, cyclopentyl or 2-methoxyethyl,
R614 is hydrogen or methyl,
or R613 and R614 together and with inclusion of the nitrogen atom, to which they are bonded, form a heterocyclic ring Het2, in which
Het2 is morpholino, piperidino, pyrrolidino, piperazino or 4N-methyl-piperazino, or
Q1 is substituted by R61 on the terminal ring, and is Hh1 or Ha1, in which
Hh1 is pyridinyl-thiophenyl or bipyridyl,
Ha1 is 3-(pyridinyl)-phenyl or 4-(pyridinyl)-phenyl,
R61 is methoxy, or -T2-N(R611)R612, in which
T2 is a bond, methylene, dimethylene or trimethylene,
R611 is hydrogen, methyl, cyclopropyl, cyclopentyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4N-methyl-piperazino, or
Q1 is 3-(1N-methyl-pyrazolyl)-phenyl, 4-(1N-methyl-pyrazolyl)-phenyl, (1N-methyl-pyrazolyl)-thiophenyl, (1N-methyl-pyrazolyl)-pyridinyl, 3-(methyl-thiazolyl)-phenyl, 4-(methyl-thiazolyl)-phenyl, (methyl-thiazolyl)-thiophenyl, (methyl-thiazolyl)-pyridinyl, 3-(dimethyl-isoxazolyl)-phenyl, 4-(dimethyl-isoxazolyl)-phenyl, 3-(1 N-methyl-indolyl)-phenyl or 4-(1 N-methyl-indolyl)-phenyl,
R7 is hydroxyl, or 2-aminophenyl,
and the salts of these compounds.

Compounds according to aspect B of the present invention to be emphasized are those compounds of formula I in which
R1, R2, R3, R4 and R5 are all hydrogen,
R6 is -T1-Q1, in which T1 is a bond,
Q1 is substituted by R61 on the terminal ring, and is Aa1 or Ah1, in which
Aa1 is 1,1′-biphenyl-3-yl or 1,1′-biphenyl-4-yl,
Ah1 is phenyl-thiophenyl,
R61 is hydroxymethyl, methylsulphonylamino, methylcarbonylamino, dimethylaminosulphonyl, -T2-N(R611)R612, or -U-T3-N(R613)R614, in which
T2 is methylene, dimethylene or trimethylene,
R611 is methyl, cyclopropyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino,
U is —O— (oxygen) or —C(O)NH—,
T3 is dimethylene or trimethylene,
R613 and R614 are methyl,
or R613 and R614 together and with inclusion of the nitrogen atom, to which they are bonded, form a heterocyclic ring Het2, in which Het2 is morpholino, piperidino, pyrrolidino or 4N-methyl-piperazino, or
Q1 is substituted by R61 on the terminal ring, and is Hh1 or Ha1, in which
Hh1 is pyridinyl-thiophenyl,
Ha1 is 3-(pyridinyl)-phenyl or 4-(pyridinyl)-phenyl,
R61 is methoxy, or -T2-N(R611)R612, in which
T2 is a bond,
R611 and R612 are independently is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino, or
Q1 is 3-(1N-methyl-pyrazolyl)-phenyl, 4-(1N-methyl-pyrazolyl)-phenyl, 3-(1 N-methyl-indolyl)-phenyl or 4-(1 N-methyl-indolyl)-phenyl,
R7 is hydroxyl, or 2-aminophenyl,
and the salts of these compounds.

Yet compounds according to aspect B of the present invention to be emphasized are those compounds of formula I in which
R1, R2, R3, R4 and R5 are all hydrogen,
R6 is -T1-Q1, in which T1 is a bond,
Q is (1N-methyl-pyrazol-4-yl)-thiophenyl, 3-(dimethyl-isoxazolyl)-phenyl or 4-(dimethyl-isoxazolyl)-phenyl,
and the salts of these compounds.

In another embodiment, still yet compounds according to aspect B of the present invention to be emphasized are those compounds of formula I in which
R1, R2, R3, R4 and R5 are all hydrogen,
R6 is -T1-Q1, in which T1 is a bond,
Q1 is 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is methoxy, hydroxyl, trifluoromethyl, hydroxymethyl, methylsulphonylamino, methylcarbonylamino, dimethylaminosulphonyl, -T2-N(R611)R612, or -U-T3-N(R613)R614, in which
T2 is a bond, methylene, dimethylene or trimethylene,
R611 is hydrogen, methyl, cyclopropyl, cyclopentyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino, either
U is —O— (oxygen),
T3 is dimethylene or trimethylene,
R613 and R614 together and with inclusion of the nitrogen atom, to which they are bonded, form a heterocyclic ring Het2, in which
Het2 is morpholino, piperidino, pyrrolidino or 4N-methyl-piperazino, or
U is —C(O)NH—,
T3 is dimethylene or trimethylene,
R613 and R614 are methyl, or
Q1 is 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl, in which
R61 is methoxy, hydroxyl, trifluoromethyl, hydroxymethyl, methylsulphonylamino, methylcarbonylamino, dimethylaminosulphonyl, -T2-N(R611)R612, or -U-T3-N(R613)R614, in which T2 is a bond, methylene, dimethylene or trimethylene, either
R611 is methyl, cyclopropyl, cyclopentyl or 2-methoxyethyl,
R612 is hydrogen,
or R611 and R612 are hydrogen,
or R611 and R612 are methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino, either
U is —O— (oxygen),
T3 is dimethylene or trimethylene,
R613 and R614 together and with inclusion of the nitrogen atom, to which they are bonded, form a heterocyclic ring Het2, in which
Het2 is morpholino, piperidino, pyrrolidino or 4N-methyl-piperazino, or
Q1 is 5-[2-(R61)-pyridin-4-yl]-thiophen-2-yl or 5-[6-(R61)-pyridin-3-yl]-thiophen-2-yl, in which
R61 is amino, methoxy, dimethylamino, or -T2-N(R611)R612, in which
T2 is a bond,
R611 and R612 together and with inclusion of the nitrogen atom, to which they are bonded, form a heterocyclic ring Het1, in which
Het1 is morpholino, piperidino, pyrrolidino or 4N-methyl-piperazino, or
Q1 is 2-[2-(R61)-pyridin-4-yl]-pyridin-4-yl, 2-[6-(R61)-pyridin-3-yl]-pyridin-4-yl, 3-[2-(R61)-pyridin-4-yl]-pyridin-6-yl or 3-[6-(R61)-pyridin-3-yl]-pyridin-6-yl, in which
R61 is amino, methoxy, dimethylamino, or -T2-N(R611)R612, in which
T2 is a bond,
R611 and R612 together and with inclusion of the nitrogen atom, to which they are bonded, form a heterocyclic ring Het1, in which
Het1 is morpholino, piperidino, pyrrolidino or 4N-methyl-piperazino, or
Q1 is 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which
R61 is amino, methoxy, dimethylamino, or -T2-N(R611)R612, in which
T2 is a bond,
R611 and R612 together and with inclusion of the nitrogen atom, to which they are bonded, form a heterocyclic ring Het1, in which
Het1 is morpholino, piperidino, pyrrolidino or 4N-methyl-piperazino, or
Q1 is 3-(1N-methyl-pyrazol-4-yl)-phenyl, 4-(1N-methyl-pyrazol-4-yl)-phenyl, 5-(1N-methyl-pyrazol-4-yl)-thiophen-2-yl, 6-(1N-methyl-pyrazol-4-yl)-pyridin-3-yl, 5-(2-methyl-thiazol-4-yl)-thiophen-2-yl, 3-(3,5-dimethyl-isoxazol-4-yl)-phenyl, 4-(3,5-dimethyl-isoxazol-4-yl)-phenyl, 3-(1N-methyl-indol-5-yl)-phenyl or 4-(1N-methyl-indol-5-yl)-phenyl,
R7 is hydroxyl, or 2-aminophenyl,
and the salts of these compounds.

Compounds according to aspect B of the present invention to be more emphasized are those compounds of formula I in which
R1, R2, R3, R4 and R5 are all hydrogen,
R6 is -T1-Q1, in which T1 is a bond,
Q1 is 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is hydroxymethyl, methylsulphonylamino, methylcarbonylamino, dimethylaminosulphonyl, -T2-N(R611)R612, or -U-T3-N(R613)R614, in which
T2 is methylene, dimethylene or trimethylene,
R611 is methyl, cyclopropyl or 2-methoxyethyl,
R612 is hydrogen or methyl,
or R611 and R612 together and with inclusion of the nitrogen atom, to which they are bonded, form a heterocyclic ring Het1, in which Het1 is morpholino, pyrrolidino or 4N-methyl-piperazino, either
U is —O— (oxygen),
T3 is dimethylene or trimethylene,
R613 and R614 together and with inclusion of the nitrogen atom, to which they are bonded, form a heterocyclic ring Het2, in which
Het2 is morpholino, pyrrolidino or 4N-methyl-piperazino, or
U is —C(O)NH—,
T3 is dimethylene or trimethylene,
R613 and R614 aremethyl, or
Q1 is 5-[3-(R61)-phenyl]-thiophen-2-yl or 5-[4-(R61)-phenyl]-thiophen-2-yl, in which
R61 is -T2-N(R611)R612, or -U-T3-N(R613)R614, in which
T2 is methylene, dimethylene or trimethylene,
R611 and R612 are methyl,
or R611 and R612 together and with inclusion of the nitrogen atom, to which they are bonded, form a heterocyclic ring Het1, in which
Het1 is morpholino, pyrrolidino or 4N-methyl-piperazino,
U is —O— (oxygen),
T3 is dimethylene or trimethylene,
R613 and R614 together and with inclusion of the nitrogen atom, to which they are bonded, form a heterocyclic ring Het2, in which Het2 is morpholino, pyrrolidino or 4N-methyl-piperazino, or
Q1 is 5-[2-(R61)-pyridin-4-yl]-thiophen-2-yl or 5-[6-(R61)-pyridin-3-yl]-thiophen-2-yl, in which
R61 is amino, or -T2-N(R611)R612, in which T2 is a bond,
R611 and R612 together and with inclusion of the nitrogen atom, to which they are bonded, form a heterocyclic ring Het1, in which Het1 is morpholino, pyrrolidino or 4N-methyl-piperazino,
Q1 is 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which
R61 is amino, methoxy, or -T2-N(R611)R612, in which T2 is a bond,
R611 and R612 together and with inclusion of the nitrogen atom, to which they are bonded, form a heterocyclic ring Het1, in which Het1 is morpholino, pyrrolidino or 4N-methyl-piperazino, or
Q1 is 3-(1N-methyl-pyrazol-4-yl)-phenyl, 4-(1N-methyl-pyrazol-4-yl)-phenyl, 3-(1 N-methyl-indol-5-yl)-phenyl or 4-(1 N-methyl-indol-5-yl)-phenyl,
R7 is hydroxyl, or 2-aminophenyl,
and the salts of these compounds.

Yet compounds according to aspect B of the present invention to be more emphasized are those compounds of formula I in which
R1, R2, R3, R4 and R5 are all hydrogen,
R6 is -T1-Q1, in which T1 is a bond,
Q is 5-(1N-methyl-pyrazol-4-yl)-thiophen-2-yl, 3-(3,5-dimethyl-isoxazol-4-yl)-phenyl or 4-(3,5-dimethyl-isoxazol-4-yl)-phenyl,
and the salts of these compounds.

Compounds according to aspect B of the present invention to be in particular emphasized are those compounds of formula I in which
R1, R2, R3, R4 and R5 are all hydrogen,
R6 is -T1-Q1, in which T1 is a bond,
Q1 is any one selected from the group consisting of
3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 4′-(3-morpholin-4-yl-propyl)-biphenyl-3-yl, 4′-(4-methyl-piperazin-1-ylmethyl)-biphenyl-3-yl, 4′-(2-morpholin-4-yl-ethoxy)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, 4′-(2-pyrrolidin-1-yl-ethoxy]-biphenyl-3-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 2′-methylsulphonylamino-biphenyl-4-yl, 3′-methylsulphonylamino-biphenyl-4-yl, 4′-methylsulphonylamino-biphenyl-4-yl, 4′-dimethylsulphamoyl-biphenyl-4-yl, 3′-acetamido-biphenyl-4-yl, 4′-acetamido-biphenyl-4-yl, 4′-(2-methoxy-ethylamino)methylbiphenyl-3-yl, 4′-cyclopropylaminomethyl-biphenyl-3-yl, 3′-hydroxymethyl-biphenyl-4-yl,
5-[2-(4-methyl-piperazin-1-yl)-pyridin-4-yl]-thiophen-2-yl, 5-(1N-methyl-pyrazol-4-yl)-thiophen-2-yl,
5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 5-[4-(2-morpholin-4-yl-ethoxy)-phenyl]-thiophen-2-yl,
5-[4-(3-morpholin-4-yl-propoxy)-phenyl]-thiophen-2-yl, 5-{4-[2-(4-methyl-piperazin-1-yl)-ethoxy]-phenyl}-thiophen-2-yl, 5-(4-dimethylaminomethyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl,
4-[6-amino-pyridin-3-yl]-phenyl, 3-[6-amino-pyridin-3-yl]-phenyl, 4-[6-methoxy-pyridin-3-yl]-phenyl, 3-[6-methoxy-pyridin-3-yl]-phenyl,
3-(1N-methyl-pyrazol-4-yl)-phenyl, 4-(1N-methyl-pyrazol-4-yl)-phenyl, 4-(3,5-dimethyl-isoxazol-4-yl)-phenyl, and 4-(1N-methyl-indol-5-yl)-phenyl,
R7 is hydroxyl, or 2-aminophenyl,
and the salts of these compounds.

In one embodiment, compounds according to aspect B of the present invention to be in more particular emphasized are those compounds of formula I in which
R1, R2, R3, R4 and R5 are all hydrogen,
R6 is -T1-Q1, in which T1 is a bond,
Q1 is any one selected from the group consisting of
4′-(2-morpholin-4-yl-ethyl)-biphenyl-3-yl, 4′-(3-morpholin-4-yl-propoxy)-biphenyl-3-yl,
4′-[2-(4-methyl-piperazin-1-yl)-ethoxy]-biphenyl-3-yl, 4′-dimethylaminomethyl-biphenyl-4-yl,
5-[2-(4-methyl-piperazin-1-yl)-pyridin-4-yl]-thiophen-2-yl, 5-(4-dimethylaminomethyl-phenyl)-thiophen-2-yl,
4-[2-(4-methyl-piperazin-1-yl)-pyridin-4-yl]-phenyl, 3-[2-(4-methyl-piperazin-1-yl)-pyridin-4-yl]-phenyl,
4-[6-amino-pyridin-3-yl]-phenyl, and 4-(1N-methyl-pyrazol-4-yl)-phenyl.
R7 is hydroxyl,
and the salts of these compounds.

In another embodiment, compounds according to aspect B of the present invention to be in more particular emphasized are those compounds of formula I in which
R1, R2, R3, R4 and R5 are all hydrogen,
R6 is -T1-Q1, in which T1 is a bond,
Q1 is any one selected from the group consisting of
4′-(2-morpholin-4-yl-ethyl)-biphenyl-3-yl, 4′-(3-morpholin-4-yl-propoxy)-biphenyl-3-yl,
4′-[2-(4-methyl-piperazin-1-yl)-ethoxy]-biphenyl-3-yl, 4′-dimethylaminomethyl-biphenyl-4-yl,
5-[2-(4-methyl-piperazin-1-yl)-pyridin-4-yl]-thiophen-2-yl, 5-(4-dimethylaminomethyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, and 4-(1N-methyl-pyrazol-4-yl)-phenyl,
R7 is 2-aminophenyl,
and the salts of these compounds.

In a first embodiment of aspect C (embodiment C1) of the present invention, compounds according to aspect C of the present invention more worthy to be mentioned are those compounds of formula I in which
R1, R2, R3, R4, and R5 are independently hydrogen, or 1-4C-alkyl,
R6 is -T1-Q1, in which T1 is a bond, either
Q1 is substituted by R61 and/or R62, and is Aa1, Hh1, Ha1, Ha2, Ha3 or Ah1, or Q1 is unsubstituted, and is Ha2 or Ha3,
in which
R61 is 1-4C-alkyl, 1-4C-alkoxy, halogen, or -T2-N(R611)R612, in which
T2 is a bond or 1-4C-alkylene,
R611 and R612 are independently hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
R62 is 1-4C-alkyl,
Aa1 is biphenyl,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
R7 is hydroxyl, or 2-aminophenyl,
and the salts of these compounds.

In a second embodiment of aspect C (embodiment C2), compounds according to aspect C of the present invention more worthy to be mentioned are those compounds of formula I in which
R1, R2, R3, R4 and R5 are independently hydrogen, or 1-4C-alkyl,
R6 is -T1-Q1, in which T1 is a bond, either
Q1 is substituted by R61, and is Aa1, Ha1, Ha2 or Ha3,
Or Q1 is unsubstituted, and is Ha2 or Ha3,
in which
R61 is 1-4C-alkyl, 1-4C-alkoxy, halogen, or -T2-N(R611)R612, in which
T2 is a bond or 1-4C-alkylene,
R611 and R612 are independently hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
Aa1 is biphenyl,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
R7 is hydroxyl, or 2-aminophenyl,
and the salts of these compounds.

Compounds according to embodiment C1 of aspect C of the present invention in particular worthy to be mentioned are those compounds of formula I in which
R1, R2, R3, R4 and R5 are hydrogen,
R6 is -T1-Q1, in which T1 is a bond, either
Q1 is substituted by R61 on the terminal ring, and is Aa1, Hh1, Ha1 or Ah1,
or Q1 is [1N-(1-4C-alkyl)-indolyl]-phenyl, [1N-(1-4C-alkyl)-pyrazolyl]-phenyl, [1N-(1-4C-alkyl)-imidazolyl]-phenyl, [1N-(1-4C-alkyl)-triazolyl]-phenyl, [1N-(1-4C-alkyl)-tetrazolyl]-phenyl, [1N-(1-4C-alkyl)-benzimidazolyl]-phenyl, [1N-(1-4C-alkyl)-benztriazolyl]-phenyl, or [1N-(1-4C-alkyl)-indazol]-phenyl,
or Q1 is [1N-(1-4C-alkyl)-indolyl]-thiophenyl, [1N-(1-4C-alkyl)-pyrazolyl]-thiophenyl, [1N-(1-4C-alkyl)-imidazolyl]-thiophenyl, [1N-(1-4C-alkyl)-triazolyl]-thiophenyl, [1N-(1-4C-alkyl)-tetrazolyl]-thiophenyl, [1N-(1-4C-alkyl)-benzimidazolyl]-thiophenyl, [1N-(1-4C-alkyl)-benztriazolyl]-thiophenyl, or [1N-(1-4C-alkyl)-indazol]-thiophenyl,
or Q1 is [mono- or di-(1-4C-alkyl)-isoxazolyl]-phenyl, or [mono- or di-(1-4C-alkyl)-isoxazolyl]-thiophenyl,
in which
R61 is 1-4C-alkyl, 1-4C-alkoxy, halogen, or -T2-N(R611)R612, in which
T2 is a bond or 1-4C-alkylene,
R611 is hydrogen or 1-4C-alkyl, R612 is hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
Aa1 is 1,1′-biphenyl-4-yl or 1,1′-biphenyl-3-yl,
Hh1 is pyridinyl-thiophenyl,
Ha1 is 3-(pyridinyl)-phenyl or 4-(pyridinyl)-phenyl,
Ah1 is phenyl-thiophenyl,
R7 is hydroxyl, or 2-aminophenyl,
and the salts of these compounds.

Compounds according to embodiment C2 of aspect C of the present invention in particular worthy to be mentioned are those compounds of formula I in which
R1, R2, R3, R4 and R5 are hydrogen,
R6 is -T1-Q1, in which T1 is a bond, either
Q1 is substituted by R61 on the terminal ring, and is Aa1 or Ha1,
or Q1 is [1N-(1-4C-alkyl)-indolyl]-phenyl, [1N-(1-4C-alkyl)-pyrazolyl]-phenyl, [1N-(1-4C-alkyl)-imidazolyl]-phenyl, [1N-(1-4C-alkyl)-triazolyl]-phenyl, [1N-(1-4C-alkyl)-tetrazolyl]-phenyl, [1N-(1-4C-alkyl)-benzimidazolyl]-phenyl, [1N-(1-4C-alkyl)-benztriazolyl]-phenyl, or [1N-(1-4C-alkyl)-indazol]-phenyl,
in which
R61 is 1-4C-alkyl, 1-4C-alkoxy, halogen, or -T2-N(R611)R612, in which
T2 is a bond or 1-4C-alkylene,
R611 is hydrogen or 1-4C-alkyl,
R612 is hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
Aa1 is 1,1′-biphenyl-4-yl or 1,1′-biphenyl-3-yl,
Ha1 is 3-(pyridinyl)-phenyl or 4-(pyridinyl)-phenyl,
R7 is hydroxyl, or 2-aminophenyl,
and the salts of these compounds.

Compounds according to embodiment C1 of aspect C of the present invention in more particular worthy to be mentioned are those compounds of formula I in which
R1, R2, R3, R4 and R5 are hydrogen,
R6 is -T1-Q1, in which T1 is a bond, either
Q1 is substituted by R61 on the pyridine ring, and is 3-(pyridinyl)-phenyl or 4-(pyridinyl)-phenyl,
or Q1 is 2′-(R61)-1,1′-biphenyl-4-yl, 3′-(R61)-1,1′-biphenyl-4-yl, 4′-(R61)-1,1′-biphenyl-4-yl, 2′-(R61)-1,1′-biphenyl-3-yl, 3′-(R61)-1,1′-biphenyl-3-yl or 4′-(R61)-1,1′-biphenyl-3-yl,
or Q1 is substituted by R61 on the pyridine ring, and is pyridinyl-thiophenyl,
or Q1 is substituted by R61 on the phenyl ring, and is phenyl-thiophenyl,
or Q1 is 3-[1N-methyl-indolyl]-phenyl, 4-[1N-methyl-indolyl]-phenyl, 3-[1N-methyl-pyrazolyl]-phenyl or 4-[1N-methyl-pyrazolyl]-phenyl,
or Q1 is [1N-methyl-pyrazolyl]-thiophenyl,
or Q1 is 3-[dimethyl-isoxazolyl]-phenyl or 4-[dimethyl-isoxazolyl]-phenyl,
in which
R61 is 1-2C-alkoxy, amino, or -T2-N(R611)R612, in which
T2 is a bond, methylene, dimethylene or trimethylene,
R611 is 1-2C-alkyl,
R612 is 1-2C-alkyl,
or R611 and R612 together and with inclusion of the nitrogen atom, to which they are bonded, form a heterocyclic ring Het1, in which
Het1 is morpholino, pyrrolidino or 4N-methyl-piperazino,
R7 is hydroxyl, or 2-aminophenyl,
and the salts of these compounds.

Compounds according to embodiment C2 of aspect C of the present invention in more particular worthy to be mentioned are those compounds of formula I in which
R1, R2, R3, R4 and R5 are hydrogen,
R6 is -T1-Q1, in which T1 is a bond, either
Q1 is substituted by R61 on the pyridine ring, and is 3-(pyridinyl)-phenyl or 4-(pyridinyl)-phenyl,
or Q1 is 3′-(R61)-1,1′-biphenyl-4-yl or 4′-(R61)-1,1′-biphenyl-4-yl,
or Q1 is 3-[1N-methyl-indolyl]-phenyl, 4-[1N-methyl-indolyl]-phenyl, 3-[1N-methyl-pyrazolyl]-phenyl or 4-[1N-methyl-pyrazolyl]-phenyl,
in which
R61 is 1-2C-alkoxy, amino, or -T2-N(R611)R612, in which
T2 is a bond or 1-2C-alkylene,
R611 and R612 are 1-2C-alkyl,
or R611 and R612 together and with inclusion of the nitrogen atom, to which they are bonded, form a heterocyclic ring Het1, in which
Het1 is morpholino,
R7 is hydroxyl, or 2-aminophenyl,
and the salts of these compounds.

Compounds according to embodiment C1 of aspect C of the present invention to be emphasized are, in one embodiment, those compounds of formula in which
R1, R2, R3, R4 and R5 are hydrogen,
R6 is -T1-Q1, in which T1 is a bond, either
Q1 is 3-(6-amino-pyridin-3-yl)-phenyl, 4-(6-amino-pyridin-3-yl)-phenyl, 3-(6-methoxy-pyridin-3-yl)-phenyl or 4-(6-methoxy-pyridin-3-yl)-phenyl,
or Q1 is 3-[2-(4-methyl-piperazin-1-yl)-pyridin-4-yl)]-phenyl or 4-[2-(4-methyl-piperazin-1-yl)-pyridin-4-yl)]-phenyl,
or Q1 is 3′-(R61)-1,1′-biphenyl-4-yl or 4′-(R61)-1,1′-biphenyl-4-yl,
or Q1 is 5-[2-(4-methyl-piperazin-1-yl)-pyridin-4-yl)]-thiophen-2-yl,
or Q1 is 5-[4-(R61)-phenyl]-thiophen-2-yl or 5-[3-(R61)-phenyl]-thiophen-2-yl,
or Q1 is 3-(1N-methyl-indol-5-yl)-phenyl, 4-(1N-methyl-indol-5-yl)-phenyl, 3-(1N-methyl-pyrazol-4-yl)-phenyl or 4-(1N-methyl-pyrazol-4-yl)-phenyl,
or Q1 is 5-(1N-methyl-pyrazol-4-yl)-thiophen-2-yl,
or Q1 is 3-(3,5-dimethyl-isoxazol-4-yl)-phenyl or 4-(3,5-dimethyl-isoxazol-4-yl)-phenyl,
in which
R61 is -T2-N(R61)R612, in which
T2 is methylene, dimethylene or trimethylene, either
R611 and R612 are both methyl, or
R611 and R612 together and with inclusion of the nitrogen atom, to which they are bonded, form a heterocyclic ring Het1, in which
Het1 is morpholino or 4N-methyl-piperazino,
R7 is hydroxyl,
and the salts of these compounds.

Compounds according to embodiment C1 of aspect C of the present invention to be emphasized are, in another embodiment, those compounds of formula I in which
R1, R2, R3, R4 and R5 are hydrogen,
R6 is -T1-Q1, in which T1 is a bond, either
Q1 is 3-(6-amino-pyridin-3-yl)-phenyl, 4-(6-amino-pyridin-3-yl)-phenyl, 3-(6-methoxy-pyridin-3-yl)-phenyl or 4-(6-methoxy-pyridin-3-yl)-phenyl,
or Q1 is 3-[2-(4-methyl-piperazin-1-yl)-pyridin-4-yl)]-phenyl or 4-[2-(4-methyl-piperazin-1-yl)-pyridin-4-yl)]-phenyl,
or Q1 is 3′-(R61)-1,1′-biphenyl-4-yl or 4′-(R61)-1,1′-biphenyl-4-yl,
or Q1 is 5-[2-(4-methyl-piperazin-1-yl)-pyridin-4-yl)]-thiophen-2-yl,
or Q1 is 5-[4-(R61)-phenyl]-thiophen-2-yl or 5-[3-(R61)-phenyl]-thiophen-2-yl,
or Q1 is 3-(1N-methyl-indol-5-yl)-phenyl, 4-(1N-methyl-indol-5-yl)-phenyl, 3-(1N-methyl-pyrazol-4-yl)-phenyl or 4-(1N-methyl-pyrazol-4-yl)-phenyl,
or Q1 is 5-(1N-methyl-pyrazol-4-yl)-thiophen-2-yl,
or Q1 is 3-(3,5-dimethyl-isoxazol-4-yl)-phenyl or 4-(3,5-dimethyl-isoxazol-4-yl)-phenyl,
in which
R61 is -T2-N(R611)R612, in which T2 is methylene, dimethylene or trimethylene, either
R611 and R612 are both methyl, or
R611 and R612 together and with inclusion of the nitrogen atom, to which they are bonded, form a heterocyclic ring Het1, in which
Het1 is morpholino or 4N-methyl-piperazino,
R7 is 2-aminophenyl,
and the salts of these compounds.

Compounds according to embodiment C2 of aspect C of the present invention to be emphasized are those compounds of formula I in which
R1, R2, R3, R4 and R5 are hydrogen,
R6 is -T1-Q1, in which T1 is a bond, either
Q1 is 3-(6-amino-pyridin-3-yl)-phenyl, 4-(6-amino-pyridin-3-yl)-phenyl, 3-(6-methoxy-pyridin-3-yl)-phenyl or 4-(6-methoxy-pyridin-3-yl)-phenyl,
or Q1 is 3′-(R61)-1,1′-biphenyl-4-yl or 4′-(R61)-1,1′-biphenyl-4-yl,
or Q1 is 3-(1N-methyl-indol-5-yl)-phenyl, 4-(1N-methyl-indol-5-yl)-phenyl, 3-(1N-methyl-pyrazol-4-yl)-phenyl or 4-(1N-methyl-pyrazol-4-yl)-phenyl,
in which
R61 is -T2-N(R611)R612, in which T2 is 1-2C-alkylene,
R611 and R612 together and with inclusion of the nitrogen atom, to which they are bonded, form a heterocyclic ring Het1, in which Het1 is morpholino,
R7 is hydroxyl, or 2-aminophenyl,
and the salts of these compounds.

Compounds according to embodiment C1 of aspect C of the present invention to be more emphasized are, in one embodiment, those compounds of formula I in which
R1, R2, R3, R4 and R5 are hydrogen,
R6 is -T1-Q1, in which T1 is a bond,
Q1 is any one selected from 3-(6-amino-pyridin-3-yl)-phenyl, 4-(6-amino-pyridin-3-yl)-phenyl, 3-(6-methoxy-pyridin-3-yl)-phenyl, 4-(6-methoxy-pyridin-3-yl)-phenyl, 3-[2-(4-methyl-piperazin-1-yl)-pyridin-4-yl)]-phenyl, 4-[2-(4-methyl-piperazin-1-yl)-pyridin-4-yl)]-phenyl, 3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 4′-(3-morpholin-4-yl-propyl)-biphenyl-3-yl, 4′-(4-methyl-piperazin-1-yl-methyl)-biphenyl-3-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 5-[2-(4-methyl-piperazin-1-yl)-pyridin-4-yl)]-thiophen-2-yl, 5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 4-(1N-methyl-indol-5-yl)-phenyl, 3-(1N-methyl-pyrazol-4-yl)-phenyl, 4-(1N-methyl-pyrazol-4-yl)-phenyl, 5-(4-dimethylaminomethyl-phenyl)-thiophen-2-yl, 5-(1N-methyl-pyrazol-4-yl)-thiophen-2-yl, and 4-(3,5-dimethyl-isoxazol-4-yl)-phenyl, R7 is hydroxyl,
and the salts of these compounds.

Compounds according to embodiment C1 of aspect C of the present invention to be more emphasized are, in another embodiment, those compounds of formula I in which
R1, R2, R3, R4 and R5 are hydrogen,
R6 is -T1-Q1, in which T1 is a bond,
Q1 is any one selected from 3-(6-amino-pyridin-3-yl)-phenyl, 4-(6-amino-pyridin-3-yl)-phenyl, 3-(6-methoxy-pyridin-3-yl)-phenyl, 4-(6-methoxy-pyridin-3-yl)-phenyl, 3-[2-(4-methyl-piperazin-1-yl)-pyridin-4-yl)]-phenyl, 4-[2-(4-methyl-piperazin-1-yl)-pyridin-4-yl)]-phenyl, 3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 4′-(3-morpholin-4-yl-propyl)-biphenyl-3-yl, 4′-(4-methyl-piperazin-1-yl-methyl)-biphenyl-3-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 5-[2-(4-methyl-piperazin-1-yl)-pyridin-4-yl)]-thiophen-2-yl, 5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 4-(1N-methyl-indol-5-yl)-phenyl, 3-(1N-methyl-pyrazol-4-yl)-phenyl, 4-(1N-methyl-pyrazol-4-yl)-phenyl, 5-(4-dimethylaminomethyl-phenyl)-thiophen-2-yl, 5-(1N-methyl-pyrazol-4-yl)-thiophen-2-yl, and 4-(3,5-dimethyl-isoxazol-4-yl)-phenyl,
R7 is 2-aminophenyl,
and the salts of these compounds.

Compounds according to embodiment C1 of aspect C of the present invention to be in particular emphasized are, in one embodiment, those compounds of formula I in which
R1, R2, R3, R4 and R5 are hydrogen,
R6 is -T1-Q1, in which T1 is a bond, Q1 is any one selected from 4-(6-amino-pyridin-3-yl)-phenyl, 4-[2-(4-methyl-piperazin-1-yl)-pyridin-4-yl)]-phenyl, 3-[2-(4-methyl-piperazin-1-yl)-pyridin-4-yl)]-phenyl, 4′-(2-morpholin-4-yl-ethyl)-biphenyl-3-yl, 4′-dimethylaminomethyl-biphenyl-4-yl, 5-[2-(4-methyl-piperazin-1-yl)-pyridin-4-yl)]-thiophen-2-yl, 4-(1N-methyl-pyrazol-4-yl)-phenyl, and 5-(4-dimethylaminomethyl-phenyl)-thiophen-2-yl,
R7 is hydroxyl,
and the salts of these compounds.

Compounds according to embodiment C1 of aspect C of the present invention to be in particular emphasized are, in another embodiment, those compounds of formula I in which
R1, R2, R3, R4 and R5 are hydrogen,
R6 is -T1-Q1, in which T1 is a bond,
Q1 is any one selected from 4-(6-amino-pyridin-3-yl)-phenyl, 4-[2-(4-methyl-piperazin-1-yl)-pyridin-4-yl)]-phenyl, 3-[2-(4-methyl-piperazin-1-yl)-pyridin-4-yl)]-phenyl, 4′-(2-morpholin-4-yl-ethyl)-biphenyl-3-yl, 4′-dimethylaminomethyl-biphenyl-4-yl, 5-[2-(4-methyl-piperazin-1-yl)-pyridin-4-yl)]-thiophen-2-yl, 4-(1N-methyl-pyrazol-4-yl)-phenyl, and 5-(4-dimethylaminomethyl-phenyl)-thiophen-2-yl,
R7 is 2-aminophenyl,
and the salts of these compounds.

A special interest in the compounds according to the present invention refers to those compounds of this invention which are included -within the scope of this invention- by one or, when possible, a combination of more of the following embodiments:

An embodiment of the compounds according to the present invention relates to those compounds of formula I, in which R1, R2, R3, R4 and R5 are all hydrogen.

A further embodiment of the compounds according to the present invention relates to those compounds of formula I, in which R7 is hydroxyl. A further embodiment of the compounds according to the present invention relates to those compounds of formula I, in which R7 is 2-aminophenyl. A further embodiment of the compounds according to the present invention relates to those compounds of formula I, in which R7 is aminopyridyl. A further embodiment of the compounds according to the present invention relates to those compounds of formula I, in which R7 is Cyc1, whereby in a subembodiment thereof Cyc1 is 2-phenyl.

A further embodiment of the compounds according to the present invention relates to those compounds of formula I, in which T1 is a bond.

A further embodiment of the compounds according to the present invention relates to those compounds of formula I, in which R6 is substituted by R61, and is Aa1, Ha1 or Ha2. A further embodiment of the compounds according to the present invention relates to those compounds of formula I, in which R6 is substituted by R61, and is Ah1 or Hh1. A further embodiment of the compounds according to the present invention relates to those compounds of formula I, in which R6 is substituted by R61, and is Ha3. A further embodiment of the compounds according to the present invention relates to those compounds of formula I, in which R6 is 3-(pyridinyl)-phenyl or 4-(pyridinyl)-phenyl, each of which is substituted by R61 on the pyridinyl moiety. A further embodiment of the compounds according to the present invention relates to those compounds of formula I, in which R6 is 3-(pyridin-3-yl)-phenyl, 3-(pyridin-4-yl)-phenyl, 4-(pyridin-3-yl)-phenyl, or 4-(pyridin-4-yl)-phenyl, each of which is substituted by R61 on the pyridinyl moiety. A further embodiment of the compounds according to the present invention relates to those compounds of formula I, in which R6 is 3-(pyridin-3-yl)-phenyl or 4-(pyridin-3-yl)-phenyl, each of which is substituted by R61 on the pyridinyl moiety. A further embodiment of the compounds according to the present invention relates to those compounds of formula I, in which R6 is 3-[6-(R61)-pyridin-3-yl]-phenyl or 4-[6-(R61)-pyridin-3-yl]-phenyl. A further embodiment of the compounds according to the present invention relates to those compounds of formula I, in which R6 is 3-(pyridin-4-yl)-phenyl or 4-(pyridin-4-yl)-phenyl, each of which is substituted by R61 on the pyridinyl moiety. A further embodiment of the compounds according to the present invention relates to those compounds of formula I, in which R6 is 3-[2-(R61)-pyridin-4-yl]-phenyl or 4-[2-(R61)-pyridin-4-yl]-phenyl. A further embodiment of the compounds according to the present invention relates to those compounds of formula I, in which R6 is 1,1′-biphenyl-4-yl or 1,1′-biphenyl-3-yl, each of which is substituted by R61 on the terminal phenyl moiety. A further embodiment of the compounds according to the present invention relates to those compounds of formula I, in which R6 is 3′-(R61)-1,1′-biphenyl-4-yl, 4′-(R61)-1,1′-biphenyl-4-yl, 3′-(R61)-1,1′-biphenyl-3-ylor 4′-(R61)-1,1′-biphenyl-3-yl. A further embodiment of the compounds according to the present invention relates to those compounds of formula I, in which R6 is 3′-(R61)-1,1′-biphenyl-4-yl or 4′-(R61)-1,1′-biphenyl-4-yl. A further embodiment of the compounds according to the present invention relates to those compounds of formula I, in which R6 is 3′-(R61)-1,1′-biphenyl-3-ylor 4′-(R61)-1,1′-biphenyl-3-yl.

A further embodiment of the compounds according to the present invention relates to those compounds of formula I, in which R6 is 4′-(R61)-1,1′-biphenyl-3-ylor 4′-(R61)-1,1′-biphenyl-4-yl.

A further embodiment of the compounds according to the present invention relates to those compounds of formula I, in which R6 is pyridinyl-thiophenyl, which is substituted by R61 on the pyridinyl moiety. A further embodiment of the compounds according to the present invention relates to those compounds of formula I, in which R6 is [2-(R61)-pyridin-4-yl]-thiophenyl, such as e.g. 5-[2-(R61)-pyridin-4-yl]-thiophen-2-yl. A further embodiment of the compounds according to the present invention relates to those compounds of formula I, in which R6 is [6-(R61)-pyridin-3-yl]-thiophenyl, such as e.g. 5-[6-(R61)-pyridin-3-yl]-thiophen-2-yl. A further embodiment of the compounds according to the present invention relates to those compounds of formula I, in which R6 is bipyridyl, which is substituted by R61 on the terminal pyridinyl moiety. A further embodiment of the compounds according to the present invention relates to those compounds of formula I, in which R6 is [2-(R61)-pyridin-4-yl]-pyridinyl, such as e.g. 2-[2-(R61)-pyridin-4-yl]-pyridin-4-yl or 6-[2-(R61)-pyridin-4-yl]-pyridin-3-yl. A further embodiment of the compounds according to the present invention relates to those compounds of formula I, in which R6 is [6-(R61)-pyridin-3-yl]-pyridinyl, such as e.g. 2-[6-(R61)-pyridin-3-yl]-pyridin-4-yl or 6-[6-(R61)-pyridin-3-yl]-pyridin-3-yl. A further embodiment of the compounds according to the present invention relates to those compounds of formula I, in which R6 is phenyl-thiophenyl, which is substituted by R61 on the phenyl moiety. A further embodiment of the compounds according to the present invention relates to those compounds of formula I, in which R6 is [3-(R61)-phenyl]-thiophenyl, such as e.g. 5-[3-(R61)-phenyl]-thiophen-2-yl. A further embodiment of the compounds according to the present invention relates to those compounds of formula I, in which R6 is [4-(R61)-phenyl]-thiophenyl, such as e.g. 5-[4-(R61)-phenyl]-thiophen-2-yl. A further embodiment of the compounds according to the present invention relates to those compounds of formula I, in which R6 is phenyl-pyridinyl, which is substituted by R61 on the phenyl moiety. A further embodiment of the compounds according to the present invention relates to those compounds of formula I, in which R6 is [3-(R61)-phenyl]-pyridinyl, such as e.g. 2-[3-(R61)-phenyl]-pyridin-4-yl or 6-[3-(R61)-phenyl]-pyridin-3-yl. A further embodiment of the compounds according to the present invention relates to those compounds of formula I, in which R6 is [4-(R61)-phenyl]-pyridinyl, such as e.g. 2-[4-(R61)-phenyl]-pyridin-4-yl or 6-[4-(R61)-phenyl]-pyridin-3-yl. A further embodiment of the compounds according to the present invention relates to those compounds of formula I, in which R6 is [1N-(1-4C-alkyl)-indolyl]-phenyl or [1N-(1-4C-alkyl)-pyrazolyl]-phenyl. A further embodiment of the compounds according to the present invention relates to those compounds of formula I, in which R6 is [1N-(1-2C-alkyl)-indol-5-yl]-phenyl or [1N-(1-2C-alkyl)-pyrazol-4-yl]-phenyl. A further embodiment of the compounds according to the present invention relates to those compounds of formula I, in which R6 is 3-(1N-methyl-pyrazol-4-yl)-phenyl or 4-(1N-methyl-pyrazol-4-yl)-phenyl. A further embodiment of the compounds according to the present invention relates to those compounds of formula I, in which R6 is [1N-(1-2C-alkyl)-pyrazol-4-yl]-pyridinyl, such as e.g. 2-(1N-methyl-pyrazol-4-yl)-pyridin-4-yl or 6-(1N-methyl-pyrazol-4-yl)-pyridin-3-yl. A further embodiment of the compounds according to the present invention relates to those compounds of formula I, in which R6 is triazolyl-phenyl, which is substituted by R61 on the triazolyl moiety. A further embodiment of the compounds according to the present invention relates to those compounds of formula I, in which R6 is {1N-(R61)-[1,2,3]triazol-4-yl}-phenyl, such as e.g. 3-{1N-(R61)-[1,2,3]triazol-4-yl}-phenyl or 4-{1N-(R61)-[1,2,3]triazol-4-yl}-phenyl. A further embodiment of the compounds according to the present invention relates to those compounds of formula I, in which R61 is -T2-N(R611)R612.

A further embodiment of the compounds according to the present invention relates to those compounds of formula I, in which T2 is a bond. A further embodiment of the compounds according to the present invention relates to those compounds of formula I, in which T2 is 1-4C-alkylene, such as e.g. 1-2C-alkylene. A further embodiment of the compounds according to the present invention relates to those compounds of formula I, in which T2 is methylene. A further embodiment of the compounds according to the present invention relates to those compounds of formula I, in which T2 is dimethylene.

A further embodiment of the compounds according to the present invention relates to those compounds of formula I, in which T2 is trimethylene. A further embodiment of the compounds according to the present invention relates to those compounds of formula I, in which R611 and R612 are both hydrogen.

A further embodiment of the compounds according to the present invention relates to those compounds of formula I, in which R611 and R612 are both methyl. A further embodiment of the compounds according to the present invention relates to those compounds of formula I, in which R611 and R612 together and with inclusion of the nitrogen atom, to which they are attached, form a morpholino ring. A further embodiment of the compounds according to the present invention relates to those compounds of formula I, in which R611 and R612 together and with inclusion of the nitrogen atom, to which they are attached, form a 4N-methyl-piperazino ring. A further embodiment of the compounds according to the present invention relates to those compounds of formula I, in which R611 and R612 together and with inclusion of the nitrogen atom, to which they are attached, form a pyrrolidino ring. A further embodiment of the compounds according to the present invention relates to those compounds of formula I, in which R611 and R612 together and with inclusion of the nitrogen atom, to which they are attached, form a piperidino ring. A further embodiment of the compounds according to the present invention relates to those compounds of formula I, in which R61 is -O-T3-N(R613)R614.

A further embodiment of the compounds according to the present invention relates to those compounds of formula I, in which T3 is dimethylene. A further embodiment of the compounds according to the present invention relates to those compounds of formula I, in which T3 is trimethylene.

A further embodiment of the compounds according to the present invention relates to those compounds of formula I, in which R613 and R614 are both methyl. A further embodiment of the compounds according to the present invention relates to those compounds of formula I, in which R613 and R614 together and with inclusion of the nitrogen atom, to which they are attached, form a morpholino ring. A further embodiment of the compounds according to the present invention relates to those compounds of formula I, in which R613 and R614 together and with inclusion of the nitrogen atom, to which they are attached, form a 4N-methyl-piperazino ring. A further embodiment of the compounds according to the present invention relates to those compounds of formula I, in which R613 and R614 together and with inclusion of the nitrogen atom, to which they are attached, form a pyrrolidino ring. A further embodiment of the compounds according to the present invention relates to those compounds of formula I, in which R613 and R614 together and with inclusion of the nitrogen atom, to which they are attached, form a piperidino ring.

A further embodiment of the compounds according to the present invention relates to those compounds of formula I, in which R61 is -T4-Het3, in which
T4 is a bond, methylene, dimethylene or trimethylene, and
Het3 is 1 N-methyl-piperidin-4yl.

A further embodiment of the compounds according to the present invention relates to those compounds of formula I, in which R61 is -O-T5-Het4, in which
T5 is a bond, methylene, dimethylene or trimethylene, and
Het4 is 1 N-methyl-piperidin-4yl.

A further embodiment of the compounds according to the present invention relates to those compounds of formula I, in which R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl; and R7 is hydroxyl.

A further embodiment of the compounds according to the present invention relates to those compounds of formula I, in which R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl; and R7 is 2-aminophenyl.

A further embodiment of the compounds according to the present invention relates to those compounds of formula I, in which R6 is 4-(6-amino-pyridin-3-yl)-phenyl. A further embodiment of the compounds according to the present invention relates to those compounds of formula I, in which R6 is 4-[2-(4-methyl-piperazin-1-yl)-pyridin-4-yl)]-phenyl. A further embodiment of the compounds according to the present invention relates to those compounds of formula I, in which R6 is 3-[2-(4-methyl-piperazin-1-yl)-pyridin-4-yl)]-phenyl. A further embodiment of the compounds according to the present invention relates to those compounds of formula I, in which R6 is 4′-(2-morpholin-4-yl-ethyl)-biphenyl-3-yl. A further embodiment of the compounds according to the present invention relates to those compounds of formula I, in which R6 is 4′-dimethylaminomethyl-biphenyl-4-yl. A further embodiment of the compounds according to the present invention relates to those compounds of formula I, in which R6 is 5-[2-(4-methyl-piperazin-1-yl)-pyridin-4-yl)]-thiophen-2-yl. A further embodiment of the compounds according to the present invention relates to those compounds of formula I, in which R6 is 5-(4-dimethylaminomethyl-phenyl)-thiophen-2-yi.

A special embodiment of the compounds according to the present invention relates to those compounds of formula I, in which R1, R2, R3, R4 and R5 are all hydrogen, and R7 is hydroxyl. Another special embodiment of the compounds according to the present invention relates to those compounds of formula I, in which R1, R2, R3, R4 and R5 are all hydrogen, and R7 is 2-aminophenyl.

It is to be understood, that the present invention also includes any or all possible combinations and subsets of the embodiments defined herein afore.

Exemplary compounds according to this invention may include any one selected from
1. (E)-N-Hydroxy-3-{1-[4-(1-methyl-1H-indol-5-yl)-benzenesulfonyl]-1H-pyrrol-3-yl}-acrylamide,
2. (E)-N-Hydroxy-3-{1-[4-(1-methyl-1H-pyrazol-4-yl)-benzenesulfonyl]-1H-pyrrol-3-yl}-acrylamide,
3. (E)-N-Hydroxy-3-{1-[4-(6-methoxy-pyridin-3-yl)-benzenesulfonyl]-1H-pyrrol-3-yl}-acrylamide,
4. (E)-3-{1-[4-(6-Amino-pyridin-3-yl)-benzenesulfonyl]-1H-pyrrol-3-yl}-N-hydroxy-acrylamide,
5. (E)-N-(2-Amino-phenyl)-3-{1-[4-(6-methoxy-pyridin-3-yl)-benzenesulfonyl]-1H-pyrrol-3-yl}-acrylamide,
6. (E)-N-(2-Amino-phenyl)-3-{1-[4-(6-amino-pyridin-3-yl)-benzenesulfonyl]-1H-pyrrol-3-yl}-acrylamide,
7. (E)-N-(2-Amino-phenyl)-3-{1-[4-(1-methyl-1H-pyrazol-4-yl)-benzenesulfonyl]-1H-pyrrol-3-yl}-acrylamide,
8. (E)-N-Hydroxy-3-{1-[4′-(2-morpholin-4-yl-ethyl)-biphenyl-4-sulfonyl]-1H-pyrrol-3-yl}-acrylamide,
9. (E)-N-Hydroxy-3-{1-[3′-(2-morpholin-4-yl-ethyl)-biphenyl-4-sulfonyl]-1H-pyrrol-3-yl}-acrylamide,
10. (E)-3-{1-[3-(6-Amino-pyridin-3-yl)-benzenesulfonyl]-1H-pyrrol-3-yl}-N-hydroxy-acrylamide,
11. (E)-N-Hydroxy-3-{1-[3-(6-methoxy-pyridin-3-yl)-benzenesulfonyl]-1H-pyrrol-3-yl}-acrylamide,
12. (E)-N-Hydroxy-3-{1-[3-(1-methyl-1H-pyrazol-4-yl)-benzenesulfonyl]-1H-pyrrol-3-yl}-acrylamide,
13. (E)-N-Hydroxy-3-{1-[3-(1-methyl-1H-indol-5-yl)-benzenesulfonyl]-1H-pyrrol-3-yl}-acrylamide,
14. (E)-N-(2-Amino-phenyl)-3-{1-[3-(6-methoxy-pyridin-3-yl)-benzenesulfonyl]-1H-pyrrol-3-yl}-acrylamide,
15. (E)-N-(2-Amino-phenyl)-3-{1-[3-(1-methyl-1H-pyrazol-4-yl)-benzenesulfonyl]-1H-pyrrol-3-yl}-acrylamide,
16. (E)-N-Hydroxy-3-{1-[4′-(2-morpholin-4-yl-ethyl)-biphenyl-3-sulfonyl]-1H-pyrrol-3-yl}-acrylamide,
17. (E)-N-(2-Amino-phenyl)-3-{1-[3-(6-amino-pyridin-3-yl)-benzenesulfonyl]-1H-pyrrol-3-yl}-acrylamide,
18. (E)-N-(2-Amino-phenyl)-3-{1-[3′-(2-morpholin-4-yl-ethyl)-biphenyl-3-sulfonyl]-1H-pyrrol-3-yl}-acrylamide,
19. (E)-N-(2-Amino-phenyl)-3-{1-[4′-(2-morpholin-4-yl-ethyl)-biphenyl-3-sulfonyl]-1H-pyrrol-3-yl}-acrylamide,
20. (E)-N-Hydroxy-3-{1-[3′-(2-morpholin-4-yl-ethyl)-biphenyl-3-sulfonyl]-1H-pyrrol-3-yl}-acrylamide,
21. (E)-N-Hydroxy-3-[1-(2′-methanesulfonylamino-biphenyl-4-sulfonyl)-1H-pyrrol-3-yl]-acrylamide
22. (E)-N-hydroxy-3-[1-(3′-methanesulfonylamino-biphenyl-4-sulfonyl)-1H-pyrrol-3-yl]-acrylamide
23. (E)-N-Hydroxy-3-[1-(4′-methanesulfonylamino-biphenyl-4-sulfonyl)-1H-pyrrol-3-yl]-acrylamide
24. 4′-[3-((E)-2-Hydroxycarbamoyl-vinyl)-pyrrole-1-sulfonyl]-biphenyl-4-carboxylic acid (2-dimethylamino-ethyl)-amide,
25. 4′-[3-((E)-2-Hydroxycarbamoyl-vinyl)-pyrrole-1-sulfonyl]-biphenyl-3-carboxylic acid (2-dimethylamino-ethyl)-amide,
26. (E)-3-[1-(4′-Dimethylaminomethyl-biphenyl-4-sulfonyl)-1H-pyrrol-3-yl]-N-hydroxy-acrylamide,
27. (E)-3-[1-(2′-Dimethylaminomethyl-biphenyl-4-sulfonyl)-1H-pyrrol-3-yl]-N-hydroxy-acrylamide,
28. (E)-N-Hydroxy-3-(1-{4-[2-(4-methyl-piperazin-1-yl)-pyridin-4-yl]-benzenesulfonyl}-1H-pyrrol-3-yl)-acrylamide,
29. (E)-N-Hydroxy-3-{1-[4′-(toluene-4-sulfonylamino)-biphenyl-4-sulfonyl]-1H-pyrrol-3-yl}-acrylamide,
30. 3′-[3-((E)-2-Hydroxycarbamoyl-vinyl)-pyrrole-1-sulfonyl]-biphenyl-4-carboxylic acid (2-dimethylamino-ethyl)-amide,
31. (E)-N-Hydroxy-3-[1-(3′-morpholin-4-ylmethyl-biphenyl-3-sulfonyl)-1H-pyrrol-3-yl]-acrylamide,
32. (E)-N-Hydroxy-3-(1-{4′-[2-(4-methyl-piperazin-1-yl)-ethoxy]-biphenyl-3-sulfonyl}-1H-pyrrol-3-yl)-acrylamide,
33. (E)-N-Hydroxy-3-(1-{3-[2-(4-methyl-piperazin-1-yl)-pyridin-4-yl]-benzenesulfonyl}-1H-pyrrol-3-yl)-acrylamide,
34. (E)-N-Hydroxy-3-{1-[4′-(2-morpholin-4-yl-ethoxy)-biphenyl-3-sulfonyl]-1H-pyrrol-3-yl}-acrylamide,
35. (E)-N-(2-Amino-phenyl)-3-{1-[4-(1-benzyl-1H-pyrazol-4-yl)-benzenesulfonyl]-1H-pyrrol-3-yl}-acrylamide,
36. (E)-N-Hydroxy-3-[1-(4′-morpholin-4-ylmethyl-biphenyl-3-sulfonyl)-1H-pyrrol-3-yl]-acrylamide,
37. (E)-3-[1-(4′-Dimethylaminomethyl-biphenyl-3-sulfonyl)-1H-pyrrol-3-yl]-N-hydroxy-acrylamide,
38. (E)-N-Hydroxy-3-{1-[4′-(3-morpholin-4-yl-propoxy)-biphenyl-3-sulfonyl]-1H-pyrrol-3-yl}-acrylamide,
39. (E)-N-(2-Amino-phenyl)-3-[1-(4′-dimethylsulfamoyl-biphenyl-4-sulfonyl)-1H-pyrrol-3-yl]-acrylamide,
40. (E)-3-[1-(3′-Acetylamino-biphenyl-4-sulfonyl)-1H-pyrrol-3-yl]-N-(2-amino-phenyl)-acrylamide,
41. (E)-3-[1-(2′-Dimethylaminomethyl-biphenyl-3-sulfonyl)-1H-pyrrol-3-yl]-N-hydroxy-acrylamide,
42. (E)-N-Hydroxy-3-(1-{5-[2-(4-methyl-piperazin-1-yl)-pyridin-4-yl]-thiophene-2-sulfonyl}-1H-pyrrol-3-yl)-acrylamide,
43. (E)-N-Hydroxy-3-{1-[4′-(2-pyrrolidin-1-yl-ethoxy)-biphenyl-3-sulfonyl]-1H-pyrrol-3-yl}-acrylamide,
44. 4′-{3-[(E)-2-(2-Amino-phenylcarbamoyl)-vinyl]-pyrrole-1-sulfonyl}-biphenyl-3-carboxylic acid (2-dimethylamino-ethyl)-amide,
45. (E)-N-Hydroxy-3-{1-[4′-(3-morpholin-4-yl-propyl)-biphenyl-3-sulfonyl]-1H-pyrrol-3-yl}-acrylamide,
46. (E)-3-{1-[5-(4-Dimethylaminomethyl-phenyl)-thiophene-2-sulfonyl]-1H-pyrrol-3-yl}-N-hydroxy-acrylamide,
47. (E)-N-(2-Amino-phenyl)-3-[1-(4′-dimethylaminomethyl-biphenyl-4-sulfonyl)-1H-pyrrol-3-yl]-acrylamide,
48. (E)-N-(2-Amino-phenyl)-3-(1-{4-[2-(4-methyl-piperazin-1-yl)-pyridin-4-yl]-benzenesulfonyl}-1H-pyrrol-3-yl)-acrylamide,
49. (E)-3-[1-(4′-Acetylamino-biphenyl-4-sulfonyl)-1H-pyrrol-3-yl]-N-(2-amino-phenyl)-acrylamide,
50. (E)-N-Hydroxy-3-{1-[5-(3-morpholin-4-ylmethyl-phenyl)-thiophene-2-sulfonyl]-1H-pyrrol-3-yl}-acrylamide,
51. (E)-N-(2-Amino-phenyl)-3-[1-(3′-hydroxymethyl-biphenyl-4-sulfonyl)-1H-pyrrol-3-yl]-acrylamide,
52. (E)-N-(2-Amino-phenyl)-3-{1-[4-(3,5-dimethyl-isoxazol-4-yl)-benzenesulfonyl]-1H-pyrrol-3-yl}-acrylamide,
53. (E)-N-(2-Amino-phenyl)-3-[1-(4′-methanesulfonylamino-biphenyl-4-sulfonyl)-1H-pyrrol-3-yl]-acrylamide,
54. (E)-N-Hydroxy-3-{1-[5-(4-morpholin-4-ylmethyl-phenyl)-thiophene-2-sulfonyl]-1H-pyrrol-3-yl}-acrylamide,
55. (E)-N-Hydroxy-3-[1-(5-{4-[2-(4-methyl-piperazin-1-yl)-ethoxy]-phenyl}-thiophene-2-sulfonyl)-1H-pyrrol-3-yl]-acrylamide,
56. (E)-N-Hydroxy-3-(1-{5-[4-(2-morpholin-4-yl-ethoxy)-phenyl]-thiophene-2-sulfonyl}-1H-pyrrol-3-yl)-acrylamide,
57. (E)-N-Hydroxy-3-(1-{5-[4-(3-morpholin-4-yl-propoxy)-phenyl]-thiophene-2-sulfonyl}-1H-pyrrol-3-yl)-acrylamide,
58. (E)-N-Hydroxy-3-(1-{4′-[(2-methoxy-ethylamino)-methyl]-biphenyl-3-sulfonyl}-1H-pyrrol-3-yl)-acrylamide,
59. (E)-N-(2-Amino-phenyl)-3-[1-(3′-methanesulfonylamino-biphenyl-4-sulfonyl)-1H-pyrrol-3-yl]-acrylamide,
60. (E)-Hydroxy-3-{1-[5-(1-methyl-1H-pyrazol-4-yl)-thiophene-2-sulfonyl]-1H-pyrrol-3-yl}-acrylamide,
61. (E)-N-Hydroxy-3-(1-{5-[4-(2-morpholin-4-yl-ethyl)-phenyl]-thiophene-2-sulfonyl}-1H-pyrrol-3-yl)-acrylamide,
62. (E)-N-Hydroxy-3-{1-[4′-(4-methyl-piperazin-1-ylmethyl)-biphenyl-3-sulfonyl]-1H-pyrrol-3-yl}-acrylamide, and
63. (E)-3-[1-(4′-Cyclopropylaminomethyl-biphenyl-3-sulfonyl)-1H-pyrrol-3-yl]-N-hydroxy-acrylamide,
and the salts thereof.

Further on, exemplary compounds according to this invention may also include any one selected from
64. (E)-N-Hydroxy-3-[1-(3′-morpholin-4-ylmethyl-biphenyl-4-sulfonyl)-1H-pyrrol-3-yl]-acrylamide,
65. (E)-3-[1-(4-Benzo[1,3]dioxol-5-yl-benzenesulfonyl)-1H-pyrrol-3-yl]-N-hydroxy-acrylamide,
66. (E)-3-[1-(3′-Amino-biphenyl-4-sulfonyl)-1H-pyrrol-3-yl]-N-hydroxy-acrylamide,
67. (E)-N-Hydroxy-3-[1-(4′-hydroxy-biphenyl-4-sulfonyl)-1H-pyrrol-3-yl]-acrylamide,
68. (E)-N-Hydroxy-3-(1-{4′-[2-(1-methyl-piperidin-4-yl)-ethoxy]-biphenyl-4-sulfonyl}-1H-pyrrol-3-yl)-acrylamide,
69. (E)-3-[1-(3′-Dimethylamino-biphenyl-4-sulfonyl)-1H-pyrrol-3-yl]-N-hydroxy-acrylamide,
70. (E)-3-{1-[4-(2,3-Dihydro-benzofuran-5-yl)-benzenesulfonyl]-1H-pyrrol-3-yl}-N-hydroxy-acrylamide,
71. (E)-N-Hydroxy-3-[1-(4′-morpholin-4-yl-biphenyl-4-sulfonyl)-1H-pyrrol-3-yl]-acrylamide,
72. (E)-N-Hydroxy-3-{1-[3′-(3-pyrrolidin-1-yl-propoxy)-biphenyl-4-sulfonyl]-1H-pyrrol-3-yl}-acrylamide,
73. (E)-N-Hydroxy-3-(1-{3′-[3-(4-methyl-piperazin-1-yl)-propoxy]-biphenyl-4-sulfonyl}-1H-pyrrol-3-yl)-acrylamide,
74. (E)-N-Hydroxy-3-{1-[3′-(3-morpholin-4-yl-propoxy)-biphenyl-4-sulfonyl]-1H-pyrrol-3-yl}-acrylamide,
75. (E)-N-Hydroxy-3-[1-(3′-morpholin-4-ylmethyl-biphenyl-4-sulfonyl)-1H-pyrrol-3-yl]-acrylamide,
76. (E)-N-Hydroxy-3-(1-{4′-[2-(4-methyl-piperazin-1-yl)-ethoxy]-biphenyl-4-sulfonyl}-1H-pyrrol-3-yl)-acrylamide,
77. (E)-N-Hydroxy-3-{1-[4′-(2-morpholin-4-yl-ethoxy)-biphenyl-4-sulfonyl]-1H-pyrrol-3-yl}-acrylamide,
78. (E)-N-Hydroxy-3-{1-[4′-(3-morpholin-4-yl-propoxy)-biphenyl-4-sulfonyl]-1H-pyrrol-3-yl}-acrylamide,
79. (E)-N-Hydroxy-3-(1-{4′-[3-(4-methyl-piperazin-1-yl)-propoxy]-biphenyl-4-sulfonyl}-1H-pyrrol-3-yl)-acrylamide,
80. (E)-N-Hydroxy-3-{1-[3′-(2-pyrrolidin-1-yl-ethoxy)-biphenyl-4-sulfonyl]-1H-pyrrol-3-yl}-acrylamide,
81. (E)-N-Hydroxy-3-{1-[4′-(3-pyrrolidin-1-yl-propoxy)-biphenyl-4-sulfonyl]-1H-pyrrol-3-yl}-acrylamide,
82. (E)-N-Hydroxy-3-[1-(4′-methoxy-biphenyl-4-sulfonyl)-1H-pyrrol-3-yl]-acrylamide,
83. (E)-N-Hydroxy-3-(1-{4-[1-(2-morpholin-4-yl-ethyl)-1H-[1,2,3]triazol-4-yl]-benzenesulfonyl}-1H-pyrrol-3-yl)-acrylamide,
84. (E)-3-[1-(4′-Cyclopentylaminomethyl-biphenyl-4-sulfonyl)-1H-pyrrol-3-yl]-N-hydroxy-acrylamide,
85. (E)-N-Hxydroxy-3-[1-(3′-trifluoromethyl-biphenyl-4-sulfonyl)-1H-pyrrol-3-yl]-acrylamide,
86. (E-3-{1-[5-(3-Dimethylaminomethyl-phenyl)-thiophene-2-sulfonyl]-1H-pyrrol-3-yl}-N-hydroxy-acrylamide,
87. (E)-3-[l-(3′-Dimethylaminomethyl-biphenyl-3-sulfonyl)-lH-pyrrol-3-yl]-N-hydroxy-acrylamide,
88. (E)-N-Hydroxy-3-{1-[4′-(2-morpholin-4-yl-ethyl)-biphenyl-3-sulfonyl]-1H-pyrrol-3-yl}-acrylamide,
89. (E)-N-(2-Amino-phenyl)-3-{1-[6-(4-dimethylaminomethyl-phenyl)-pyridine-3-sulfonyl]-1H-pyrrol-3-yl}-acrylamide,
90. (E)-N-Hydroxy-3-{1-[5-(2-methyl-thiazol-4-yl)-thiophene-2-sulfonyl]-1H-pyrrol-3-yl}-acrylamide,
91. (E)-3-[1-(4′-Aminomethyl-biphenyl-3-sulfonyl)-1H-pyrrol-3-yl]-N-hydroxy-acrylamide,
92. (E)-N-Hydroxy-3-(1-{6-[4-(2-pyrrolidin-1-yl-ethoxy)-phenyl]-pyridine-3-sulfonyl}-1H-pyrrol-3-yl)-acrylamide,
93. (E)-3-[1-(4′-Aminomethyl-biphenyl-4-sulfonyl)-1H-pyrrol-3-yl]-N-(2-amino-phenyl)-acrylamide,
94. (E)-3-{1-[5-(3-Aminomethyl-phenyl)-thiophene-2-sulfonyl]-1H-pyrrol-3-yl}-N-hydroxy-acrylamide,
95. (E)-N-(2-Amino-phenyl)-3-{1-[5-(4-dimethylaminomethyl-phenyl)-thiophene-2-sulfonyl]-1H-pyrrol-3-yl}-acrylamide,
96. (E)-N-(2-Amino-phenyl)-3-[1-(3′-dimethylaminomethyl-biphenyl-4-sulfonyl)-1H-pyrrol-3-yl]-acrylamide,
97. (E)-3-{1-[4′-(Acetylamino-methyl)-biphenyl-4-sulfonyl]-1H-pyrrol-3-yl}-N-(2-aminophenyl)-acrylamide,
98. (E)-N-(2-Amino-phenyl)-3-{1-[4′-(methanesulfonylamino-methyl)-biphenyl-4-sulfonyl]-1H-pyrrol-3-yl}-acrylamide,
99. (E)-N-Hydroxy-3-(1-{5-[4-(2-pyrrolidin-1-yl-ethoxy)-phenyl]-thiophene-2-sulfonyl}-1H-pyrrol-3-yl)-acrylamide,
100. (E)-3-{1-[5-(4-Dimethylsulfamoyl-phenyl)-thiophene-2-sulfonyl]-1H-pyrrol-3-yl}-N-hydroxy-acrylamide,
101. (E)-N-(2-Amino-phenyl)-3-[1-(4′-methanesulfonylamino-biphenyl-3-sulfonyl)-1H-pyrrol-3-yl]-acrylamide,
102. (E)-N-(2-Amino-phenyl)-3-[1-(4′-dimethylaminomethyl-biphenyl-3-sulfonyl)-1H-pyrrol-3-yl]-acrylamide,
103. (E)-N-Hydroxy-3-{1-[2′-(4-methyl-piperazin-1-yl)-[2,4′]bipyridinyl-5-sulfonyl]-1H-pyrrol-3-yl}-acrylamide,
104. (E)-N-(2-Amino-phenyl)-3-{1-[5-(1-methyl-1H-pyrazol-4-yl)-thiophene-2-sulfonyl]-1H-pyrrol-3-yl}-acrylamide,
105. (E)-3-{1-[6-(4-Dimethylaminomethyl-phenyl)-pyridine-3-sulfonyl]-1H-pyrrol-3-yl}-N-hydroxy-acrylamide,
106. (E)-N-(2-Amino-phenyl)-3-(1-{5-[2-(4-methyl-piperazin-1-yl)-pyridin-4-yl]-thiophene-2-sulfonyl}-1H-pyrrol-3-yl)-acrylamide,
107. (E)-N-(2-Amino-phenyl)-3-[1-(4′-morpholin-4-ylmethyl-biphenyl-4-sulfonyl)-1H-pyrrol-3-yl]-acrylamide,
108. (E)-N-(2-Amino-phenyl)-3-{1-[4′-(2-pyrrolidin-1-yl-ethoxy)-biphenyl-4-sulfonyl]-1H-pyrrol-3-yl}-acrylamide,
109. (E)-N-Hydroxy-3-(1-{4-[1-(2-piperidin-1-yl-ethyl)-1H-[1,2,3]triazol-4-yl]-benzenesulfonyl}-1H-pyrrol-3-yl)-acrylamide,
110. (E)-3-[1-(3′-Dimethylaminomethyl-biphenyl-3-sulfonyl)-1H-pyrrol-3-yl]-N-hydroxy-acrylamide,
111. (E)-N-(2-Amino-phenyl)-3-(1-{5-[4-(methynesulfonylamino-methyl)-phenyl]-thiophene-2-sulfonyl}-1H-pyrrol-3-yl)-acrylamide,
112. (E)-N-(2-Amino-phenyl)-3-{1-[3′-(methanesulfonylamino-methyl)-biphenyl-3-sulfonyl]-1H-pyrrol-3-yl}-acrylamide,
113. (E)-3-(1-{5-[4-(Acetylamino-methyl)-phenyl]-thiophene-2-sulfonyl}-1H-pyrrol-3-yl)-N-(2-amino-phenyl)-acrylamide,
114. (E)-N-(2-Amino-phenyl)-3-{1-[5-(3-dimethylaminomethyl-phenyl)-thiophene-2-sulfonyl]-1H-pyrrol-3-yl}-acrylamide,
115. (E)-N-(2-Amino-phenyl)-3-[1-(3′-dimethylaminomethyl-biphenyl-3-sulfonyl)-1H-pyrrol-3-yl]-acrylamide,
116. (E)-3-[1-(3′-Dimethylaminomethyl-biphenyl-4-sulfonyl)-1H-pyrrol-3-yl]-N-hydroxy-acrylamide,
117. (E)-3-{1-[5-(3-Dimethylaminomethyl-phenyl)-thiophene-2-sulfonyl]-1H-pyrrol-3-yl}-N-hydroxy-acrylamide,
118. (E)-3-{1-[3′-(Acetylamino-methyl)-biphenyl-3-sulfonyl]-1H-pyrrol-3-yl}-N-(2-aminophenyl)-acrylamide,
119. (E)-N-(2-Amino-phenyl)-3-{1-[6-(1-methyl-1H-pyrazol-4-yl)-pyridine-3-sulfonyl]-1H-pyrrol-3-yl}-acrylamide,
120. (E)-N-Hydroxy-3-{1-[6-(1-methyl-1H-pyrazol-4-yl)-pyridine-3-sulfonyl]-1H-pyrrol-3-yl}-acrylamide, and
121. (E)-3-{1-[6-(3-Dimethylaminomethyl-phenyl)-pyridine-3-sulfonyl]-1H-pyrrol-3-yl}-N-hydroxy-acrylamide,
and the salts thereof.

In an embodiment of the foregoing, exemplary compounds according to this invention may especially include any one selected from the group consisting of the compounds 2, 4, 7, 16, 26, 28, 32, 33, 38, 42 and 46 as mentioned afore, and the salts thereof.

As used herein, 4SC-202 and (E)-N-(2-aminophenyl)-3-(1-((4-(1-methyl-1H-pyrazol-4-yl)phenyl)sulfonyl)-1H-pyrrol-3-yl)acrylamide (its chemical name) are used interchangeably and both refer to a compound of the following formula:

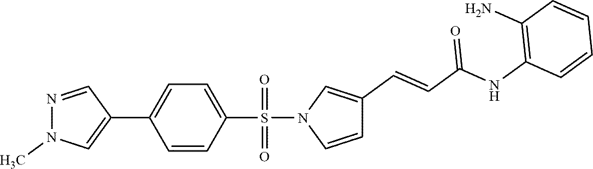 [SMILES: Cn1cc(-c2ccc(S(=O)(=O)n3ccc(/C=C/C(=O)Nc4ccccc4N)c3)cc2)cn1]

### Hydroxamate Type HDAC Inhibitors

In some embodiments, the HDAC inhibitor is a hydroxamic acid derivative. In some embodiments, the HDAC inhibitor is a hydroxamic acid derivative of formula (II):

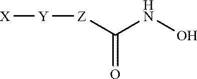

wherein:
Y is —NHC(O)—, —C(O)—, —NH—, —C(O)NH—, —NR¹, —OC(O)NH—, or —NHSO₂—;
R¹ is C₁-C₆ alkyl optionally substituted by 1-4 hydroxyl groups;
X is C₁-C₆ alkyl, phenyl optionally substituted by 1-4 —NR²R³ groups,
-(C₁-C₆ alkylene)-heteroaryl optionally substituted by 1-4 C₁-C₆ alkyl or halogen groups,
-(C₁-C₆ alkylene)-arylene optionally substituted by 1-4 —CH₂NR⁴R⁵ groups, or heteroaryl optionally substituted by 1-4 —NR⁶R⁷ or —CH₂NR⁴R⁵ groups;
R², R³, R⁴, and R⁵ are independently C₁-C₆ alkyl;
R^{s} and R⁷ are independently aryl or heteroaryl;
Z is C₁-C₆ alkylene, C₂-C₆ alkenylene optionally substituted by 1-4 C₁-C₆ alkyl groups, or

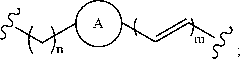

n is 0, 1, or 2;
m is 0 or 1; and
A is arylene, -O-arylene, heteroarylene optionally substituted by 1-4 C₁-C₆ alkyl groups, -heterocyclylene-heteroarylene-, or -cycloalkylene-heteroarylene,
or a salt thereof.

In some embodiments, Y is —NHC(O)— or —C(O)NH—. In other embodiments, Y is —C(O)—, —NH—, —NR¹, or —OC(O)NH—. In further embodiments, Y is —NHSO₂—.

In some embodiments, R¹ is methyl, ethyl or propyl. In some embodiments, R¹ is ethyl. In some embodiments, R¹ is ethyl substituted with one hydroxyl group (e.g., -CH₂CH₂OH).

In some embodiments, X is C₁-C₆ alkyl. In some embodiments, X is methyl, ethyl, propyl, or butyl. In some embodiments, X is ethyl.

In some embodiments, X is phenyl optionally substituted by 1-4 -NR²R³ groups. In some embodiments, X is unsubstituted phenyl. In some embodiments, X is phenyl substituted by one -NR²R³ group. In some embodiments, X is phenyl substituted by one -NR²R³ group, wherein R² and R³ are independently methyl, ethyl, or propyl. In some embodiments, X is phenyl substituted by one -NR²R³ group, wherein R² and R³ are each methyl.

In some embodiments, X is -(C₁-C₆ alkylene)-heteroaryl optionally substituted by 1-4 C₁-C₆ alkyl. In some embodiments, X is -(C₁-C₃ alkylene)-heteroaryl optionally substituted by one C₁-C₆ group. In some embodiments, X is -(ethylene)-heteroaryl optionally substituted by one methyl, ethyl, or propyl group. In some embodiments, X is -(ethylene)-indolyl. In some embodiments, X is -(ethylene)-indolyl substituted by one methyl group. In some embodiments, X is -(methylene)-heteroaryl optionally substituted by one methyl, ethyl, or propyl group. In some embodiments, X is -(methylene)-indolyl substituted by one methyl group. In some embodiments, X is -(C₁-C₆ alkylene)-heteroaryl optionally substituted by 1-4 halogen groups. In some embodiments, X is -(C₁-C₂ alkylene)-heteroaryl optionally substituted by one halogen group. In some embodiments, X is -(methylene)-heteroaryl optionally substituted by one chloro, bromo, or fluoro group. In some embodiments, X is -(methylene)-quinolinyl substituted by one fluoro group.

In some embodiments, X is -(C₁-C₆ alkylene)-arylene optionally substituted by 1-4 —CH₂NR⁴R⁵ groups. In some embodiments, X is -(C₁-C₂ alkylene)-arylene optionally substituted by one -CH₂NR⁴R⁵ group. In some embodiments, X is -(methylene)-arylene substituted by one -CH₂NR⁴R⁵ group, wherein R⁴ and R⁵ are independently methyl, ethyl, or propyl. In some embodiments, X is -(methylene)-naphthyl substituted by one —CH₂NR⁴R⁵ group, wherein R⁴ and R⁵ are each ethyl.

In some embodiments, X is heteroaryl optionally substituted by 1-4 -NR⁶R⁷ or -CH₂NR⁴R⁵ groups. In some embodiments, X is heteroaryl optionally substituted by one -NR⁶R⁷ group, wherein R⁶ and R⁷ are both aryl. In some embodiments, X is pyrimidyl substituted by one NR⁶R⁷ group, wherein R⁶ and R⁷ are both phenyl. In some embodiments, X is heteroaryl optionally substituted by one -CH₂NR⁴R⁵ group, wherein R⁴ and R⁵ are independently methyl, ethyl, or propyl. In some embodiments, X is a fused bicyclic heteroaromatic ring system. In some embodiments, X is benzofuranyl substituted by one -CH₂NR⁴R⁵ group, wherein R⁴ and R⁵ are each methyl.

In some embodiments, Z is C₁-C₆ alkylene. In some embodiments, Z is C₄-C₆ alkylene. In some embodiments, Z is C₆ alkylene (e.g., hexylene, —(CH₂)₆—).

In some embodiments, Z is C₂-C₆ alkenylene optionally substituted by 1-4 C₁-C₆ alkyl groups.

In some embodiments, Z is C₄-C₆ alkenylene optionally substituted by 2-3 C₁-C₆ alkyl groups.

In some embodiments, Z is C₅ alkenylene substituted by two groups independently selected from the group consisting of methyl, ethyl, and propyl. In some embodiments, Z is C₅ alkenylene having two unsaturated bonds and substituted by two methyl groups.

In some embodiments, Z is

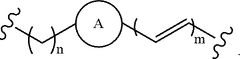

In some embodiments, A is arylene.

In some embodiments, A is phenylene, m is 1, and n is 0. In some embodiments, A is phenylene, m is 1, and n is 1. In some embodiments, A is phenylene, m is 0, and n is 0. In some embodiments, A is -O-arylene. In some embodiments, A is -O-phenylene, m is 0, and n is 2. In some embodiments, A is heteroarylene optionally substituted by 1-4 C₁-C₆ alkyl groups. In some embodiments, A is benzimidazolylene substituted by one butyl group, m is 1, and n is 2. In some embodiments, A is -heterocyclylene-heteroarylene-. In some embodiments, A is -piperidinylene-pyrimidinylene-, m is 0, and n is 1. In some embodiments, A is -cycloalkylene-heteroarylene. In some embodiments, A is -(bicyclo[3.1.0]hexanylene)-(pyrimidinylene), m is 0, and n is 0.

In some embodiments, the hydroxamic acid derivative of formula (II), or a salt thereof, is vorinostat, trichostatin A, belinostat, panobinostat, ricolinostat, pracinostat, givinostat, abexinostat, quisinostat, CHR-3996, or dacinostat, or a salt thereof (chemical structures shown in Table 1). In some embodiments, the hydroxamic acid derivative of formula (II), or a salt thereof, is vorinostat, belinostat, or panobinostat, or a salt thereof.

In some embodiments, the compound of formula (II) is formulated as a salt thereof. Suitable salts include all acid addition salts or all salts with bases. Exemplary salts include hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid such as (-)-L-malic acid or (+)-D-malic acid, fumaric acid, succinic acid, oxalic acid, tartaric acid such as (+)-L-tartaric acid or (-)-D-tartaric acid or meso-tartaric acid, embonic acid, stearic acid, toluenesulphonic acid, methanesulphonic acid or 3-hydroxy-2-naphthoic acid.

TABLE 1

| | |
|---|---|
| Nomenclature(s) | Chemical Structure |
| MK-0603; Vorinostat (ZOLINZA); Suberanilohydroxamic acid (SAHA) | 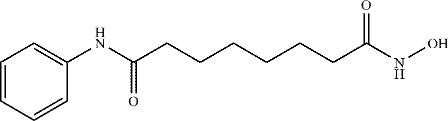 [SMILES: O=C(CCCCCCC(=O)Nc1ccccc1)NO] |
| TSA; Trichostatin A; 7-(4-(dimethylamino)phenyl)-N-hydroxy-4,6-dimethyl-7-oxo-2,4-heptadienamide | 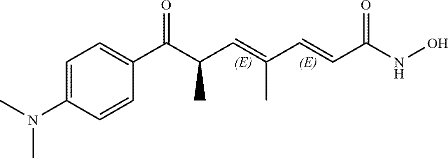 |
| PXD-101; Belinostat (BELEODAQ); N-hydroxy-3-(3-((phenylamino)sulfonyl)phenyl)-2-propenamide | 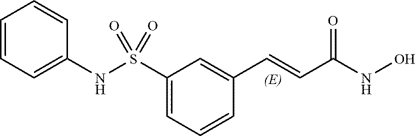 |
| LBH-589; Panobinostat (FARYDAK); (2E)-N-hydroxy-3-(4-(((2-(2-methyl-1H-indol-3-yl)ethyl)amino)methyl)phenyl)-2-propenamide | 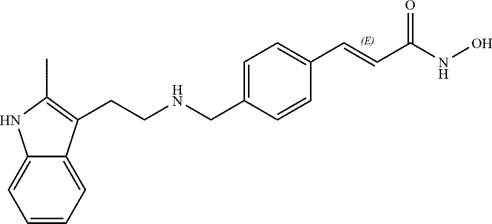 [SMILES: Cc1[nH]c2ccccc2c1CCNCc1ccc(/C=C/C(=O)NO)cc1] |
| ACY-1215; Ricolinostat; 2-(diphenylamino)-N-(7-(hydroxyamino)-7-oxoheptyl)-5-pyrimidinecarboxamide | |
| SB939; Pracinostat; (2E)-3-(2-butyl-1-(2-(diethylamino)ethyl)-1H-benzimidazol-5-yl)-N-hydroxy-2-propenamide | 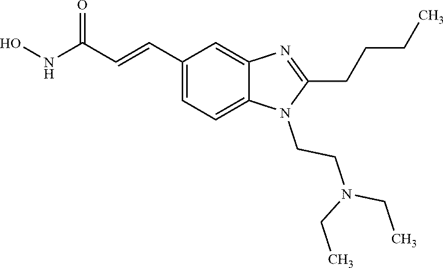 [SMILES: CCCCc1nc2cc(/C=C/C(=O)NO)ccc2n1CCN(CC)CC] |
| ITF-2357; Givinostat; (6-((Diethylamino)methyl)naphthalen-2-yl)methyl(4-(hydroxycarbamoyl)phenyl)carbamate | 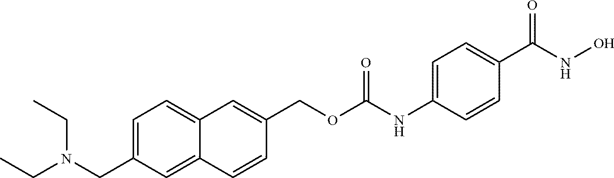 [SMILES: CCN(CC)Cc1ccc2cc(COC(=O)Nc3ccc(C(=O)NO)cc3)ccc2c1] |
| PCI-024781; Abexinostat; 3-((dimethylamino)methyl)-N-(2-(4-((hydroxyamino)carbonyl)phenoxy)ethyl)-2-benzofurancarboxamide | 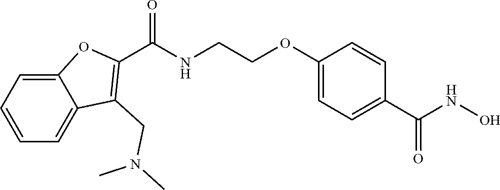 [SMILES: CN(C)Cc1c(C(=O)NCCOc2ccc(C(=O)NO)cc2)oc2ccccc12] |
| JNJ-26481585; Quisinostat; N-hydroxy-2-(4-((((1-methyl-1H-indol-3-yl)methyl)amino)methyl)-1-piperidinyl)-5-pyrimidinecarboxamide | 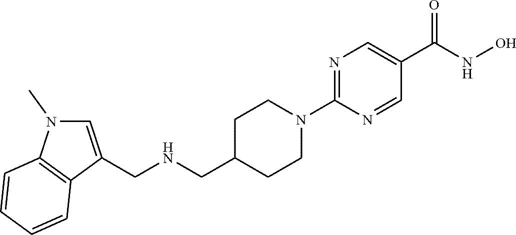 [SMILES: Cn1cc(CNCC2CCN(c3ncc(C(=O)NO)cn3)CC2)c2ccccc21] |
| CHR-3996 | 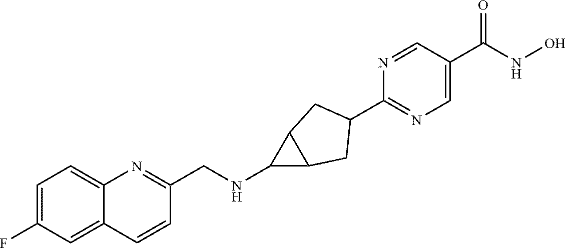 [SMILES: O=C(NO)c1cnc(C2CC3C(C2)C3NCc2ccc3cc(F)ccc3n2)nc1] |
| LAQ-824; Dacinostat; N-hydroxy-3-(4-(((2-hydroxyethyl)(2-(1H-indol-2-yl)ethyl)amino)methyl)phenyl)-2-propenamide | 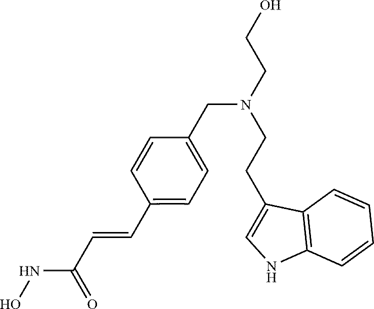 [SMILES: O=C(/C=C/c1ccc(CN(CCO)CCc2c[nH]c3ccccc23)cc1)NO] |

In some embodiments, the HDAC inhibitor is a benzamide derivative of formula (III):

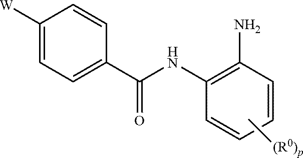

wherein:
R⁰ is halogen;
p is 0, 1, or 2;
W is —NHC(O)(C₁—C₆ alkyl) or —CH₂NH—V; and
V is —CO₂—CH₂—(heteroaryl), —C(O)—CH═CH—(heteroaryl), or heteroaryl optionally substituted by 1-3 heteroaryl groups,
or a salt thereof.

In some embodiments, R⁰ is chloro, bromo, or fluoro. In some embodiments, R⁰ is fluoro. In some embodiments, p is 0. In other embodiments, p is 1. In further embodiments, p is 2. In some embodiments, R⁰ is fluoro and p is 1. In some embodiments, R⁰ is fluoro attached at the para position and p is 1.

In some embodiments, W is -NHC(O)(C₁-C₆ alkyl). In some embodiments, W is —NHC(O)(C₁—C₃ alkyl). In some embodiments, W is —NHC(O)CH₃.

In some embodiments, W is —CH₂NH—V, wherein V is —CO₂—CH₂—(heteroaryl). In some embodiments, W is —CH₂NH—V, wherein V is —CO₂—CH₂—(pyridyl).

In some embodiments, W is —CH₂NH—V, wherein V is —C(O)—CH═CH—(heteroaryl). In some embodiments, W is —CH₂NH—V, wherein V is —C(O)—CH═CH—(pyridyl).

In some embodiments, W is —CH₂NH—V, wherein V is heteroaryl optionally substituted by 1-3 heteroaryl groups. In some embodiments, W is —CH₂NH—V, wherein V is heteroaryl optionally substituted by 1 heteroaryl group. In some embodiments, W is —CH₂NH—V, wherein V is pyrimidinyl substituted by 1 pyridyl group.

In some embodiments, the benzamide derivative of formula (III), or a salt thereof, is entinostat, mocetinostat, tacedinaline, or tucidinostat, or a salt thereof (chemical structures shown in Table 2). In some embodiments, the benzamide derivative of formula (III), or a salt thereof, is mocetinostat or entinostat, or a salt thereof.

In some embodiments, the compound of formula (III) is formulated as a salt thereof. Suitable salts include all acid addition salts or all salts with bases. Exemplary salts include hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid such as (-)-L-malic acid or (+)-D-malic acid, fumaric acid, succinic acid, oxalic acid, tartaric acid such as (+)-L-tartaric acid or (-)-D-tartaric acid or meso-tartaric acid, embonic acid, stearic acid, toluenesulphonic acid, methanesulphonic acid or 3-hydroxy-2-naphthoic acid.

TABLE 2

| | |
|---|---|
| Nomenclature(s) | Chemical Structure |
| MS-275 (SNDX-275); Entinostat; N-(2-Aminophenyl)-4-(N-(pyridin-3-ylmethoxycarbonyl)aminomethyl)benzamide | 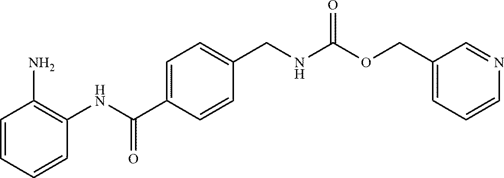 [SMILES: Nc1ccccc1NC(=O)c1ccc(CNC(=O)OCc2cccnc2)cc1] |
| MGCD-0103; Mocetinostat; N-(2-Aminophenyl)-4-((4-pyridin-3-ylpyrimidin-2-ylamino)methyl)benzamide | 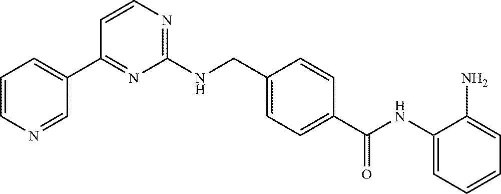 [SMILES: Nc1ccccc1NC(=O)c1ccc(CNc2nccc(-c3cccnc3)n2)cc1] |
| Cl994; Tacedinaline; 4-(Acetylamino)-N-(2-aminophenyl)benzamide | 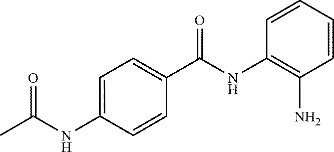 [SMILES: CC(=O)Nc1ccc(C(=O)Nc2ccccc2N)cc1] |
| Chidamide (CS-055); Tucidinostat; N-(2-amino-4-fluorophenyl)-4-((((2E)-1-oxo-3-(3-pyridinyl)-2-propen-1-yl)amino)methyl)-benzamide | 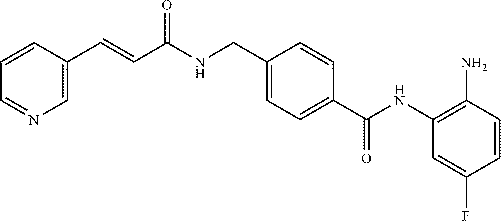 [SMILES: Nc1ccc(F)cc1NC(=O)c1ccc(CNC(=O)/C=C/c2cccnc2)cc1] |

Suitable salts for the HDAC inhibitor are acid addition salts or salts with bases. Particular mention may be made of the pharmacologically tolerable inorganic and organic acids and bases customarily used in pharmacy. Those suitable are, on the one hand, water-insoluble and, particularly, water-soluble acid addition salts, the acids being employed in salt preparation in an equimolar quantitative ratio or one differing therefrom, particularly in an equimolar quantitative ratio. On the other hand, salts with bases are - depending on substitution - also suitable, the bases being employed in salt preparation in an equimolar quantitative ratio or one differing therefrom. Pharmacologically intolerable salts, which can be obtained, for example, as process products during the preparation of the HDAC inhibitor on an industrial scale, are converted into pharmacologically tolerable salts by processes known to the person skilled in the art. According to the invention, the HDAC inhibitor as well as its salts may contain, e.g. when isolated in crystalline form, varying amounts of solvents. Included within the scope of the present invention are therefore all solvates and in particular all hydrates of the HDAC inhibitor as well as all solvates and in particular all hydrates of the HDAC inhibitor, in particular such solvates or hydrates comprising about 0.5, 1 or 2 solvate or water molecules per molecule of the HDAC inhibitor or salts thereof.

Particular salts in the context of the present invention are the salts of 4SC-202 with, HBr, methansulfonic acid, hemiethane-1,2-disulfonic acid, benzenesulfonic acid, toluenesulfonic acid and 2-naphthalenesulfonic acid, more particularly toluenesulfonic acid, in particular in a molar ratio of about 1:1.

The HDAC inhibitor, in particular 4SC-202 and salts thereof can be prepared, for example, as described in detail in WO 2006/097474 A1 and WO 2009/112522 A1, respectively.

The HDAC inhibitor, in particular 4SC-202 and salts thereof can be prepared, for example, as described in detail in WO 2006/097474 A1 and WO 2009/112522 A1, respectively.

The ability of the present immune checkpoint inhibitors which are biologicals, e.g. antibodies, to bind to the present immune checkpoints can be assessed by in vitro/ in vivo and/or cell-based assays either using purified domains of the target proteins or cells using ELISA or flow cytometry methods with a wide array of assays, e.g. the ELISA assay as described herein.

The biological and medicinal properties of the HDAC inhibitor according to the present invention, in particular 4SC-202, and its respective salts, as well as for the present immune checkpoint inhibitors, are described in detail in the prior art, including but not limited to the references cited herein.

In certain embodiments of the present invention, the HDAC inhibitor and the present immune checkpoint inhibitors may be administered simultaneously, sequentially or separately.

Administration of the HDAC inhibitor may in certain embodiments be within 30 minutes after a breakfast or light breakfast or within 30 minutes after a dinner or light dinner.

In the further context of the present invention, the term “active agents” refers to a pharmaceutical agent exerting a medical effect on a disease or medical condition (e.g. an amelioration thereof) and said term in particular includes the HDAC inhibitor and the present immune checkpoint inhibitors.

In the embodiments of the present invention, the active agents may be provided in pharmaceutical compositions comprising one or more of said active agents and a pharmaceutically acceptable carrier or diluent. In particular, the HDAC inhibitor and the present immune checkpoint inhibitors may be provided in separate pharmaceutical.

Such pharmaceutical compositions may be provided in the context of pharmaceutical products, comprising e.g. one or more pharmaceutical compositions and packaging material. Said packaging material typically comprises a label or package insert which indicates that the active agent(s) is/are useful for treating the diseases detailed herein. The packaging material, label and package insert otherwise parallel or resemble what is generally regarded as standard packaging material, labels and package inserts for pharmaceuticals having related utilities.

The pharmaceutical compositions according to this invention are prepared by processes which are known per se and familiar to the person skilled in the art. As pharmaceutical compositions, the active agents are either employed as such, or particularly in combination with suitable pharmaceutical auxiliaries and/or excipients, e.g. in the form of tablets, coated tablets, capsules, caplets, suppositories, patches (e.g. as TTS), emulsions, suspensions, gels or solutions, the active agent content advantageously being between 0.1 and 95% and where, by the appropriate choice of the auxiliaries and/or excipients, a pharmaceutical administration form (e.g. a delayed release form or an enteric form) exactly suited to the active agent and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other excipients, for example antioxidants, dispersants, emulsifiers, preservatives, solubilizers, colorants, complexing agents or permeation promoters, can be used.

In practicing the present invention and depending on the details, characteristics or purposes of their uses mentioned above, the active agents according to the present invention may be administered in combination therapy separately, sequentially, simultaneously or chronologically staggered (e.g. as combined unit dosage forms (in the case of the present immune checkpoint inhibitors being orally available, e.g. small molecules)), as separate unit dosage forms or adjacent discrete unit dosage forms, as fixed (in the case of the present immune checkpoint inhibitors being orally available, e.g. small molecules) or non-fixed combinations, as kit-of-parts or as admixtures (in the case of the present immune checkpoint inhibitors being orally available, e.g. small molecules)).

A “fixed combination” is defined as a combination wherein a first active ingredient and at least one further active ingredient are present together in one unit dosage or in a single entity (in the case of the present immune checkpoint inhibitors being orally available, e.g. small molecules). One example of a “fixed combination” is a pharmaceutical composition wherein the said first active ingredient and said further active ingredient are present in admixture for simultaneous administration, such as in a single formulation. Another example of a “fixed combination” is a pharmaceutical combination wherein the said first active ingredient and the said further active ingredient are present in one unit without being in admixture.

A “kit-of-parts” is defined as a combination wherein the said first active ingredient and the said further active ingredient are present in more than one unit. One example of a “kit-of-parts” is a combination wherein the said first active ingredient and the said further active ingredient are present separately. The components of the kit-of-parts may be administered separately, sequentially, simultaneously or chronologically staggered.

The first and further active ingredient of a combination or kit-of-parts according to this invention may be provided as separate formulations (i.e. independently of one another), which are subsequently brought together for simultaneous, sequential, separate or chronologically staggered use in combination therapy; or packaged and presented together as separate components of a combination pack for simultaneous, sequential, separate or chronologically staggered use in combination therapy.

The type of pharmaceutical formulation of the first and further active ingredient of a combination or kit-of-parts according to this invention can be similar, i.e. both ingredients are formulated in separate tablets or capsules, or can be different, i.e. suited for different administration forms, such as e.g. one active ingredient is formulated as tablet or capsule and the other is formulated for e.g. parenteral, in particular intravenous administration.

A further aspect of the present invention is a combination comprising, in non-fixed form, the HDAC inhibitor and one or more further therapeutic agents for sequential, separate, simultaneous or chronologically staggered use in therapy in any order. Optionally said combination comprises instructions for its use in therapy.

A further aspect of the present invention is a combined preparation, such as e.g. a kit of parts, comprising a preparation of the HDAC inhibitor and a pharmaceutically acceptable carrier or diluent and one or more further therapeutic agents; and optionally instructions for simultaneous, sequential, separate or chronologically staggered use in therapy.

A further aspect of the present invention is a kit of parts comprising a dosage unit of the HDAC inhibitor, a dosage unit of one or more further therapeutic agents, and optionally instructions for simultaneous, sequential or separate use in therapy.

A further aspect of the present invention is a pharmaceutical product comprising the HDAC inhibitor, or one or more pharmaceutical compositions comprising said compounds; and one or more further therapeutic agents, or one or more pharmaceutical compositions comprising said therapeutic agents, for simultaneous, sequential or separate use in therapy. Optionally this pharmaceutical product comprises instructions for use in said therapy.

A further aspect of the present invention is a pharmaceutical composition as unitary dosage form comprising, in admixture, the HDAC inhibitor one or more further therapeutic agents and optionally a pharmacologically acceptable carrier, diluent or excipient.

A further aspect of the present invention is a commercial package comprising the HDAC inhibitor together with instructions for simultaneous, sequential or separate use with one or more further therapeutic agents.

In addition, the combination according to the present invention can be used in the pre- or post-surgical treatment.

In further addition, the combination according to the present invention can be used in combination with radiation therapy, in particular in sensitization of patients towards standard radiation therapy.

The administration of the combination according to the present invention and pharmaceutical compositions according to the invention may be performed in any of the generally accepted modes of administration available in the art. Illustrative examples of suitable modes of administration include intravenous, oral, nasal, parenteral, topical, transdermal and rectal delivery. In a particular embodiment of the present invention, the administration of the HDAC inhibitor is via oral delivery and the administration of the present immune checkpoint inhibitors, particularly all of the present immune checkpoint inhibitors, is parenteral, in particular intravenous in the case of a biological, via oral delivery, e.g. in the case of a small molecule.

In the embodiments of the present invention, doses refer to the amount of compound with respect to the free form of said compound, i.e. the free acid or free base form of said compound. Consequently, adducts, salts, etc. of such free acid or free base form are actually to be administered in a correspondingly higher dose in order to account for the weight of the counterion or adduct partner. For example, in relation to 4SC-202 tosylate salt, a “dose of 100 mg 4SC-202” relates to (rounded) 138 mg 4SC-202 tosylate salt - comprising 100 mg 4SC-202 free base and 38 mg toluenesulfonic acid (molecular weight of 4SC-202 = 447.513 g/mol; molecular weight of 4SC-202 tosylate salt = 619.711 g/mol; therefore 100 : 447.513 * 619.711 = 138).

Having described the invention in detail, the scope of the present invention is not limited only to those described characteristics or embodiments. As will be apparent to persons skilled in the art, modifications, analogies, variations, derivations, homologisations and adaptations to the described invention can be made on the base of art-known knowledge and/or, particularly, on the base of the disclosure (e.g. the explicit, implicit or inherent disclosure) of the present invention without departing from the spirit and scope of this invention as defined by the scope of the appended claims.

As used herein, the term including, unless specified otherwise, is to be understood to mean “including, but not limited to”. Similarly, “comprising” is to be understood to generally not be limited to the items listed in each instance, but to potentially include further items.

As used herein and in the appended claims, the singular forms “a,” “an,” and “the” include plural references unless indicated otherwise. For example, “an” excipient includes one or more excipients.

The term “about” as used herein in reference to a value in particular encompasses from 90% to 110% of said value, more particularly 95% to 105% of said value.

As used herein, expressions such as “is/are administered” likewise refer to “is/are to be administered”.

“inhibition” of a response or parameter includes blocking and/or suppressing that response or parameter when compared to otherwise same conditions except for a parameter of interest, or alternatively, as compared to another condition, e.g., decrease in PD-1-signaling in the presence of a PD-1 ligand and a PD-1 inhibitor as compared to the presence of the PD-1 ligand in the absence of the PD-1 inhibitor.

An “effective amount” of an agent disclosed herein is an amount sufficient to carry out a specifically stated purpose. An “effective amount” may be determined empirically in relation to the stated purpose. An “effective amount” or an “amount sufficient” of an agent is that amount adequate to affect a desired biological effect, such as a beneficial result, including a beneficial clinical result. The term “therapeutically effective amount” refers to an amount of an agent effective to “treat” a disease or disorder in a subject (e.g., a mammal such as a human). In the case of a combination therapy, an “effective amount” refers to the amounts of the active agents of the combination (e.g., HDAC inhibitor and e.g., a LAG-3 inhibitor and a PD-1 inhibitor or PD-L1 inhibitor), which together are effective to “treat” a disease or disorder in a subject. An “effective amount” or an “amount sufficient” of an agent or agents may be administered in one or more doses.

The terms “treating” or “treatment” of a disease refer to executing a protocol, which may include administering one or more drugs to an individual (human or otherwise), in an effort to alleviate a sign or symptom of the disease. Thus, “treating” or “treatment” does not require complete alleviation of signs or symptoms, does not require a cure, and specifically includes protocols that have only a palliative effect on the individual. As used herein, and as well-understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival of an individual not receiving treatment. “Palliating” a disease or disorder means that the extent and/or undesirable clinical manifestations of the disease or disorder are lessened and/or time course of progression of the disease or disorder is slowed, as compared to the expected untreated outcome. Further, palliation and treatment do not necessarily occur by administration of one dose, but often occur upon administration of a series of doses.

For clarification, the use according the present invention of an HDAC inhibitor in combination with a LAG-3 inhibitor and a PD-1 inhibitor or PD-L1 inhibitor, and the like, are meant to relate to the use of a triple combination of an HDAC inhibitor, a LAG-3 inhibitor and a PD-1 inhibitor, or the triple combination of an HDAC inhibitor, a LAG-3 inhibitor and a PD-L1 inhibitor.

As used herein, treatment of cancer may likewise refer to treatment of a subject suffering from cancer, e.g. a human subject suffering from cancer, or a cancer patient.

In the present invention, the administration of active agents may follow a certain schedule, which may include periods of daily administration of active agents and periods wherein only one of the active agents or no active agents are administered. Particularly, such a schedule consists of treatment cycles, wherein typically such treatment cycles can be repeated as often as necessary, i.e. as seen fit by the physician responsible for the treatment.

Treatment cycles are particularly 12-16 day treatment cycles, 2 week treatment cycles, 17-25 day treatment cycles, 3 week treatment cycles, 24-32 day treatment cycles, 4 week treatment cycles, 30-40 day treatment cycles, 5 week treatment cycles, 37-47 day treatment cycles, or 6 week treatment cycles, more particularly 2 week, 3 week, 4 week, 5 week, or 6 week treatment cycles, even more particularly 2 week, 3 week, or 4 week treatment cycles, yet even more particularly 2 week or 3 week treatment cycles, yet even more particularly 3 week treatment cycles.

In certain embodiments, in the case of the present immune checkpoint inhibitors being biologicals, they are administered only on day one of each treatment cycle. The treatment cycle then follows the usual administration cycle of the present immune checkpoint inhibitors, e.g. as typically applied by physicians and/or as approved by the governmental authorities. In the case of the present immune checkpoint inhibitors being orally available, e.g. small molecules, they typically can be administered following the administration schedule of the HDAC inhibitor or continuously, or following a different pattern, e.g. a different pattern of the ones described herein for the HDAC inhibitor (e.g. the HDAC inhibitor being dosed every other day and the orally available present immune checkpoint inhibitors being administered daily.

In particular embodiments the treatment may involve a first treatment cycle, wherein only the HDAC inhibitor is administered, (i.e. none of the present immune checkpoint inhibitors are administered). Said first treatment cycle is then followed by the treatment with the combination of the present immune checkpoint inhibitors and the HDAC inhibitor as detailed herein. Said treatment cycle may otherwise (except for the administration of the present immune checkpoint inhibitors) be equal in duration and dosing of the HDAC inhibitor to the treatment cycle as described herein.

In certain embodiments, in each treatment cycle, the HDAC inhibitor may be administered for a certain number of days, followed by a number of days wherein no HDAC inhibitor is administered. In particular embodiments, the HDAC inhibitor is administered daily for 14 days in a three-week treatment cycle, or daily for 7 days in a two-week treatment cycle, in each case followed by 7 days wherein no HDAC inhibitor is administered.

In other particular embodiments, the HDAC inhibitor is administered continuously, i.e. the daily dose is administered every day during the duration of the treatment.

In particular embodiments, the PD-1 or PD-L1 inhibitor is administered on day 1 of a 2-week or 3-week treatment cycle.

In particular embodiments, the LAG-3 inhibitor is administered every 2-4 days, particularly every 3 days.

References and claims to the use of a certain compound for the manufacture of a medicament for the treatment of cancer in to be used combination with further certain agents in their general and specific forms likewise relate to:
a) the use of said compound for the manufacture of a medicament for the treatment of cancer in combination with said further certain agents;
b) methods of treating said disease or medical condition, said method comprising administering a therapeutically effective amount of said certain compound to a subject in need thereof, and administering a therapeutically effective amount of said further certain agents to said subject;
c) methods of treating said disease or medical condition, said method comprising administering a therapeutically effective amount of said certain compound to a subject in need thereof, said certain compound to be used in combination with said further certain agents;
d) compositions comprising said certain compound for the treatment of said disease or medical condition in combination with said further certain agents;
e) compositions comprising said certain compound for the treatment of said disease or medical condition, said composition to be used in combination with said further certain agents;
f) said certain compound for use in the treatment of said disease or medical condition in combination with said further certain agents;
g) said certain compound for use in the treatment of said disease or medical condition to be used in combination with said further certain agents;
and vice versa.

### EXAMPLES

The following examples serve to illustrate the invention further without restricting it.

### Materials and Methods

### 1.1. Test Substances

4SC-202 (tosylate salt).
Anti-PD1 antibody (clone: RMP1-14, catalog: BE0146, isotype: Rat IgG2a, Bioxcell),
Anti-LAG-3 antibody (clone: C9B7W, catalog: BE0174, isotype: Rat lgG1, Bioxcell),

### 1.2. Vehicles

4SC-202 was suspended in 2% aqueous methocel solution at 2 mg/ml (active compound).
Anti-PD-1 and anti-LAG3 antibody was diluted with PBS in order to reach final concentration of 1 mg/ml.

### 1.3. Treatment Doses

4SC-202 was administered at 20 mg/kg twice daily, based on 4SC-202 free base.
The anti-PD-1 and anti-LAG3 antibody was injected at 10 mg/kg per injection,

### 1.4. Routes of Administration

4SC-202 was administered by oral gavage (per os, PO) via a gavage tube.
The antibodies were injected intraperitoneally into the peritoneal cavity of mice.
In all cases, the administration volume was 10 mUkg adjusted to the most recent individual body weight of mice.

### 1.5. Cancer Cell Line and Culture Conditions

### 1.5.1. Cancer Cell Line

Colon 38 (C38, a C57BL/6J mouse colon adenocarcinoma cell line) tumor fragments.

### 1.5.2. In Vivo Tumor Amplification

The C38 fragments are stored frozen in DMSO/SVF/RPMI 1640 medium (10/10/80) in liquid nitrogen until use. In the study part 1, C38 frozen fragments were thawed at 37° C. for 5 min, rinsed twice in RPMI 1640 medium before subcutaneous (SC) implantation in mice.

### 1.6. Use of Animals

### 1.6.1. Animals

Animals were maintained in SPF health status according to the FELASA guidelines,
Animal housing and experimental procedures were realized according to the French and European Regulations and NRC Guide for the Care and Use of Laboratory Animals [2, 3],
Animals were individually identified with RFID transponder,
Each cage was labeled with a specific code.

### 1.6.2. Housing Conditions [2, 3]

Animals were maintained in housing rooms under controlled environmental conditions:
Temperature: 22 ± 2° C.,
Humidity 55 ± 10%,
Photoperiod (12 h light/12 h dark),
HEPA filtered air,
15 air exchanges per hour with no recirculation.
Animal enclosures provided sterile and adequate space with bedding material, food and water, environmental and social enrichment (group housing): Top filter polycarbonate Eurostandard Type III or IV cages; Corn cob bedding (ref: LAB COB 12, SERLAB, France); 25 kGy Irradiated diet (Ssniff®Soest, Germany); Complete food for immunodeficient rodents - NM Extrudate; Complete food for immunocompetent rodents - R/M-H Extrudate; Sterile, filtrated at 0.2 µm water; Environmental enrichment (SIZZLE-dri kraft - D20004 SERLAB, France).

### 1.6.3. Induction of C38 Tumors in Animals

Thirty-five (35) female C57BL/6J mice were subcutaneously implanted into the right flank with C38 tumor fragments. When tumor volumes reached 500-1000 mm³, tumors were surgically excised and tumor fragments (30-50 mg) were subcutaneously implanted into the right flank of 224 female C57BL/6J mice at D0.

### 1.6.4. Treatment Schedule

The treatment started when the tumors reached a mean volume of 100-200 mm³ (day 10). Animals were randomized according to their individual tumor volume into groups each of 20 animals using Vivo Manager® software (Biosystemes, Couternon, France). A statistical test (analysis of variance, ANOVA) was performed to test for homogeneity between groups. The treatment schedule was chosen as follows:
Animals from group 1 received twice daily PO administrations of vehicle for 24 consecutive days (2Q1Dx24). The bi-daily treatments were separated by a 12-hour period,
Animals from group 2 received twice daily PO administrations of 4SC-202 at 20 mg/kg per administration for 24 consecutive days (2Q1Dx24). The bi-daily treatments were separated by a 12-hour period,
Animals from group 3 received one IP injection of anti-PD1 at 10 mg/kg twice weekly for two consecutive weeks (TWx2),
Animals from group 4 received one IP injection of anti-LAG-3 at 10 mg/kg every 3 days four times (Q3Dx4),
Animals from group 5 received one IP injection of anti-PD1 at 10 mg/kg twice weekly for two consecutive weeks (TWx2) in combination with one IP injection of anti-LAG-3 at 10 mg/kg every 3 days four times (Q3Dx4). The day of concomitant injection, anti-PD1 was injected first following by injection of anti-LAG-3 within 15 minutes,
Animals from group 6 received twice daily PO administrations of 4SC-202 at 20 mg/kg per administration for 24 consecutive days (2Q1Dx24) in combination with one IP injection of anti-PD1 at 10 mg/kg twice weekly for two consecutive weeks (TWx2). The bi-daily treatments were separated by a 12-hour period. The anti-PD1 antibody treatment was performed 6 hours after the 4SC-202 morning treatment,
Animals from group 7 received twice daily PO administrations of 4SC-202 at 20 mg/kg per administration for 24 consecutive days (2Q1Dx24) in combination with one IP injection of anti-LAG-3 at 10 mg/kg every 3 days four times (Q3Dx4). The bi-daily treatments were separated by a 12-hour period. The anti-LAG-3 antibody treatment was performed 6 hours after the 4SC-202 morning treatment,
Animals from group 8 received twice daily PO administrations of 4SC-202 at 20 mg/kg per administration for 24 consecutive days (2Q1Dx24) in combination with one IP injection of anti-PD1 at 10 mg/kg twice weekly for two consecutive weeks (TWx2) and in combination with one IP injection of anti-LAG-3 at 10 mg/kg every 3 days four times (Q3Dx4). The bi-daily treatments were separated by a 12-hour period. The anti-PD1 and anti-LAG-3 antibody treatments was performed 6 hours after the 4SC-202 morning treatment. The day of concomitant injection of anti-PD1 and anti-LAG-3, anti-PD1 was injected first following by injection of anti-LAG-3 within 15 minutes.

### 1.6.5. Tumor and Clinical Monitoring

All study data, including animal body weight measurements, tumor volume, clinical and mortality records, and treatment were scheduled and recorded on Vivo Manager® database (Biosystemes, Dijon, France).

The viability and behavior were recorded every day. Body weights were measured twice a week. The length and width of the tumor were measured twice a week with calipers and the volume of the tumor were estimated by the formula:

$$\text{Tumor} \text{Volume} = {\text{width}}^{2} \times \text{length} / 2$$

### CONCLUSION

The data (data past day 27 not shown) demonstrates that whereas anti-PD-1 and 4SC-202 as monotherapies reduced tumor growth to a certain extent (groups C and E), anti-LAG3 antibody alone (group B) was not efficacious. Remarkably, the combination of 4SC-202 with anti-PD-1 (group G, double combination) demonstrated a benefit over 4SC-202 and anti-PD-1 alone (groups C and E).

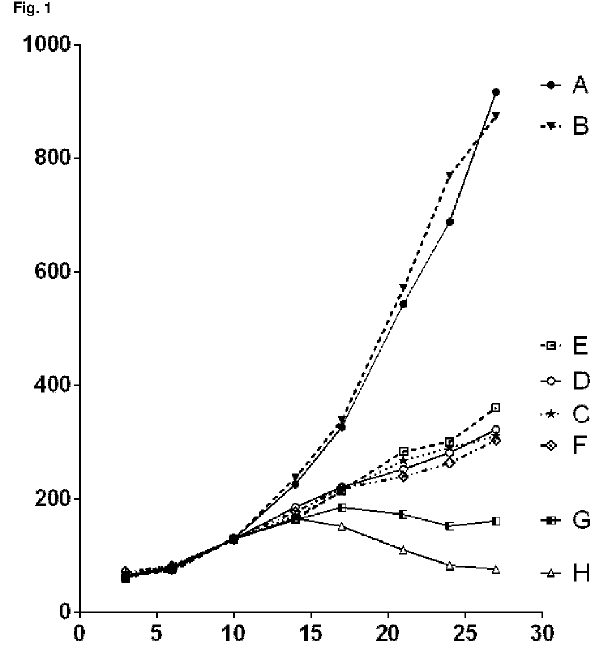

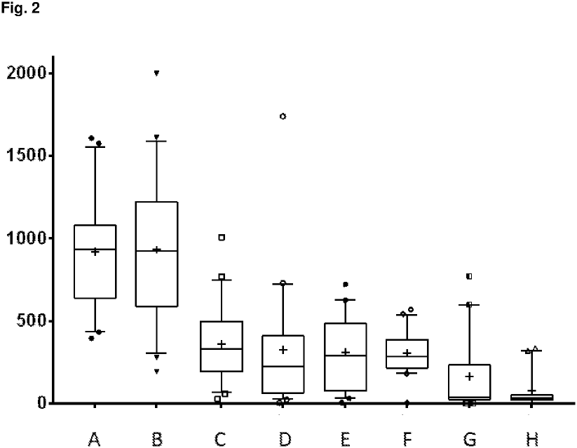

Most remarkably, while addition of anti-LAG-3 antibody to anti-PD-1 (group D) or to 4SC-202 (group F) didn’t result in significantly increased anti-tumoral efficacy over the single treatments (groups C and E), the addition anti-LAG-3 antibody to the combination of anti-PD-1 + 4SC-202 (group H, triple combination) unexpectedly resulted in a further marked increase in tumor growth reduction compared to the anti-PD-1 + 4SC-202 combination (group G, double combination), and in the overall best anti-tumoral efficacy (see FIGS. 1 and 2).

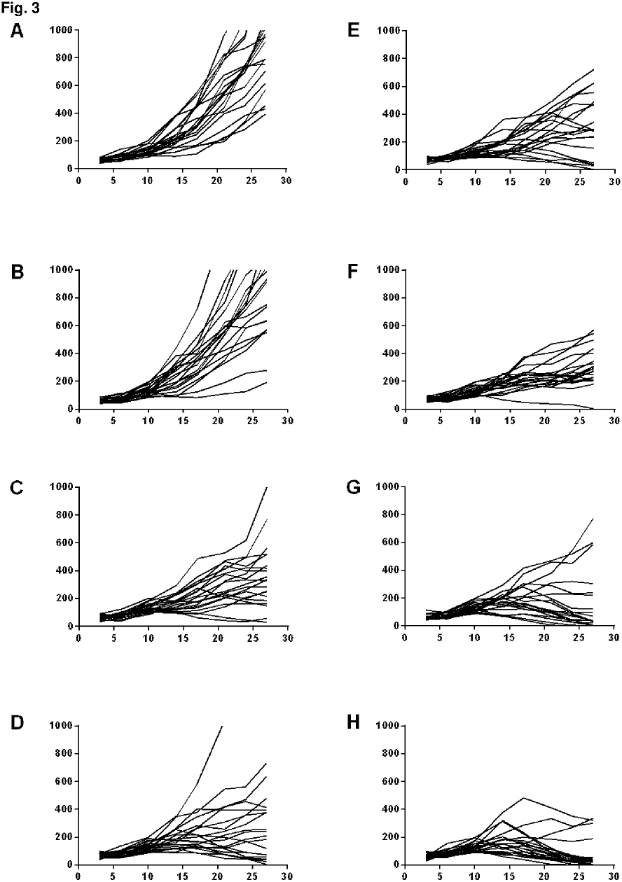

Triple combination treatment with 4SC-202, anti-PD-1, and anti-LAG3 antibodies resulted in best anti-tumoral response with nearly all tumors (18/20) markedly regressing, and a significant number of complete remissions (see FIG. 3).

1. A method for the treatment of cancer, comprising administering an effective amount of an HDAC inhibitor in combination with a LAG-3 inhibitor and a PD-1 inhibitor or PD-L1 inhibitor to a subject in need thereof.

2. The method according to claim 1, wherein the HDAC inhibitor is class I HDAC specific.

3. The method according to claim 1, wherein the HDAC inhibitor is a molecule of formula I 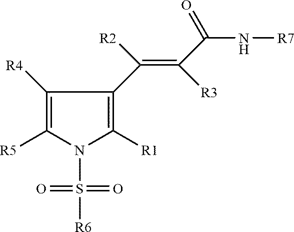
in which
R1, R4 and R5 are independently hydrogen, 1-4C-alkyl, halogen, or 1-4C-alkoxy,
R2 and R3 are independently hydrogen or 1-4C-alkyl,
R6 is -T1 -Q1, in which T1 is a bond or 1-4C-alkylene,
either Q1 is substituted by R61 and/or R62, and is Aa1, Hh1, Ha1, Ha2, Ha3, Ha4 or Ah1, or Q1 is unsubstituted, and is Ha2, Ha3 or Ha4,
in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611 )R612, -U-T3-N(R613)R614, -T4-Het3, or -V-T5-Het4, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl, 1-4C-alkylcarbonyl, or 1-4C-alkylsulphonyl
R614 is hydrogen or 1-4C-alkyl,
or R613 and R614 together and with inclusion of the nitrogen atom, to which they are bonded, form a heterocyclic ring Het2, in which
Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
T4 is a bond or 1-4C-alkylene,
Het3 is 1 N-(1 -4C-alkyl)-piperidinyl or 1 N-(1 -4C-alkyl)-pyrrolidinyl,
V is —O— (oxygen) or —C(O)NH—,
T5 is a bond or 1-4C-alkylene,
Het4 is 1 N-(1 -4C-alkyl)-piperidinyl or 1 N-(1 -4C-alkyl)-pyrrolidinyl,
R62 is 1-4C-alkyl, 1-4C-alkoxy or halogen,
Aa1 is a bisaryl radical made up of two aryl groups, which are selected independently from a group consisting of phenyl and naphthyl, and
which are linked together via a single bond,
Hh1 is a bisheteroaryl radical made up of two heteroaryl groups, which are selected independently from a group consisting of monocyclic 5- or 6-membered heteroaryl radicals comprising one or two heteroatoms, each of which is selected from the group consisting of nitrogen, oxygen and sulfur, and which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group,
R7 is hydroxyl, or Cyc1, in which Cyc1 is a ring system of formula Ia 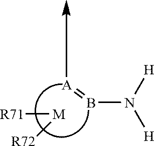
in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring, Har2 is a monocyclic 5- or 6-membered unsaturated heteroaromatic ring comprising one to three heteroatoms, each of which is selected from the group consisting of nitrogen, oxygen and sulfur,
or a molecule of the general formula III: 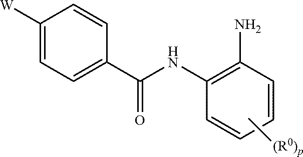
wherein:
R⁰ is halogen;
p is 0, 1, or 2;
W is —NHC(O)(C₁—C₆ alkyl) or —CH₂NH—V; and
V is —CO₂—CH₂—(heteroaryl), —C(O)—CH═CH—(heteroaryl), or heteroaryl optionally substituted by 1-3 heteroaryl groups
or a salt or solvate thereof.

4. The method according to claim 3, wherein the HDAC inhibitor is a molecule of the general formula I, or a salt or solvate thereof.

5. The method according to claim 1, wherein the HDAC inhibitor is (E)-N-(2-aminophenyl)-3-(1-((4-(1-methyl-1H-pyrazol-4-yl)phenyl)sulfonyl)-1H-pyrrol-3-yl)acrylamide or a salt or solvate thereof.

6. The method according to claim 1, wherein the HDAC inhibitor is (E)-N-(2-aminophenyl)-3-(1 -((4-(1 -methyl-1H-pyrazol-4-yl)phenyl)sulfonyl)-1 H-pyrrol-3-yl)acrylamide tosylate salt.

7. The method according to claim 1, wherein the LAG-3 inhibitor is selected from the group consisting of IMP761 IMP701, IMP731, Sym022, YBL-011, TJA3, relatlimab LAG-525 REGN-3767, Bl-754111 MK-4280, TSR-033, MGD-013, AM-0003, FS-118, XmAb-22841 and AVA-017.

8. The method according to claim 1, wherein the PD-1 inhibitor is selected from the group consisting of pidilizumabAMP-224, AB122, AMP-224, MEDI-5752, PD1-PIK, PF-06936308, RG-7769, F-520, CAB PD-1 Abs, AK-123, MEDI-3387, MEDI-5771, 4H1128Z-E27, REMD-288, SG-001, BY-24.3, CB-201, IBI-319, ONCR-177-, Max-1, CS-4100, JBI-426, CCC-0701, CCX-4503, nivolumab, pembrolizumab, BCD-100, cemiplimab, sintilimab, JNJ-3283, spartalizumab, camrelizumab, tisielizumab, AGEN-2034, MEDI-0680, toripalimab, dostarlimab, ABBV-181, AK-104,AK-105, BAT-1306, BI-754091, CBT-501, Genolimzumab, GLS-010 , LZM-009, MGA-012, MGD-013, PF-06801591, Sym-021, CS-1003, HLX-10, AK-103, AM-0001, TILT-123, BH-2922, BH-2941, BH-2950, CX-188, ENUM-244C8 , ENUM-388D4, HAB-21, HEISCOIII-003, IKT-202, JS-003 , JTX-4014 ,MCLA-134, MGD-019, MT-17000, PEGMP-7, PRS-332,RXI-762, STI-1110, VXM-10, XmAb-20717, XmAb-23104, AK-112, HLX-20, SSI-361, AT-16201, and SNA-01.

9. The method according to claim 1, wherein the PD-L1 inhibitor is selected from the group consisting of BCD-135, APL-502, MDX-1105 ,IMC-001, KD-045, INBRX-105, KN-046, INCB-086550, IMC-2102, IMC-2101, anti-PDL1-TGFbRIIecd, KD-005, PM-PDL-GEX, IMM-2502, 89Zr-CX-072, KY-1055, MEDI-1109, MT-5594, 89Zr-DFO-6E11, SL-279252, DSP-106, Gensci-047, REMD-290, N-809, PRS-344, FS-222, GEN-1046, BH-29xx, atezolizumab, avelumab, durvalumab, BGB-A333, CX-072, GNS-1480, AMP-224, CA-170, CK-301, CS-1001, FAZ-053, envafolimab, LY-3300054, M-7824, HTI-1088, MSB-2311, STI-A1014, AK-106, AVA-004, BBI-801, CA-327, CBA-0710, CBT-502, FPT-155, FS-118, IKT-201, IKT-703, IO-103, JS-003, KD-033 KY-1003, MCLA-145, MT-5050 and SNA-02.

10. The method according to claim 1, wherein said cancer is selected from the group consisting of melanoma (in particular ocular and uveal, but also including skin melanoma), head and neck, renal, Non-small cell lung cancer (NSCLC), microsatellite-instable carcinoma (lynch syndrome, in particular gastroesophageal and colorectal), urothelial carcinoma including bladder cancer, merkel cell carcinoma, hodgkin lymphoma, gastric, gastrointestinal cancers (microsatellite stable and instable) including colorectal cancer (CRC), hepatocellular carcinoma (HCC), renal cell carcinoma (RCC), nasopharyngeal, basal cell carcinoma, cervical cancer, anogenital cancers, Kaposi sarcoma, adult T-cell leukemia, primary effusion lymphoma, and Castlemann’s disease, or selected from the group consisting of breast cancer, in particular triple-negative breast cancer, oesophageal cancer, non-hodgkin lymphoma, small cell lung cancer (SCLC), sarkoma, mesothelioma, glioblastoma, microsatellite stable cancer (in particular gastroesophageal and colorectal), pancreas cancer, prostate cancer, cutaneous T-cell lymphoma (CTCL), and squamous cell carcinoma.

11. (canceled)

12. The method according to claim 1,
wherein said HDAC inhibitor is class I HDAC specific,
said LAG-3 inhibitor is selected from the group consisting of IMP761, IMP701, IMP731, Sym022, YBL-011, TJA3, relatlimab, LAG-525. REGN-3767. BI-754111, MK-4280, TSR-033, MGD-013, AM-0003, FS-118, XmAb-22841 and AVA-017,
said PD-1 inhibitor is selected from the group consisting of pidilizumab, AMP-224, AB122, AMP-224, MEDI-5752, PD1-PIK, PF-06936308, RG-7769 , F-520, CAB PD-1 Abs , AK-123 , MEDI-3387, MEDI-5771, 4H1128Z-E27 , REMD-288, SG-001 , BY-24.3, CB-201, IBI-319, ONGR-177, Max-1, CS-4100, JBI-426, CCC-0701, CCX-4503, nivolumab, pembrolizumab, BCD-100, cemiplimab, sintilimab, JNJ-3283, spartalizumab, camrelizumab, tislelizumab, AGEN-2034, MEDI-0680, toripalimab, dostarlimab, ABBV-181, AK-104. AK-105, BAT-1306, BI-754091, CBT-501. Genolimzumab, GLS-010, LZM-009. MGA-012, MGD-013, PF-06801591, Sym-021, CS-1003, HLX-10, AK-103, AM-0001, TILT-123, BH-2922, BH-2941, BH-2950. CX-188, ENUM-244C8, ENUM-388D4, HAB-21, HEISCOIII-003, IKT-202, JS-003, JTX-4014, MCLA-134, MGD-019, MT-17000, PEGMP-7, PRS-332, RXI-762. STI-1110, VXM-10, XmAb-20717, XmAb-23104, AK-112, HLX-20, SSI-361, AT-16201 and SNA-01, and
said PD-L1 inhibitor is selected from the group consisting of BCD-135, APL-502, MDX-1105, IMC-001, KD-045, INBRX-105, KN-046, INCB-086550. IMC-2102, IMC-2101, anti-PDL1-TGFbRllecd, KD-005, PM-PDL-GEX, IMM-2502, 89Zr-CX-072, KY-1055, MEDI-1109, MT-5594, 89Zr-DFO-6E11, SL-279252 , DSP-106, Gensci-047, REMD-290, N-809, PRS-344, FS-222, GEN-1046, BH-29xx, atezolizumab, avelumab, durvalumab, BGB-A333, CX-072 , GNS-1480 , AMP-224, CA-170, CK-301, CS-1001, FAZ-053, envafolimab, LY-3300054, M-7824, HTI-1088 , MSB-2311, STI-A1014, AK-106, AVA-004, BBI-801, CA-327, CBA-0710 , CBT-502, FPT-155, FS-118, IKT-201, IKT-703, IO-103, JS-003, KD-033, KY-1003, MCLA-145, MT-5050 and SNA-02.

13-14. (canceled)

15. The method according to claim 12, wherein the HDAC inhibitor is a molecule of formula I 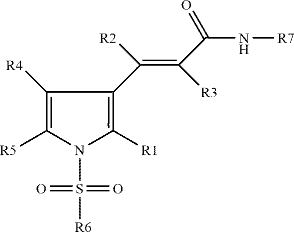
in which
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond or 1-4C-alkylene,
either Q1 is substituted by R61 and/or R62, and is Aa1, Hh1, Ha1, Ha2, Ha3, Ha4 or Ah1, or Q1 is unsubstituted, and is Ha2, Ha3 or Ha4,
in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, -U-T3-N(R613)R614, -T4-Het3, or -V-T5-Het4, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl, 1-4C-alkylcarbonyl, or 1-4C-alkylsulphonyl
R614 is hydrogen or 1-4C-alkyl,
or R613 and R614 together and with inclusion of the nitrogen atom, to which they are bonded, form a heterocyclic ring Het2, in which
Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
T4 is a bond or 1-4C-alkylene,
Het3 is 1N-(1-4C-alkyl)-piperidinyl or 1N-(1-4C-alkyl)-pyrrolidinyl,
V is —O— (oxygen) or —C(O)NH—,
T5 is a bond or 1-4C-alkylene,
Het4 is 1N-(1-4C-alkyl)-piperidinyl or 1N-(1-4C-alkyl)-pyrrolidinyl,
R62 is 1-4C-alkyl, 1-4C-alkoxy or halogen,
Aa1 is a bisaryl radical made up of two aryl groups, which are selected independently from a group consisting of phenyl and naphthyl, and
which are linked together via a single bond,
Hh1 is a bisheteroaryl radical made up of two heteroaryl groups, which are selected independently from a group consisting of monocyclic 5- or 6-membered heteroaryl radicals comprising one or two heteroatoms, each of which is selected from the group consisting of nitrogen, oxygen and sulfur, and which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group,
R7 is hydroxyl, or Cyc1, in which Cyc1 is a ring system of formula Ia 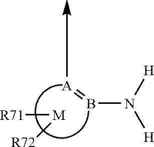
in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring, Har2 is a monocyclic 5- or 6-membered unsaturated heteroaromatic ring comprising one to three heteroatoms, each of which is selected from the group consisting of nitrogen, oxygen and sulfur,
or a molecule of the general formula III: 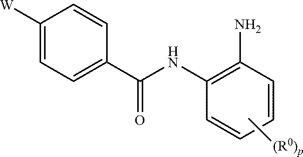
wherein:
R⁰ is halogen;
p is 0, 1, or 2;
W is —NHC(O)(C₁—C₆ alkyl) or —CH₂NH—V; and
V is —CO₂—CH₂—(heteroaryl), —C(O)—CH═CH—(heteroaryl), or heteroaryl optionally substituted by 1-3 heteroaryl groups
or a salt or solvate thereof.

16. The method according to claim 15, wherein the HDAC inhibitor is a molecule of the general formula I, or a salt or solvate thereof.

17. The method according to claim 12, wherein the HDAC inhibitor is (E)-N-(2-aminophenyl)-3-(1-((4-(1-methyl-1H-pyrazol-4-yl)phenyl)sulfonyl)-1H-pyrrol-3-yl)acrylamide or a salt or solvate thereof.

18. The method according to claim 12, wherein the HDAC inhibitor is (E)-N-(2-aminophenyl)-3-(1 -((4-(1-methyl-1H-pyrazol-4-yl)phenyl)sulfonyl)-1H-pyrrol-3-yl)acrylamide tosylate salt.

19. The method according to claim 12, wherein said cancer is selected from the group consisting of melanoma (in particular ocular and uveal, but also including skin melanoma), head and neck, renal, Non-small cell lung cancer (NSCLC), microsatellite-instable carcinoma (lynch syndrome, in particular gastroesophageal and colorectal), urothelial carcinoma including bladder cancer, merkel cell carcinoma, hodgkin lymphoma, gastric, gastrointestinal cancers (microsatellite stable and instable) including colorectal cancer (CRC), hepatocellular carcinoma (HCC), renal cell carcinoma (RCC), nasopharyngeal, basal cell carcinoma, cervical cancer, anogenital cancers, Kaposi sarcoma, adult T-cell leukemia, primary effusion lymphoma, and Castlemann’s disease, or selected from the group consisting of breast cancer, in particular triple-negative breast cancer, oesophageal cancer, non-hodgkin lymphoma, small cell lung cancer (SCLC), sarkoma, mesothelioma, glioblastoma, microsatellite stable cancer (in particular gastroesophageal and colorectal), pancreas cancer, prostate cancer, cutaneous T-cell lymphoma (CTCL), and squamous cell carcinoma.
